(19) United States
(12) Patent Application Publication
(10) Pub. No.: US 2009/0075937 A1
(43) Pub. Date: Mar. 19, 2009

(54) LHRH ANTAGONISTS FOR THE TREATMENT OF LOWER URINARY TRACT SYMPTOMS

(75) Inventors: Juergen ENGEL, Alzenau (DE); Oliver Bauer, Frankfurt/Main (DE)

Correspondence Address: OBLON, SPIVAK, MCCLELLAND MAIER & NEUSTADT, P.C., 1940 DUKE STREET, ALEXANDRIA, VA, 22314, US

(73) Assignee: AETERNA ZENTARIS GmbH, Frankfurt (DE)

(21) Appl. No.: 12/042,522

(22) Filed: Mar. 5, 2008

Related U.S. Application Data
(60) Provisional application No. 60/892,899, filed on Mar. 5, 2007.

(30) Foreign Application Priority Data
Mar. 5, 2007 (EP) 07103483.9

(51) Int. Cl. A61K 31/403; A61K 31/4439; A61K 31/4245; A61K 31/675; A61K 31/497; A61K 31/4725; A61P 13/00; A61K 31/4178; A61K 31/506; A61K 31/454; A61K 31/5377

(52) U.S. Cl. 514/80; 514/339; 514/364; 514/232.8; 514/411; 514/323; 514/343; 514/256; 514/255.05; 514/397; 514/310

(57) ABSTRACT

The present invention provides at least one LHRH antagonist for the treatment or prophylaxis of at least one lower urinary tract symptom in mammals which is to be administered in an intermediate dose, which does not cause chemical (hormonal) castration.

### CROSS-REFERENCE TO RELATED APPLICATIONS

This application claims the benefit of U.S. provisional application 60/892,899 filed Mar. 5, 2007 and EP 07103483.9 filed Mar. 5, 2007, both of which are incorporated herein by reference.

### DESCRIPTION

1. Field of the Invention

The invention relates to the use of LHRH antagonists for the treatment or prophylaxis of lower urinary tract symptom in mammals. Such lower urinary tract symptom include, e.g., urinary incontinence, urge incontinence, overactive bladder, idiopathic overactive bladder, neurogenic overactive bladder, detrusor overactivity, idiopathic detrusor overactivity, neurogenic detrusor overactivity and benign prostatic hyperplasia (BPH). The invention further relates to the treatment or prophylaxis of above symptoms by administering LHRH antagonists in intermediate doses, which do not cause chemical (hormonal) castration.

2. Background

Genitourinary problems, including neurogenic dysfunction, such as incontinence and urinary retention, impotence, prostatism, urinary tract infections (UTI), benign prostatic hyperplasia/hypertrophy (BPH) and prostate cancer, are common in the elderly, and most of the symptoms can be alleviated through pharmacological management (Atala A et al., Drugs & Aging 1991, 1(3): 176-193).

Overactive bladder (OAB) is a syndrome characterized by urgency with or without urge incontinence, usually with increased frequency and nocturia. Bladder overactivity can be myogenic or neurogenic in its origin or it can be idiopathic in nature. Myogenic etiology is characterized by impairment in smooth muscle function as a result of partial urethral obstruction. The increased smooth muscle signalling due to loss normal excitatory neural input leads to a state of unstable contractions or overactivity. OAB is also triggered by neurological effects resulting in dysregulation of reflexes to the bladder and urethra and leading to neurogenic detrusor overactivity.

In contrast, (urge) urinary incontinence is defined as an involuntary leakage accompanied by or immediately preceded by urgency (Tiwari A et al., Expert Opin. Investig. Drugs 2006, 15(9): 1017-1037).

In their review, Atala et al. (Atala A et al., Drugs & Aging 1991, 1(3): 176-193) mention the use of LHRH agonists, such as goserelin, leuprorelin, nafarelin and buserelin, to achieve castrate levels of androgens, for instance testosterone, in advanced prostatic carcinoma and BPH. The indication overactive bladder is not mentioned, neither are LHRH antagonists.

WO 02/36144 discloses the use of GnRH analogues for the treatment of side effects of ovariectomy or symptoms associated with reproductive senescence in female mammals, such as post-menopausal women and spayed bitches, in particular urinary incontinence, mood changes, hot flushes and skin/hair changes. The patent application mentions GnRH agonists and antagonists, but is silent about the prevention of chemical (hormonal) castration and corresponding appropriate dosage schemes. Further, WO 02/36144 is only directed to female mammals and does not disclose the treatment of male human nor does it mention overactive bladder as medicinal indication. Although WO 02/36144 mentions the theoretical use of GnRH antagonists, all examples and experimental embodiments refer to GnRH agonists used in discrete doses only: leuprolide acetate, deslorelin, triptorelin acetate, buserelin acetate in dose ranges from 3.75 mg to 12 mg. However, it is well-known to the person skilled in the art that the use of GnRH agonists in these doses inevitably leads to chemical (hormonal) castration and undesired unfavourable hormone withdrawal symptoms. Further, no experimental data are presented: the described experimental use of the GnRH agonist analogues is a mere hypothesis.

Reichler et al. describe the effect of GnRH analogues on urinary incontinence after ablation of the ovaries in dogs. Depot formulations of GnRH agonist analogues leuprolide, deslorelin, buserelin and triptorelin were used with dose ranges from 3.75 mg to 11.25 mg. Castration, i.e. reduction of gonadotropins FSH and LH to basal or undetectable levels, is reported. The authors are silent about the use of LHRH antagonists, the prevention of chemical (hormonal) castration, corresponding appropriate dosage schemes and overactive bladder as medicinal indication (Reichler I M et al., Theriogenology 2003, 60: 1207-1216).

Nitti reviews the status quo on botulinum toxin for the treatment of idiopathic and neurogenic overactive bladder and detrusor overactivity. The use of LHRH antagonists is not mentioned (Nitti V W, Reviews in Urology 2006, 8(4): 198-208).

Reichler and co-workers studied the effect of a long acting GnRH analogue or placebo on plasma LH/FSH, urethral pressure profiles and clinical signs of urinary incontinence due to sphincter mechanism incompetence in bitches. Use of GnRH agonist analogue Leuprolide acetate in 20 or 30 mg doses is reported. It is further reported that there is a wide range in the response to the GnRH agonist analogue therapy, from ineffective to long lasting, which may be due to different responses between various GnRH analogues. It is even postulated that gonadotropins FSH and LH are unlikely involved in the pathophysiology of urinary incontinence in bitches since FSH and LH levels are no different in both successfully and unsuccessfully treated dogs. The authors are silent about the use of LHRH antagonists, the prevention of chemical (hormonal) castration, corresponding appropriate dosage schemes and overactive bladder as medicinal indication (Reichler I M et al., Theriogenology 2006, 66: 1227-1236).

Reichler and co-workers examined urodynamic parameters and plasma LH/FSH in spayed Beagle bitches before and 8 weeks after GnRH agonist analogue depot treatment. GnRH analogue leuprolide in a 30 mg 4-months depot formulation was applied. The bitches have already been castrated before the treatment, gonadotropins FSH and LH were shown to be decreased to undetectable levels. The authors are silent about the use of LHRH antagonists, the prevention of chemical (hormonal) castration, corresponding appropriate dosage schemes and overactive bladder as medicinal indication (Reichler I M et al., Theriogenology 2006, 66: 2127-2136).

Kaplan et al. demonstrate efficacy of tolterodine and tamsulosin for treatment of men with lower urinary tract symptoms and overactive bladder in a randomized controlled trial (Kaplan S A et al., JAMA 2006, 296(19): 2319-2328). The use of LHRH antagonists is not mentioned.

WO 2006/129162 is directed to anti-LHRH vaccines for the control or treatment of urinary incontinence. The use of LHRH antagonists is not mentioned.

### SUMMARY OF THE INVENTION

The present invention has the object to provide novel treatments for lower urinary tract symptoms, in particular overactive bladder and/or detrusor overactivity, by which negative hormone withdrawal symptoms are minimized and/or prevented.

The object of the invention has surprisingly been solved in one aspect by providing at least one LHRH antagonist that can be used for the preparation of a medicament for the treatment or prophylaxis of at least one lower urinary tract symptom in mammals, wherein the at least one lower urinary tract symptom is urinary incontinence, urge incontinence, overactive bladder, idiopathic overactive bladder, neurogenic overactive bladder, detrusor overactivity, idiopathic detrusor overactivity, and/or neurogenic detrusor overactivity and wherein the at least one LHRH antagonist is to be administered in an intermediate dose, which does not cause chemical (hormonal) castration.

### DETAILED DESCRIPTION OF THE INVENTION

The term “overactive bladder” is defined by the International Continence Society (ICS) as follows: Overactive bladder (OAB) is a symptom complex consisting of urgency with or without urge incontinence, usually with frequency and nocturia, in the absence of local pathologic or hormonal factors (Abrams P et al., Urology 2003, 61 (1): 37-49; Abrams P et al., Urology 2003, 62 (Supplement 5B): 28-37 and 40-42). Synonyms of overactive bladder (OAB) include “urge syndrome” and “urge frequency syndrome”.

The term “detrusor overactivity” is defined by the International Continence Society (ICS) as follows: Detrusor overactivity is a urodynamic observation characterized by involuntary detrusor contractions during the filling phase that may be spontaneous or provoked (Abrams P et al., Urology 2003, 62 (Supplement 5B): 28-37 and 40-42).

An overview about the lower urinary tract symptoms “overactive bladder” and “detrusor overactivity” including their definitions, different subforms and/or different etiologies, in particular in men and women, is given in Abrams P et al. (Abrams P et al., Urology 2003, 62 (Supplement 5B): 28-37 and 40-42). From the document it is evident that patients suffering from overactive bladder almost all have coexisting daytime and nighttime frequency and many of them also urgency, whereas only up to one half of the patients have co-existing urge incontinence (FIG. 1). In woman there is also a form of “dry” overactive bladder”, where patients show urgency, increased frequency and nocturia, but not urge incontinence (FIG. 2). From this overview it is clear that “overactive bladder” and/or “detrusor overactivity” (FIG. 3) are not equal to urge incontinence, as many of the patients suffering from the former two syndromes are not incontinent, i.e. they are “dry” (see also: Tiwari A et al., Expert Opin. Investig. Drugs 2006, 15(9): 1017-1037). This document (Abrams P et al., Urology 2003, 62 (Supplement 5B): 28-37 and 40-42) is herewith explicitly incorporated by reference in its entirety and explicitly made reference to with regard to the definitions, different subforms and/or etiologies given, in particular with respect to men and women, separately.

It is therefore preferred in the course of the present invention that all these different subforms and etiologies of “overactive bladder” and/or “detrusor overactivity” are comprised. In particular it is preferred that patients suffering from “overactive bladder” and/or “detrusor overactivity” are comprised that are not incontinent, i.e. who do not show symptoms of urge incontinence, incontinence and the like, but are “dry”.

The term “intermediate dose” in the course of the present invention is defined by its higher and lower limit and has the following meaning: The higher limit of “intermediate dose” is the dose that just does not cause chemical (hormonal) castration as defined herein, wherein the lower limit of “intermediate dose” is the dose that just causes a lowering, even if a very small one, of LH, FSH and/or testosterone with regard to normal sex hormone blood levels. It lies within the knowledge of the skilled artisan to elaborate the lower and upper limit of an “intermediate dose” for each LHRH antagonist to be used on the basis of his expert knowledge and the disclosure of the present invention.

In a preferred embodiment, at least one LHRH antagonist is provided that can be used for the preparation of a medicament for the treatment or prophylaxis of at least one lower urinary tract symptom in mammals, wherein the at least one lower urinary tract symptom is not associated with benign prostatic hyperplasia (BPH).

In a preferred embodiment, at least one LHRH antagonist is provided that can be used for the preparation of a medicament for the treatment or prophylaxis of at least one lower urinary tract symptom in mammals, wherein the at least one lower urinary tract symptom is overactive bladder, idiopathic overactive bladder, neurogenic overactive bladder, detrusor overactivity, idiopathic detrusor overactivity, and/or neurogenic detrusor overactivity and wherein the at least one LHRH antagonist is to be administered in an intermediate dose, which does not cause chemical (hormonal) castration.

In another preferred embodiment, at least one LHRH antagonist is provided that can be used for the preparation of a medicament for the simultaneous treatment or prophylaxis of at least one lower urinary tract symptom overactive bladder, idiopathic overactive bladder, neurogenic overactive bladder, detrusor overactivity, idiopathic detrusor overactivity, and/or neurogenic detrusor overactivity and benign prostatic hyperplasia (BPH), wherein the at least one LHRH antagonist is to be administered in an intermediate dose, which does not cause chemical (hormonal) castration.

That is, for instance, by means of this preferred embodiment patients can be treated that suffer from overactive bladder and/or its subforms/etiologies idiopathic overactive bladder and/or neurogenic overactive bladder and benign prostatic hyperplasia (BPH).

In a further preferred embodiment, at least one LHRH antagonist is provided that can be used for the preparation of a medicament for the herein disclosed treatments, wherein the LHRH antagonist is abarelix (Chemical Abstract Services Registry Number: 183552-38-7), antide (Chemical Abstract Services Registry Number: 112568-12-4), azaline B (Chemical Abstract Services Registry Number: 134457-28-6), A-75998 (Chemical Abstract Services Registry Number: 135215-95-1), cetrorelix (Chemical Abstract Services Registry Number: 120287-85-6), degarelix (Chemical Abstract Services Registry Number: 214766-78-6), detirelix (Chemical Abstract Services Registry Number: 89662-30-6), ozarelix (D-63153) (Chemical Abstract Services Registry Number: 295350-45-7), ganirelix (Chemical Abstract Services Registry Number: 124904-93-4), NaI-Glu antagonist, ramorelix (Chemical Abstract Services Registry Number: 127932-90-5), RS-68439 (Chemical Abstract Services Registry Number: 102583-46-0), and/or teverelix (Chemical Abstract Services Registry Number: 144743-92-0) and preferably is cetrorelix or ozarelix (D-63153).

In a further preferred embodiment, at least one LHRH antagonist is provided that can be used for the preparation of a medicament for the herein disclosed treatments, wherein the LHRH antagonist is a tetrahydrocarbazole compound of the formula (I)

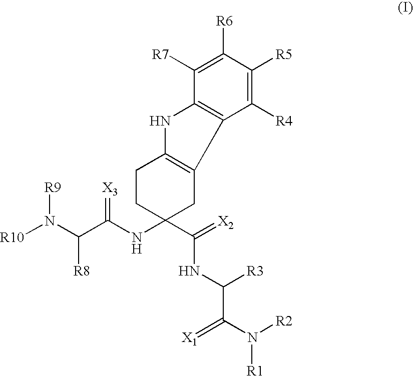

in which:
X₁ is S, O or S⁺—O⁻,
X₂ and X₃ are independently of one another O or germinally linked H₂,
R1 and R2 are independently of one another selected from the group consisting of —H, aryl, alkyl and arylalkyl radicals which are optionally substituted in the alkyl and/or aryl group by up to 3 substituents independently selected from the group consisting of —Hal, —CN and —O-alkyl, where R1 and R2 are in particular hydrogen,
R3 is an alkyl, arylalkyl or heteroarylalkyl radical, which are optionally substituted by up to 3 substituents independently selected from the group consisting of —Hal, —CN, —CO—O—R12, —CO—NR12R12′, —OH, —O—R13, —O—CO—R13, —O—SO₂—OR12, —O—SO₂—R12, —SO₂—OR12, —SO—R12, —O—PO(OR12)(OR12′), —O—PO(NR12R12′)₂, —O—CO—O—R13, —O—CO—NR12R12′, —O—CS—NR12R12′, —S—R12, —NR12R12′, —NH—CO—R13, —NH—SO₂—R12, —NH—CO—O—R13, —NH—CO—NHR12, —NH—C(NH)—NH₂,
R4, R5, R6 and R7 are selected independently of one another from the group consisting of H, —Hal, —CN, —CONH₂, —COOH, —CF₃, —O-alkyl, —OCF₃, —NO₂, and alkyl, arylalkyl and heteroarylalkyl radicals;
R9 is a hydrogen atom, an alkyl, an aryl, a heteroaryl, an arylalkyl or a heteroarylalkyl radical, preferably a hydrogen atom;
R10 is a hydrogen atom, or the radical —R11, —CO—R11, —CO—OR11, —CO—NHR11, —C(NH)—NHR11, —SO₂—R11, or —SO₂—NHR11;
R11 is an alkyl, an aryl, a heteroaryl, an arylalkyl or a heteroarylalkyl radical, which are optionally substituted by one or more substituents independently selected from the group consisting of —Hal, —CN, -alkyl, —CF₃, —OCF₃, —OH, —O-alkyl, and —O—(CH₂CH₂—O)ₙ—CH₃;
R8 is —C₁-C₆-alkyl-aryl or —C₁-C₆-alkyl-heteroaryl, where the aryl or heteroaryl group is substituted by one to three, preferably by one, substituents independently selected from the group consisting of —O—(CH₂CH₂—O)ₙ—CH₃, —O—CO—R12, —O—CO—(CH₂CH₂—O)ₙ—CH₃, —O—SO₂—OR12, —O—SO₂—R12, —O—PO(OR12)(OR12′), —O—PO(NR12R12′)₂, —O—CO—OR13, —O—CO—NR12R12′, and —O—CS—NR12R12′, or, where, however, at least
(i) X₁ is S, or
(ii) R10 is not H, and R11 is an arylalkyl or heteroarylalkyl radical, which are substituted in the aryl or heteroaryl group by one or more substituents independently selected from the group consisting of Hal, —CN, -alkyl, —CF₃, —OCF₃, —OH, —O-alkyl, and —O—(CH₂CH₂—O)ₙ—CH₃,
R8 also assumes the meanings indicated for R3;
R12 and R12′ are independently of one another H, or an alkyl, arylalkyl, aryl, heteroarylalkyl, or heteroaryl radical and are preferably H,
R13 is selected from an alkyl, arylalkyl, aryl, heteroarylalkyl, and heteroaryl radical, or is the group —(CH₂CH₂—O)ₙ—CH₃, and
n is an integer from 1 to 10, preferably 1 to 6.

In a further preferred embodiment, at least one LHRH antagonist is provided that can be used for the preparation of a medicament for the herein disclosed treatments, wherein the LHRH antagonist is selected from the group consisting of:
4-chlorobenzyl {(S)-1-[(R)-3-((S)-1-carbamoyl-2-methylpropylcarbamoyl)-6,8-dichloro-2,3,4,9-tetrahydro-1H-carbazol-3-ylcarbamoyl]-2-methylpropyl}carbamate (1),
4-chlorobenzyl {(S)-1-[(R)-3-((S)-1-carbamoyl-2-methylpropylcarbamoyl)-6,8-dichloro-2,3,4,9-tetrahydro-1H-carbazol-3-ylcarbamoyl]-2-methylbutyl}carbamate (2),
4-chlorobenzyl {(S)-1-[(R)-3-((S)-carbamoyl-2-methylbutylcarbamoyl)-6,8-dichloro-2,3,4,9-tetrahydro-1H-carbazol-3-ylcarbamoyl]-2-methylbutyl}carbamate (3),
(R)-6,8-dichloro-3-{(S)-2-[2-(2-fluorophenyl)acetylamino]-3-methyl-pentanoylamino}-2,3,4,9-tetrahydro-1H-carbazole-3-carboxylic acid ((S)-1-carbamoyl-2-methylbutyl)amide (4),
(R)-6,8-dichloro-3-{(S)-2-[2-(3-fluorophenyl)acetylamino]-3-methyl-pentanoylamino}-2,3,4,9-tetrahydro-1H-carbazole-3-carboxylic acid ((S)-1-carbamoyl-2-methylbutyl)amide (5),
2-chlorobenzyl {(S)-1-[(R)-3-((S)-carbamoyl-2-methylbutylcarbamoyl)-6,8-dichloro-2,3,4,9-tetrahydro-1H-carbazol-3-ylcarbamoyl]-2-methylbutyl}carbamate (6),
benzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-2-methylbutyl}carbamate (7),
benzyl 4-{(S)-3-benzyloxycarbonylamino-3-[(R)-3-((S)-1-carbamoyl-2-methylbutylcarbamoyl)-6,8-dichloro-2,3,4,9-tetrahydro-1H-carbazol-3-ylcarbamoyl]propyl}-phenylcarbonate (8),
benzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-2-(4-phosphonooxyphenyl)ethyl]-carbamate (9),
benzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]carbamate (10),
benzyl [(S)-1-[(R)-3-((S)-carbamoyl-2-methylbutylcarbamoyl)-6,8-dichloro-2,3,4,9-tetrahydro-1H-carbazol-3-ylcarbamoyl]-3-(4-phosphonooxyphenyl)propyl]-carbamate (11),
benzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-phosphonooxyphenyl)propyl]carbamate (12),
(R)-6,8-dichloro-3-{(S)-2-[2-(2-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (13),
(R)-6,8-dichloro-3-[(S)-2-[2-(2-fluorophenyl)acetylamino]-4-(4-hydroxyphenyl)butyryl-amino]-2,3,4,9-tetrahydro-1H-carbazole-3-carboxylic acid ((S)-2-methyl-1-thiocarbamoyl-butyl)amide (14),
mono(4-{(S)-3-[(R)-6,8-dichloro-3-((S)-2-methyl-1-thiocarbamoylbutylcarbamoyl)-2,3,4,9-tetrahydro-1H-carbazol-3-ylcarbamoyl]-3-[2-(2-fluorophenyl)acetylamino]-propyl}phenyl phosphate (15),
(R)-6,8-dichloro-3-{(S)-2-[3-(4-fluorophenyl)propionylamino]-3-methyl-pentanoylamino}-2,3,4,9-tetrahydro-1H-carbazole-3-carboxylic acid ((S)-1-carbamoyl-2-methylpropyl)amide (16),
(S)-5-[(R)-3-((S)-1-carbamoyl-2-methylpropylcarbamoyl)-6,8-dichloro-2,3,4,9-tetrahydro-1H-carbazol-3-ylcarbamoyl]-5-[3-(4-fluorophenyl)propionylamino]pentylammonium trifluoroacetate (17),
(S)-6,8-dichloro-3-{(S)-2-[3-(2-hydroxyphenyl)propionylamino]-3-methyl-pentanoylamino}-2,3,4,9-tetrahydro-1H-carbazole-3-carboxylic acid ((S)-1-carbamoyl-2-methylbutyl)amide (18),
benzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-[4-(2-{2-[2-(2-methoxyethoxy)ethoxy]-ethoxy}ethoxy)phenyl]ethyl}carbamate (19),
(R)-6,8-dichloro-3-((S)-2-{3-[2-(2-{2-[2-(2-methoxyethoxy)ethoxy]ethoxy}ethoxy)phenyl]propionylamino}-3-methyl-pentanoylamino)-2,3,4,9-tetrahydro-1H-carbazole-3-carboxylic acid ((S)-1-carbamoyl-2-methylbutyl)amide (20),
(R)-6,8-dichloro-3-((S)-2-{2-[2-(2-{2-[2-(2-methoxyethoxy)ethoxy]ethoxy}ethoxy)phenyl]acetylamino}-3-methyl-pentanoylamino)-2,3,4,9-tetrahydro-1H-carbazole-3-carboxylic acid ((S)-1-carbamoyl-2-methylbutyl)amide (21),
(R)-6,8-dichloro-3-[(S)-2-[3-(2-fluorophenyl)propionylamino]-4-(4-hydroxyphenyl)butyryl-amino]-2,3,4,9-tetrahydro-1H-carbazole-3-carboxylic acid ((S)-1-carbamoyl-2-methylbutyl)amide (22),
(R)-6,8-dichloro-3-{(S)-2-[3-(2-fluorophenyl)propionylamino]-3-methyl-pentanoylamino}-2,3,4,9-tetrahydro-1H-carbazole-3-carboxylic acid ((S)-1-carbamoyl-2-methylbutyl)amide (23),
benzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-[4-(2-{2-[2-(2-methoxyethoxy)ethoxy]-ethoxy}ethoxy)phenyl]propyl}carbamate (24),
benzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}carbamate (25),
3-methylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (26),
2,6-difluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (27),
3,5-difluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (28),
3,5-dichlorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (29),
3-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (30),
2-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (31),
3-chlorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (32),
3,5-difluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (33),
3-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (34),
2-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (35),
3-fluorobenzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-3-(4-hydroxyphenyl)propyl]-carbamate (37),
2-fluorobenzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-3-(4-hydroxyphenyl)propyl]-carbamate (38),
2-(2-fluorophenyl)ethyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (40),
2-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (41),
3-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (42),
2-fluorobenzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]-carbamate (43),
3-fluorobenzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]-carbamate (45),
3-methoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (47),
4-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (48),
2-methylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (49),
2,3-dimethoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (50),
2-methoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (51),
(R)-6,8-dichloro-3-{(S)-2-[2-(2-fluorophenyl)ethylamino]-3-methyl-pentanoylamino}-2,3,4,9-tetrahydro-1H-carbazole-3-carboxylic acid ((S)-1-carbamoyl-2-methylbutyl)amide (52),
2-trifluoromethylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (53),
3-trifluoromethylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (54),
3-trifluoromethoxybenzoyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (55),
2-trifluoromethoxybenzoyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (56),
4-fluorobenzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-2-methylbutyl}carbamate (57),
(R)-6,8-dichloro-3-{(S)-2-[2-(4-fluorophenyl)ethylamino]-3-methyl-pentanoylamino}-2,3,4,9-tetrahydro-1H-carbazole-3-carboxylic acid ((S)-1-carbamoyl-2-methylbutyl)amide (58),
(R)-6,8-dichloro-3-{(S)-2-[2-(2,6-difluorophenyl)acetylamino]-3-methyl-pentanoylamino}-2,3,4,9-tetrahydro-1H-carbazole-3-carboxylic acid ((S)-1-carbamoyl-2-methylbutyl)amide (59),
4-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (60),
(R)-6,8-dichloro-3-{(S)-2-[2-(3-fluorophenyl)ethylamino]-3-methyl-pentanoylamino}-2,3,4,9-tetrahydro-1H-carbazole-3-carboxylic acid ((S)-1-carbamoyl-2-methylbutyl)amide (61),
(R)-8-chloro-3-{(S)-2-[2-(2,6-difluorophenyl)acetylamino]-3-methyl-pentanoylamino}-6-fluoro-2,3,4,9-tetrahydro-1H-carbazole-3-carboxylic acid ((S)-2-methyl-1-thiocarbamoylbutyl)amide (62),
(R)-8-chloro-6-fluoro-3-{(S)-2-[2-(4-fluorophenyl)ethylamino]-3-methyl-pentanoylamino}-2,3,4,9-tetrahydro-1H-carbazole-3-carboxylic acid ((S)-1-carbamoyl-2-methylbutyl)amide (63),
4-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (64),
(R)-8-chloro-6-fluoro-3-{(S)-2-[2-(4-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (65),
(R)-8-chloro-3-{(S)-2-[2-(2,4-difluorophenyl)acetylamino]-3-methyl-pentanoylamino}-6-fluoro-2,3,4,9-tetrahydro-1H-carbazole-3-carboxylic acid ((S)-2-methyl-1-thiocarbamoylbutyl)amide (66),
(R)-8-chloro-6-fluoro-3-{(S)-2-[2-(4-fluorophenyl)ethylamino]-3-methyl-pentanoylamino}-2,3,4,9-tetrahydro-1H-carbazole-3-carboxylic acid ((S)-2-methyl-1-thiocarbamoylbutyl)amide (67),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (68),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-1-carbamoyl-2-methylbutyl)amide (69),
(R)-3-{(S)-2-[2-(2-fluorophenyl)acetylamino]-3-methyl-pentanoylamino}-8-trifluoromethyl-2,3,4,9-tetrahydro-1H-carbazole-3-carboxylic acid ((S)-2-cyclopropyl-1-thiocarbamoylethyl)amide (70),
(R)-3-{(S)-2-[2-(2,6-difluorophenyl)acetylamino]-3-methyl-pentanoylamino}-8-trifluoromethyl-2,3,4,9-tetrahydro-1H-carbazole-3-carboxylic acid ((S)-2-cyclopropyl-1-thiocarbamoylethyl)amide (71),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-2-cyclopropyl-1-thiocarbamoylethyl)amide (72),
(R)-3-{(S)-2-[2-(2,6-difluorophenyl)acetylamino]-3-methyl-pentanoylamino}-8-trifluoromethyl-2,3,4,9-tetrahydro-1H-carbazole-3-carboxylic acid ((S)-2-methyl-1-thiocarbamoylbutyl)amide (73),
(R)-3-{(S)-2-[2-(2,6-difluorophenyl)acetylamino]-3-methyl-pentanoylamino}-8-trifluoromethyl-2,3,4,9-tetrahydro-1H-carbazole-3-carboxylic acid ((S)-1-carbamoyl-2-methylbutyl)amide (74),
(R)-3-{(S)-2-[2-(2-fluorophenyl)acetylamino]-3-methyl-pentanoylamino}-8-trifluoromethyl-2,3,4,9-tetrahydro-1H-carbazole-3-carboxylic acid ((S)-1-carbamoyl-2-methylbutyl)amide (75),
(R)-3-{(S)-2-[2-(2-fluorophenyl)acetylamino]-3-methyl-pentanoylamino}-8-trifluoromethyl-2,3,4,9-tetrahydro-1H-carbazole-3-carboxylic acid ((S)-2-methyl-1-thiocarbamoylbutyl)amide (76),
(R)-8-chloro-3-{(S)-2-[2-(2,6-difluorophenyl)acetylamino]-3-methyl-pentanoylamino}-6-fluoro-2,3,4,9-tetrahydro-1H-carbazole-3-carboxylic acid ((S)-2-cyclopropyl-1-thiocarbamoylethyl)amide (77).

Above tetrahydrocarbazole compounds of the formula (I) and tetrahydrocarbazole compounds (1) to (77) are known from WO 2006/005484.

In a further preferred embodiment, at least one LHRH antagonist is provided that can be used for the preparation of a medicament for the herein disclosed treatments, wherein the LHRH antagonist is a tetrahydrocarbazole derivative of the formula (II)

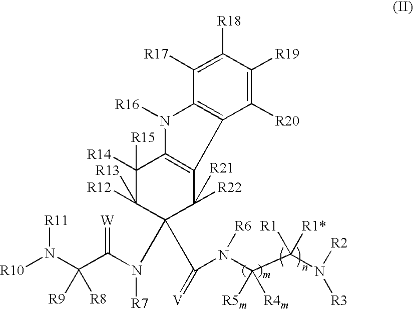

wherein:
(A) V, W are independently from each other selected from the group consisting of: “═O, ═S, ═S⁺—O⁻, germinally linked H₂”;
R1, R1*—when present—together independently form “═O, ═S or ═S⁺—O⁻” or are independently both “hydrogen”;
R2, R3 are independently from each other selected from the group consisting of:
   (i) “hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1, —NX2X3, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)—X4, —C(O)O—X5, —C(O)NH—X6, —C(O)NX7X8, —O—X9, —O(—X10-)ₐ—H (a=1, 2, 3, 4, 5), —O(—X11-O)_{b}—X12 (b=1, 2, 3, 4, 5), —OC(O)X13, —OC(O)O—X14, —OC(O)NHX15, —O—C(O)NX16X17, —OP(O)(OX18)(OX19), —OSi(X20)(X21)(X22), —OS(O₂)—X23, —NHC(O)NH₂, —NHC(O)—X24, —NX25C(O)X26, —NH—C(O)O—X27, —NH—C(O)NH—X28, —NH—C(O)NX29X30, —NX31-C(O)O—X32, —NX33-C(O)—NH—X34, —NX35-C(O)NX36X37, —NHS(O₂)X38, —NX39S(O₂)X40, —S—X41, —S(O)—X42, —S(O₂)X43, —S(O₂)NH—X44, —S(O₂)NX45X46, —S(O₂)O—X47, —P(O)(OX48)(0X49), —Si(X50)(X51)(X52), —C(NH)—NH₂, —C(NX53)NH₂, —C(NH)NHX54, —C(NH)—NX55X56, —C(NX57)NHX58, —C(NX59)-NX60X61, —NH—C(O)NH—O—X62, —NH—C(O)NX63-O—X64, —NX65-C(O)NX66-O—X67, —N(—C(O)—NH—O—X68)₂, —N(—C(O)NX69-O—X70)₂, —N(—C(O)—NH—O—X71)(—C(O)NX72-O—X73), —C(S)—X74, —C(S)O—X75, —C(S)NH—X76, —C(S)—NX77X78, —C(O)NH—O—X79, —C(O)NX80-O—X81, —C(S)NH—O—X82, —C(S)NX83-O—X84, —C(O)—NH—NH—X85, —C(O)—NH—NX86X87, —C(O)—NX88-NX89X90, —C(S)—NH—NH—X91, —C(S)NH—NX92X93, —C(S)NX94-NX95X96, —C(O)—C(O)O—X97, —C(O)—C(O)NH₂, —C(O)C(O)NHX98, —C(O)—C(O)NX99X100, —C(S)—C(O)—O—X101, —C(O)C(S)O—X102, —C(S)—C(S)O—X103, —C(S)C(O)NH₂, —C(S)—C(O) NHX104, —C(S)—C(O)—NX105X106, —C(S)C(S)NH₂, —C(S)—C(S)—NHX107, —C(S)—C(S)NX108X109, —C(O)—C(S)—NH₂, —C(O)C(S)—NHX110, —C(O)C(S)—NX111X112”;
   wherein X1, X2, X3, X4, X5, X6, X7, X8, X9, X10, X11, X12, X13, X14, X15, X16, X17, X18, X19, X20, X21, X22, X23, X24, X25, X26, X27, X28, X29, X30, X31, X32, X33, X34, X35, X36, X37, X38, X39, X40, X41, X42, X43, X44, X45, X46, X47, X48, X49, X50, X51, X52, X53, X54, X55, X56, X57, X58, X59, X60, X61, X62, X63, X64, X65, X66, X67, X68, X69, X70, X71, X72, X73, X74, X75, X76, X77, X78, X79, X80, X81, X82, X83, X84, X85, X86, X87, X88, X89, X90, X91, X92, X93, X94, X95, X96, X97, X98, X99, X100, X100, X102, X103, X104, X105, X106, X107, X108, X109, X110, X111, X112 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X7, X8 and/or X16, X17 and/or X29, X30 and/or X36, X37 and/or X45, X46 and/or X55, X56 and/or X60, X61 and/or X77, X78 and/or X86, X87 and/or X89, X90 and/or X92, X93, and/or X95, X96 and/or X99, X100 and/or X105, X106 and/or X108, X109 and/or X111, X112 and/or respectively together can also form “heterocyclyl”;
   wherein optionally above substituents of substituents group (i) can in turn independently from each other be substituted with at least one substituent, identical or different, selected from the group consisting of:
      (ii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX201, —NX202X203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)—X204, —C(O)O—X205, —C(O)NH—X206, —C(O)NX207X208, —O—X209, —O(—X210-O)_{c}—H (c=1, 2, 3, 4, 5), —O(—X211-O)_{d}—X212 (d=1, 2, 3, 4, 5), —OC(O)—X213, —OC(O)O—X214, —OC(O)NHX215, —O—C(O)NX216X217, —OP(O)(OX218)(0X219), —OSi(X220)(X221)(X222), —OS(O₂)—X223, —NHC(O)—NH₂, —NHC(O)—X224, —NX225C(O)X226, —NH—C(O)O—X227, —NH—C(O)—NH—X228, —NH—C(O)NX229X230, —NX231-C(O)—O—X232, —NX233-C(O)NH—X234, —NX235-C(O)NX236X237, —NHS(O₂)X238, —NX239S(O₂)-X240, —S—X241, —S(O)X242, —S(O₂)X243, —S(O₂)NH—X244, —S(O₂)NX245X246, —S(O₂)O—X247, —P(O)(OX248)(0X249), —Si(X250)(X251)(X252), —C(NH)NH₂, —C(NX253)-NH₂, —C(NH)—NHX254, —C(NH)NX255X256, —C(NX257)NHX258, —C(NX259)—NX260X261, —NH—C(O)NH—O—X262, —NH—C(O)NX263-O—X264, —NX265-C(O)NX266-O—X267, —N(—C(O)—NH—O—X268)₂, —N(—C(O)—NX269-O—X270)₂, —N(—C(O)NH—O—X271)(—C(O)—NX272-O—X273), —C(S)X274, —C(S)O—X275, —C(S)—NH—X276, —C(S)NX277X278, —C(O)NH—O—X279, —C(O)—NX280-O—X281, —C(S)NH—O—X282, —C(S)NX283-O—X284, —C(O)NH—NH—X285, —C(O)NH—NX286X287, —C(O)NX288-NX289X290, —C(S)NH—NH—X291, —C(S)NH—NX292X293, —C(S)NX294-NX295X296, —C(O)C(O)O—X297, —C(O)—C(O)—NH₂, —C(O)C(O)NHX298, —C(O)C(O)NX299X300, —C(S)C(O)O—X301, —C(O)—C(S)—O—X302, —C(S)C(S)O—X303, —C(S)C(O)NH₂, —C(S)C(O)NHX304, —C(S)—C(O)—NX305X306, —C(S)C(S)NH₂, —C(S)C(S)NHX307, —C(S)C(S)NX308X309, —C(O)—C(S)NH₂, —C(O)—C(S)—NHX310, —C(O)—C(S)—NX311X312”;
      wherein X201, X202, X203, X204, X205, X206, X207, X208, X209, X210, X211, X212, X213, X214, X215, X216, X217, X218, X219, X220, X221, X222, X223, X224, X225, X226, X227, X228, X229, X230, X231, X232, X233, X234, X235, X236, X237, X238, X239, X240, X241, X242, X243, X244, X245, X246, X247, X248, X249, X250, X251, X252, X253, X254, X255, X256, X257, X258, X259, X260, X261, X262, X263, X264, X265, X266, X267, X268, X269, X270, X271, X272, X273, X274, X275, X276, X277, X278, X279, X280, X281, X282, X283, X284, X285, X286, X287, X288, X289, X290, X291, X292, X293, X294, X295, X296, X297, X298, X299, X300, X301, X302, X303, X304, X305, X306, X307, X308, X309, X310, X311, X312 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X207, X208 and/or X216, X217 and/or X229, X230 and/or X236, X237 and/or X245, X246 and/or X255, X256 and/or X260, X261 and/or X277, X278 and/or X286, X287 and/or X289, X290 and/or X292, X293 and/or X295, X296 and/or X299, X300 and/or X305, X306 and/or X308, X309 and/or X311, X312 and/or respectively together can also form “heterocyclyl”;
      wherein optionally above substituents of substituents group (ii) can in turn independently from each other be substituted with at least one substituent, identical or different, selected from the group consisting of:
         (iii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX401, —NX402X403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X404, —C(O)O—X405, —C(O)NH—X406, —C(O)NX407X408, —O—X409, —O(—X410-O)ₑ—H (e=1, 2, 3, 4, 5), —O(—X411-O)_{f}-X412 (f=1, 2, 3, 4, 5), —OC(O)X413, —OC(O)—O—X414, —OC(O)—NHX415, —O—C(O)NX416X417, —OP(O)(OX418)(0X419), —OSi(X420)(X421)(X422), —OS(O₂)X423, —NHC(O)—NH₂, —NHC(O)X424, —NX425C(O)X426, —NH—C(O)—O—X427, —NH—C(O)NH—X428, —NH—C(O)NX429X430, —NX431-C(O)O—X432, —NX433-C(O)—NH—X434, —NX435-C(O)—NX436X437, —NHS(O₂)—X438, —NX439S(O₂)X440, —S—X441, —S(O)X442, —S(O₂)X443, —S(O₂)NH—X444, —S(O₂)NX445X446, —S(O₂)O—X447, —P(O)(OX448)(0X449), —Si(X450)(X451)(X452), —C(NH)NH₂, —C(NX453)NH₂, —C(NH)NHX454, —C(NH)—NX455X456, —C(NX457)NHX458, —C(NX459)-NX460X461, —NH—C(O)NH—O—X462, —NH—C(O)NX463-O—X464, —NX465-C(O)—NX466-O—X467, —N(—C(O)—NH—O—X468)₂, —N(—C(O)—NX469-O—X470)₂, —N(—C(O)NH—O—X471)(—C(O)NX472-O—X473), —C(S)—X474, —C(S)—O—X475, —C(S)—NH—X476, —C(S)NX477X478, —C(O)NH—O—X479, —C(O)NX480-O—X481, —C(S)—NH—O—X482, —C(S)NX483-O—X484, —C(O)NH—NH—X485, —C(O)—NH—NX486X487, —C(O)NX488-NX489X490, —C(S)NH—NH—X491, —C(S)NH—NX492X493, —C(S)—NX494-NX495X496, —C(O)—C(O)—O—X497, —C(O)C(O)—NH₂, —C(O)—C(O)—NHX498, —C(O)—C(O)—NX499X500, —C(S)—C(O)O—X501, —C(O)—C(S)—O—X502, —C(S)—C(S)O—X503, —C(S)C(O)NH₂, —C(S)—C(O)NHX504, —C(S)—C(O)NX505X506, —C(S)C(S)—NH₂, —C(S)C(S)NHX507, —C(S)—C(S)—NX508X509, —C(O)—C(S)NH₂, —C(O)—C(S)NHX510, —C(O)—C(S)NX511X512”;
         wherein X401, X402, X403, X404, X405, X406, X407, X408, X409, X410, X411, X412, X413, X414, X415, X416, X417, X418, X419, X420, X421, X422, X423, X424, X425, X426, X427, X428, X429, X430, X431, X432, X433, X434, X435, X436, X437, X438, X439, X440, X441, X442, X443, X444, X445, X446, X447, X448, X449, X450, X451, X452, X453, X454, X455, X456, X457, X458, X459, X460, X461, X462, X463, X464, X465, X466, X467, X468, X469, X470, X471, X472, X473, X474, X475, X476, X477, X478, X479, X480, X481, X482, X483, X484, X485, X486, X487, X488, X489, X490, X491, X492, X493, X494, X495, X496, X497, X498, X499, X500, X501, X502, X503, X504, X505, X506, X507, X508, X509, X510, X511, X512 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X407, X408 and/or X416, X417 and/or X429, X430 and/or X436, X437 and/or X445, X446 and/or X455, X456 and/or X460, X461 and/or X477, X478 and/or X486, X487 and/or X489, X490 and/or X492, X493 and/or X495, X496 and/or X499, X500 and/or X505, X506 and/or X508, X509 and/or X511, X512 and/or respectively together can also form “heterocyclyl”;
n independently is 0 or 1;
with the first proviso that, if R1, R1* are not present (n is 0), R2, R3 must not both be “hydrogen” at the same time;
with the second proviso that, if R1, R1* are present (n is 1) and together independently form “═O, ═S or ═S⁺—O⁻” or are independently both “hydrogen”, R2, R3 must not both be “hydrogen” at the same time;
with the third proviso that, if R1, R1* are not present (n is 0), one of R2, R3 must not be “hydrogen” at the same time when the other one of R2, R3 is “—C(═NH)—NH₂”;
with the fourth proviso that, if R1, R1* are present (n is 1) and are independently both “hydrogen”, one of R2, R3 must not be “hydrogen” at the same time when the other one of R2, R3 is “—C(═NH)—NH₂”;
with the fifth proviso that, if R1, R1* are present (n is 1) and together independently form “═O” and one of R2, R3 independently is “hydrogen” and the other one of R2, R3 independently is “alkyl, cycloalkyl, cycloalkylalkyl, aryl, arylalkyl, heteroaryl”, then the other one of R2, R3 being “alkyl, cycloalkyl, cycloalkylalkyl, aryl, arylalkyl, heteroaryl” must be substituted with at least one substituent selected from the group consisting of:
   (iv) “heterocyclyl, heterocyclylalkyl, —CF3, —N₃, —NH₂, —NHX600, —NX601X602, —NO₂, —OH, —OCF₃, —SH, —O—SO₃H, —OP(O)(OH)₂, —CHO, —COOH, —SO₃H, —P(O)(OH)₂, —C(O)—X603, —C(O)O—X604, —C(O)NH—X605, —C(O)NX606X607, —O-aryl, —O-arylalkyl, —O-heteroaryl, —O-heteroarylalkyl, —O-heterocyclyl, —O-heterocyclylalkyl, —O(—X608-O)_{g}—H (g=1, 2, 3, 4, 5), —O(—X609-O)ₕ—X610 (h=1, 2, 3, 4, 5), —OC(O)—X611, —OC(O)—O—X612, —OC(O)—NHX613, —O—C(O)—NX614X615, —OP(O)(OX616)(0X617), —OSi(X618)(X619)(X620), —OS(O₂)—X621, —NHC(O)X622, —NX623C(O)X624, —NH—C(O)O—X625, —NH—C(O)—NH—X626, —NH—C(O)NX627X628, —NX629-C(O)O—X630, —NX631-C(O)—NH—X632, —NX633-C(O)NX634X635, —NHS(O₂)—X636, —NX637S(O₂)X638, —S—X639, —S(O)—X640, —S(O₂)X641, —S(O₂)NH—X642, —S(O₂)NX643X644, —S(O₂)O—X645, —P(O)(OX646)(0X647), —Si(X648)(X649)(X650), —C(NH)NH₂, —C(NX651)-NH₂, —C(NH)NHX652, —C(NH)NX653X654, —C(NX655)NHX656, —C(NX657)-NX658X659, —NH—C(O)NH—O—X660, —NH—C(O)NX661-O—X662, —NX663-C(O)—NX664-O—X665, —N(—C(O)NH—O—X666)₂, —N(—C(O)—NX667-O—X668)₂, —N(—C(O)—NH—O—X669)(—C(O)NX670-O—X671), —C(S)X672, —C(S)—O—X673, —C(S)NH—X674, —C(S)NX675X676, —C(O)NH—O—X677, —C(O)—NX678-O—X679, —C(S)NH—O—X680, —C(S)NX681-O—X682, —C(O)—NH—NH—X683, —C(O)NH—NX684X685, —C(O)NX686-NX687X688, —C(S)NH—NH—X689, —C(S)NH—NX690X691, —C(S)—NX692-NX693X694, —C(O)C(O)O—X695, —C(O)C(O)—NH₂, —C(O)—C(O)NHX696, —C(O)C(O)—NX697X698, —C(S)—C(O)O—X699, —C(O)—C(S)—O—X700, —C(S)C(S)O—X701, —C(S)—C(O)NH₂, —C(S)—C(O)—NHX702, —C(S)C(O)NX703X704, —C(S)C(S)—NH₂, —C(S)—C(S)—NHX705, —C(S)C(S)NX706X707, —C(O)C(S)—NH₂, —C(O)—C(S)—NHX708, —C(O)C(S)NX709X710”;
   wherein X600, X601, X602, X603, X604, X605, X606, X607, X608, X609, X610, X611, X612, X613, X614, X615, X616, X617, X618, X619, X620, X621, X622, X623, X624, X625, X626, X627, X628, X629, X630, X631, X632, X633, X634, X635, X636, X637, X638, X639, X640, X641, X642, X643, X644, X645, X646, X647, X648, X649, X650, X651, X652, X653, X654, X655, X656, X657, X658, X659, X660, X661, X662, X663, X664, X665, X666, X667, X668, X669, X670, X671, X672, X673, X674, X675, X676, X677, X678, X679, X680, X681, X682, X683, X684, X685, X686, X687, X688, X689, X690, X691, X692, X693, X694, X695, X696, X697, X698, X699, X700, X701, X702, X703, X704, X705, X706, X707, X708, X709, X710, X711, X712 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X606, X607 and/or X614, X615 and/or X627, X628 and/or X634, X635 and/or X643, X644 and/or X653, X654 and/or X658, X659 and/or X675, X676 and/or X684, X685 and/or X687, X688 and/or X690, X691 and/or X693, X694 and/or X697, X698 and/or X703, X704 and/or X706, X707 and/or X709, X710 and/or respectively together can also form “heterocyclyl”;
   with the further proviso that “—C(O)N(alkyl)₂, —C(O)N(cycloalkyl)₂, —C(O)N(cycloalkylalkyl)₂, —C(O)—N(arylalkyl)₂, —C(O)N(aryl)₂, —C(O)—N(heteroaryl)₂” are excluded from above substituents group (iv);
wherein optionally the other one of R2, R3 being “alkyl, cycloalkyl, cycloalkylalkyl, aryl, arylalkyl, heteroaryl” can in turn independently from each other be additionally substituted with at least one substituent, identical or different, selected from above substituents group (ii);
wherein optionally the other one of R2, R3 being “alkyl, cycloalkyl, cycloalkylalkyl, aryl, arylalkyl, heteroaryl” and being substituted with at least one substituent, identical or different, selected from above substituents group (iv) and, optionally, also (ii), can optionally be further substituted in their substituents selected from above substituents group (iv) and, optionally, also (ii), with at least one substituent, identical or different, selected from above substituents group (iii);
with the sixth proviso that, if R1, R1* are present (n is 1) and together independently form “═S or ═S⁺—O” and R2, R3 are independently selected from the group consisting of “hydrogen, alkyl, cycloalkyl, cycloalkylalkyl, aryl, arylalkyl”, each of R2, R3 being “alkyl, cycloalkyl, cycloalkylalkyl, aryl, arylalkyl” must be substituted with at least one substituent selected from the group consisting of:
   (v) “heterocyclyl, heterocyclylalkyl, heteroaryl, heteroarylalkyl, —CF₃, —N₃, —NH₂, —NHX800, —NX801X802, —NO₂, —OH, —OCF₃, —SH, —O—SO₃H, —OP(O)(OH)₂, —CHO, —COOH, —C(O)NH₂, —SO₃H, —P(O)(OH)₂, —C(O)—X803, —C(O)O—X804, —C(O)NH—X805, —C(O)NX806X807, —O-aryl, —O-arylalkyl, —O-heteroaryl, —O-heteroarylalkyl, —O-heterocyclyl, —O-heterocyclylalkyl, —O(—X808-O)ᵣ—H (i=1, 2, 3, 4, 5), —O(—X809-O)F—X810 (j=1, 2, 3, 4, 5), —OC(O)—X811, —OC(O)O—X812, —OC(O)NHX813, —O—C(O)—NX814X815, —OP(O)(OX816)(0X817), —OSi(X818)(X819)(X820), —OS(O₂)—X821, —NHC(O)X822, —NX823C(O)—X824, —NH—C(O)O—X825, —NH—C(O)NH—X826, —NH—C(O)—NX827X828, —NX829-C(O)O—X830, —NX831-C(O)—NH—X832, —NX833-C(O)NX834X835, —NHS(O₂)X836, —NX837S(O₂)—X838, —S—X839, —S(O)X840, —S(O₂)X841, —S(O₂)NH—X842, —S(O₂)NX843X844, —S(O₂)O—X845, —P(O)(OX846)(0X847), —Si(X848)(X849)(X850), —C(NH)—NH₂, —C(NX851)NH₂, —C(NH)NHX852, —C(NH)—NX853X854, —C(NX855)NHX856, —C(NX857)NX858X859, —NH—C(O)NH—O—X860, —NH—C(O)—NX861-O—X862, —NX863-C(O)NX864-O—X865, —N(—C(O)—NH—O—X866)₂, —N(—C(O)NX867-O—X868)₂, —N(—C(O)NH—O—X869)(—C(O)—NX870-O—X871), —C(S)—X872, —C(S)—O—X873, —C(S)NH—X874, —C(S—NX875X876, —C(O)NH—O—X877, —C(O)—NX878-O—X879, —C(S)NH—O—X880, —C(S)NX881-O—X882, —C(O)NH—NH—X883, —C(O)NH—NX884X885, —C(O)NX886-NX887X888, —C(S)—NH—NH—X889, —C(S)NH—NX890X891, —C(S)—NX892-NX893X894, —C(O)—C(O)—O—X895, —C(O)C(O)NH₂, —C(O)—C(O)NHX896, —C(O)—C(O)—NX897X898, —C(S)C(O)O—X899, —C(O)—C(S)—O—X900, —C(S)—C(S)—O—X901, —C(S)C(O)NH₂, —C(S)C(O)NHX902, —C(S)C(O)NX903X904, —C(S)C(S)—NH₂, —C(S)C(S)—NHX905, —C(S)C(S)—NX906X907, —C(O)—C(S)—NH₂, —C(O)—C(S)NHX908, —C(O)—C(S)—NX909X910”;
   wherein X800, X801, X802, X803, X804, X805, X806, X807, X808, X809, X810, X811, X812, X813, X814, X815, X816, X817, X818, X819, X820, X821, X822, X823, X824, X825, X826, X827, X828, X829, X830, X831, X832, X833, X834, X835, X836, X837, X838, X839, X840, X841, X842, X843, X844, X845, X846, X847, X848, X849, X850, X851, X852, X853, X854, X855, X856, X857, X858, X859, X860, X861, X862, X863, X864, X865, X866, X867, X868, X869, X870, X871, X872, X873, X874, X875, X876, X877, X878, X879, X880, X881, X882, X883, X884, X885, X886, X887, X888, X889, X890, X891, X892, X893, X894, X895, X896, X897, X898, X899, X900, X901, X902, X903, X904, X905, X906, X907, X908, X909, X910, X911, X912 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X806, X807 and/or X814, X815 and/or X827, X828 and/or X834, X835 and/or X843, X844 and/or X853, X854 and/or X858, X859 and/or X875, X876 and/or X884, X885 and/or X887, X888 and/or X890, X891 and/or X893, X894 and/or X897, X898 and/or X903, X904 and/or X906, X907 and/or X909, X910 and/or respectively together can also form “heterocyclyl”;
wherein optionally each of R2, R3 being “alkyl, cycloalkyl, cycloalkylalkyl, aryl, arylalkyl” can in turn independently from each other be additionally substituted with at least one substituent, identical or different, selected from above substituents group (ii);
wherein optionally each of R2, R3 being “alkyl, cycloalkyl, cycloalkylalkyl, aryl, arylalkyl” and being substituted with at least one substituent, identical or different, selected from above substituents group (v) and, optionally, also (ii), can optionally be further substituted in their substituents selected from above substituents group (v) and, optionally, also (ii), with at least one substituent, identical or different, selected from above substituents group (iii);
m independently is 1 or 2;
R4ₘ, R5ₘ, R6, R7, R8, R9, R10, R11, R12, R13, R14, R15, R16, R17, R18, R19, R20, R21, R22 are independently from each other selected from the group consisting of:
   (i) “hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1001, —NX1002X1003, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X11004, —C(O)O—X1005, —C(O)NH—X1006, —C(O)NX1007X1008, —O—X1009, —O(—X01 O—O)ₖ—H (k=1, 2, 3, 4, 5), —O(—X1011-O)I—X1012 (I=1, 2, 3, 4, 5), —OC(O)—X1013, —OC(O)O—X1014, —OC(O)—NHX1015, —O—C(O)NX1016X1017, —OP(O)(OX1018)(0X1019), —OSi(X1020)(X1021)(X1022), —OS(O₂)X11023, —NHC(O)—NH₂, —NHC(O)—X1024, —NX1025C(O)—X1026, —NH—C(O)O—X1027, —NH—C(O)—NH—X1028, —NH—C(O)—NX1029X1030, —NX1031-C(O)O—X1032, —NX1033-C(O)NH—X1034, —NX1035-C(O)NX1036X1037, —NHS(O₂)—X1038, —NX1039S(O₂)—X1040, —S—X1041, —S(O)—X1042, —S(O₂)X1043, —S(O₂)NH—X1044, —S(O₂)NX1045X1046, —S(O₂)O—X1047, —P(O)(OX1048)(OX1049), —Si(X1050)(X1051)(X1052), —C(NH)NH₂, —C(NX1053)NH₂, —C(NH)—NHX1054, —C(NH)—NX1055X1056, —C(NX1057)NHX1058, —C(NX1059)-NX1060X1061, —NH—C(O)—NH—O—X1062, —NH—C(O)NX1063-O—X1064, —NX1065-C(O)NX1066-O—X1067, —N(—C(O)NH—O—X1068)₂, —N(—C(O)NX1069-O—X1070)₂, —N(—C(O)NH—O—X1071)(—C(O)NX1072-O—X1073), —C(S)X1074, —C(S)—O—X1075, —C(S)NH—X1076, —C(S)—NX1077X1078, —C(O)—NH—O—X1079, —C(O)NX1080-O—X1081, —C(S)—NH—O—X1082, —C(S)—NX1083-O—X1084, —C(O)NH—NH—X1085, —C(O)NH—NX1086X1087, —C(O)—NX1088-NX1089X1090, —C(S)NH—NH—X1091, —C(S)NH—NX1092X1093, —C(S)—NX1094-NX1095X1096, —C(O)C(O)O—X1097, —C(O)—C(O)—NH₂, —C(O)—C(O)NHX1098, —C(O)—C(O)NX1099X1100, —C(S)—C(O)O—X1101, —C(O)—C(S)—O—X1102, —C(S)C(S)—O—X1103, —C(S)—C(O)NH₂, —C(S)—C(O)—NHX1104, —C(S)C(O)—NX1105X1106, —C(S)C(S)NH₂, —C(S)—C(S)NHX1107, —C(S>C(S)—NX1108X1109, —C(O)C(S)NH₂, —C(O)—C(S)NHX1110, —C(O)—C(S)NX1111 X1112”;
   wherein X1001, X1002, X1003, X1004, X1005, X1006, X1007, X1008, X1009, X1010, X1011, X1012, X1013, X1014, X1015, X1016, X1017, X1018, X1019, X1020, X1021, X1022, X1023, X1024, X1025, X1026, X1027, X1028, X1029, X1030, X1031, X1032, X1033, X1034, X1035, X1036, X1037, X1038, X1039, X1040, X1041, X1042, X1043, X1044, X1045, X1046, X1047, X1048, X1049, X1050, X1051, X1052, X1053, X1054, X1055, X1056, X1057, X1058, X1059, X1060, X1061, X1062, X1063, X1064, X1065, X1066, X1067, X1068, X1069, X1070, X1071, X1072, X1073, X1074, X1075, X1076, X1077, X1078, X1079, X1080, X1081, X1082, X1083, X1084, X1085, X1086, X1087, X1088, X1089, X1090, X1091, X1092, X1093, X1094, X1095, X1096, X1097, X1098, X1099, X1100, X1101, X1102, X1103, X1104, X1105, X1106, X1107, X1108, X1109, X1110, X111, X1112 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X1007, X1008 and/or X1016, X1017 and/or X1029, X1030 and/or X1036, X1037 and/or X1045, X1046 and/or X1055, X1056 and/or X1060, X1061 and/or X1077, X1078 and/or X1086, X1087 and/or X1089, X1090 and/or X1092, X1093 and/or X1095, X1096 and/or X1099, X1100 and/or X1105, X1106 and/or X1108, X1109 and/or X111, X1112 and/or respectively together can also form “heterocyclyl”;
   wherein optionally above substituents of substituents group (i) can in turn independently from each other be substituted with at least one substituent, identical or different, selected from the group consisting of:
   (ii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1201, —NX1202X1203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X1204, —C(O)O—X1205, —C(O)NH—X1206, —C(O)NX1207X1208, —O—X1209, —O(—X1210-0)ₘ—H (m=1, 2, 3, 4, 5), —O(—X1211-0)ₙ—X1212 (n=1, 2, 3, 4, 5), —OC(O)X1213, —OC(O—O—X1214, —OC(O)NHX1215, —O—C(O)NX1216X1217, —OP(O)(OX1218)(OX1219), —OSi(X1220)(X1221)(X1222), —OS(O₂)—X1223, —NHC(O)NH₂, —NHC(O)—X1224, —NX1225C(O)X1226, —NH—C(O)—O—X1227, —NH—C(O)NH—X1228, —NH—C(O)—NX1229X1230, —NX1231-C(O)—O—X1232, —NX1233-C(O)—NH—X1234, —NX1235-C(O)—NX1236X1237, —NHS(O₂)X1238, —NX1239S(O₂)X1240, —S—X1241, —S(O)X1242, —S(O₂)X1243, —S(O₂)NH—X1244, —S(O₂)NX1245X1246, —S(O₂)O—X1247, —P(O)(OX1248)(0X1249), —Si(X1250)(X1251)(X1252), —C(NH)—NH₂, —C(NX1253)NH₂, —C(NH)NHX1254, —C(NH)—NX1255X1256, —C(NX1257)NHX1258, —C(NX1259)NX1260X1261, —NH—C(O)—NH—O—X1262, —NH—C(O)NX1263-O—X1264, —NX1265-C(O)NX1266-O—X1267, —N(—C(O)NH—O—X1268)₂, —N(—C(O)—NX1269-O—X1270)₂, —N(—C(O)NH—O—X1271)(—C(O)NX1272-O—X1273), —C(S>)X1274, —C(S)—O—X1275, —C(S)—NH—X1276, —C(S)—NX1277X1278, —C(O)NH—O—X1279, —C(O)NX1280-O—X1281, —C(S)NH—O—X1282, —C(S)NX1283-O—X1284, —C(O)NH—NH—X1285, —C(O)NH—NX1286X1287, —C(O)—NX1288-NX1289X1290, —C(S)NH—NH—X1291, —C(S)NH—NX1292X11293, —C(S)NX1294-NX1295X1296, —C(O)C(O)—O—X1297, —C(O)—C(O)—NH₂, —C(O)C(O)NHX1298, —C(O)C(O)NX1299X1300, —C(S)—C(O)O—X1301, —C(O)C(S)O—X1302, —C(S)C(S)O—X1303, —C(S)C(O)NH₂, —C(S)C(O)—NHX1304, —C(S)C(O)NX1305X1306, —C(S)—C(S)NH₂, —C(S)—C(S)—NHX1307, —C(S)—C(S)NX1308X1309, —C(O)C(S)NH₂, —C(O)—C(S)—NHX1310, —C(O)C(S)—NX1311X1312”;
   wherein X1201, X1202, X1203, X1204, X1205, X1206, X1207, X1208, X1209, X1210, X1211, X1212, X1213, X1214, X1215, X1216, X1217, X1218, X1219, X1220, X1221, X1222, X1223, X1224, X1225, X1226, X1227, X1228, X1229, X1230, X1231, X1232, X1233, X1234, X1235, X1236, X1237, X1238, X1239, X1240, X1241, X1242, X1243, X1244, X1245, X1246, X1247, X1248, X1249, X1250, X1251, X1252, X1253, X1254, X1255, X1256, X1257, X1258, X1259, X1260, X1261, X1262, X1263, X1264, X1265, X1266, X1267, X1268, X1269, X1270, X1271, X1272, X1273, X1274, X1275, X1276, X1277, X1278, X1279, X1280, X1281, X1282, X1283, X1284, X1285, X1286, X1287, X1288, X1289, X1290, X1291, X1292, X1293, X1294, X1295, X1296, X1297, X1298, X1299, X1300, X1301, X1302, X1303, X1304, X1305, X1306, X1307, X1308, X1309, X1310, X1311, X1312 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X1207, X1208 and/or X1216, X1217 and/or X1229, X1230 and/or X1236, X1237 and/or X1245, X1246 and/or X1255, X1256 and/or X1260, X1261 and/or X1277, X1278 and/or X1286, X1287 and/or X1289, X1290 and/or X1292, X1293 and/or X1295, X1296 and/or X1299, X1300 and/or X1305, X1306 and/or X1308, X1309 and/or X1311, X1312 and/or respectively together can also form “heterocyclyl”;
   wherein optionally above substituents of substituents group (ii) can in turn independently from each other be substituted with at least one substituent, identical or different, selected from the group consisting of:
   (iii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1401, —NX1402X1403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)—X1404, —C(O)O—X1405, —C(O)NH—X1406, —C(O)NX1407X1408, —O—X1409, —O(—X1410-O)ₒ—H (o=1, 2, 3, 4, 5), —O(—X1411-O)ₚ—X1412 (p=1, 2, 3, 4, 5), —OC(O)X1413, —OC(O)O—X1414, —OC(O)—NHX1415, —O—C(O)—NX1416X1417, —OP(O)(OX1418)(0X1419), —OSi(X1420)(X1421)(X1422), —OS(O₂)X1423, —NHC(O)—NH₂, —NHC(O)X1424, —NX1425C(O)X1426, —NH—C(O)O—X1427, —NH—C(O)NH—X1428, —NH—C(O)NX1429X1430, —NX1431-C(O)—O—X1432, —NX1433-C(O)NH—X1434, —NX1435-C(O)—NX1436X1437, —NHS(O₂)X1438, —NX1439S(O₂)X1440, —S—X1441, —S(O)X1442, —S(O₂)X1443, —S(O₂)NH—X1444, —S(O₂)NX1445X1446, —S(O₂)O—X1447, —P(O)(OX1448)(0X1449), —Si(X1450)(X1451)(X1452), —C(NH)NH₂, —C(NX1453)NH₂, —C(NH)—NHX1454, —C(NH)NX1455X1456, —C(NX1457)NHX1458, —C(NX1459)NX1460X1461, —NH—C(O)NH—O—X1462, —NH—C(O)NX1463-O—X1464, —NX1465-C(O)NX1466-O—X1467, —N(—C(O)—NH—O—X1468)₂, —N(—C(O)NX1469-O—X1470)₂, —N(—C(O)—NH—O—X1471)(—C(O)NX1472-O—X1473), —C(S)X1474, —C(S)—O—X1475, —C(S)NH—X1476, —C(S)NX1477X1478, —C(O)NH—O—X1479, —C(O)—NX1480-O—X1481, —C(S)—NH—O—X1482, —C(S)—NX1483-O—X1484, —C(O)NH—NH—X1485, —C(O)—NH—NX1486X1487, —C(O)NX1488-NX1489X1490, —C(S)NH—NH—X1491, —C(S)NH—NX1492X1493, —C(S)NX1494-NX1495X1496, —C(O)C(O)—O—X1497, —C(O)C(O)NH₂, —C(O)C(O)—NHX1498, —C(O)C(O)—NX1499X1500, —C(S)C(O)O—X1501, —C(O)—C(S)—O—X1502, —C(S)—C(S)—O—X1503, —C(S)C(O)—NH₂, —C(S)C(O)—NHX1504, —C(S)C(O)NX1505X1506, —C(S)C(S)NH₂, —C(S)—C(S)NHX1507, —C(S)C(S)NX1508X1509, —C(O)C(S)NH₂, —C(O)C(S)—NHX1510, —C(O)C(S)NX1511X1512”;
   wherein X1401, X1402, X1403, X1404, X1405, X1406, X1407, X1408, X1409, X1410, X1411, X1412, X1413, X1414, X1415, X1416, X1417, X1418, X1419, X1420, X1421, X1422, X1423, X1424, X1425, X1426, X1427, X1428, X1429, X1430, X1431, X1432, X1433, X1434, X1435, X1436, X1437, X1438, X1439, X1440, X1441, X1442, X1443, X1444, X1445, X1446, X1447, X1448, X1449, X1450, X1451, X1452, X1453, X1454, X1455, X1456, X1457, X1458, X1459, X1460, X1461, X1462, X1463, X1464, X1465, X1466, X1467, X1468, X1469, X1470, X1471, X1472, X1473, X1474, X1475, X1476, X1477, X1478, X1479, X1480, X1481, X1482, X1483, X1484, X1485, X1486, X1487, X1488, X1489, X1490, X1491, X1492, X1493, X1494, X1495, X1496, X1497, X1498, X1499, X1500, X1501, X1502, X1503, X1504, X1505, X1506, X1507, X1508, X1509, X1510, X1511, X1512 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X1407, X1408 and/or X1416, X1417 and/or X1429, X1430 and/or X1436, X1437 and/or X1445, X1446 and/or X1455, X1456 and/or X1460, X1461 and/or X1477, X1478 and/or X1486, X1487 and/or X1489, X1490 and/or X1492, X1493 and/or X1495, X1496 and/or X1499, X1500 and/or X1505, X1506 and/or X1508, X1509 and/or X1511, X1512 and/or respectively together can also form “heterocyclyl”;

or
(B) V, W are independently from each other selected from the group consisting of: “═O, ═S, ═S⁺—O⁻, germinally linked H₂”;
R1*, R2 together independently form “heterocyclyl” or together independently form “heteroaryl”; where “heterocyclyl” and “heteroaryl” can optionally be substituted with at least one substituent selected from below substituents group (i);
R1, R3 are independently from each other selected from the group consisting of:
   (i) “hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1, —NZ2Z3, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z4, —C(O)O-Z5, —C(O)NH-Z6, —C(O)NZ7Z8, —O-Z9, —O(-Z10-O)ₐ—H (a=1, 2, 3, 4, 5), —O(-Z1-O)_{b}-Z12 (b=1, 2, 3, 4, 5), —OC(O)-Z13, —OC(O)—O-Z14, —OC(O)NH-Z15, —O—C(O)NZ16Z17, —OP(O)(OZ18)(OZ19), —OSi(Z20)(Z21)(Z22), —OS(O₂)-Z23, —NHC(O)NH₂, —NHC(O)Z24, —NZ25C(O)Z26, —NH—C(O)—O-Z27, —NH—C(O)NH-Z28, —NH—C(O)NZ29Z30, —NZ31-C(O)O-Z32, —NZ33-C(O)—NH-Z34, —NZ35-C(O)NZ36Z37, —NHS(O₂)Z38, —NZ39S(O₂)-Z40, —S-Z41, —S(O)-Z42, —S(O₂)Z43, —S(O₂)NH-Z44, —S(O₂)NZ45Z46, —S(O₂)O-Z47, —P(O)(OZ48)(0Z49), —Si(Z50)(Z51)(Z52), —C(NH)—NH₂, —C(NZ53)-NH₂, —C(NH)—NH-Z54, —C(NH)NZ55Z56, —C(NZ57)NH-Z58, —C(NZ59)—NZ60Z61, —NH—C(O)—NH—O-Z62, —NH—C(O)—NZ63-O-Z64, —NZ65—C(O)NZ66-O-Z67, —N(—C(O)NH—O-Z68)₂, —N(—C(O)NZ69-O-Z70)₂, —N(—C(O)NH—O-Z71)(—C(O)NZ72-O-Z73), —C(S)Z74, —C(S)O-Z75, —C(S)NH-Z76, —C(S)NZ77Z78, —C(O)NH—O-Z79, —C(O)NZ80-O-Z81, —C(S)NH—O-Z82, —C(S)NZ83-O-Z84, —C(O)NH—NH-Z85, —C(O)NH-NZ86Z87, —C(O)NZ88-NZ89Z90, —C(S)—NH—NH-Z91, —C(S)—NH—NZ92Z93, —C(S)NZ94-NZ95Z96, —C(O)—C(O)O-Z97, —C(O)—C(O)NH₂, —C(O)C(O)NH-Z98, —C(O)C(O)—NZ99Z100, —C(S)—C(O)—O-Z101, —C(O)—C(S)O-Z102, —C(S)—C(S)O-Z103, —C(S)C(O)NH₂, —C(S)—C(O)—NH-Z104, —C(S)—C(O)NZ105Z106, —C(S)C(S)NH₂, —C(S)—C(S)—NH-Z107, —C(S)—C(S)NZ108Z109, —C(O)C(S)—NH₂, —C(O)C(S)—NH-Z110, —C(O)C(S)NZ111Z112”;
   wherein Z1, Z2, Z3, Z4, Z5, Z6, Z7, Z8, Z9, Z10, Z11, Z12, Z13, Z14, Z15, Z16, Z17, Z18, Z19, Z20, Z21, Z22, Z23, Z24, Z25, Z26, Z27, Z28, Z29, Z30, Z31, Z32, Z33, Z34, Z35, Z36, Z37, Z38, Z39, Z40, Z41, Z42, Z43, Z44, Z45, Z46, Z47, Z48, Z49, Z50, Z51, Z52, Z53, Z54, Z55, Z56, Z57, Z58, Z59, Z60, Z61, Z62, Z63, Z64, Z65, Z66, Z67, Z68, Z69, Z70, Z71, Z72, Z73, Z74, Z75, Z76, Z77, Z78, Z79, Z80, Z81, Z82, Z83, Z84, Z85, Z86, Z87, Z88, Z89, Z90, Z91, Z92, Z93, Z94, Z95, Z96, Z97, Z98, Z99, Z100, Z101, Z102, Z103, Z104, Z105, Z106, Z107, Z108, Z109, Z10, Z111, Z112 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively Z7, Z8 and/or Z16, Z17 and/or Z29, Z30 and/or Z36, Z37 and/or Z45, Z46 and/or Z55, Z56 and/or Z60, Z61 and/or Z77, Z78 and/or Z86, Z87 and/or Z89, Z90 and/or Z92, Z93 and/or Z95, Z96 and/or Z99, Z100 and/or Z105, Z106 and/or Z108, Z109 and/or Z 111, Z112 and/or respectively together can also form “heterocyclyl”;
   wherein optionally above substituents of substituents group (i) can in turn independently from each other be substituted with at least one substituent, identical or different, selected from the group consisting of:
      (ii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z201, —NZ202Z203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)-Z204, —C(O)O-Z205, —C(O)NH-Z206, —C(O)NZ207Z208, —O-Z209, —O(-Z210-O)ₜ—H (c=1, 2, 3, 4, 5), —O(Z211—O)_{d}-Z212 (d=1,2,3,4,5), —OC(O)-Z213, —OC(O)—O-Z214, —OC(O)—NH-Z215, —O—C(O)—NZ216Z217, —OP(O)(OZ218)(0Z219), —OSi(Z220)(Z221)(Z222), —OS(O₂)Z223, —NHC(O)NH₂, —NHC(O)-Z224, —NZ225C(O)-Z226, —NH—C(O)—O-Z227, —NH—C(O)—NH-Z228, —NH—C(O)NZ229Z230, —NZ231-C(O)—O-Z232, —NZ233-C(O)—NH-Z234, —NZ235-C(O)—NZ236Z237, —NHS(O₂)Z238, —NZ239S(O₂)-Z240, —S-Z241, —S(O)Z242, —S(O₂)-Z243, —S(O₂)NH-Z244, —S(O₂)NZ245Z246, —S(O₂)O-Z247, —P(O)(OZ248)(0Z249), —Si(Z250)(Z251)(Z252), —C(NH)—NH₂, —C(NZ253)NH₂, —C(NH)—NH-Z254, —C(NH)NZ255Z256, —C(NZ257)-NH-Z258, —C(NZ259)—NZ260Z261, —NH—C(O)—NH—O-Z262, —NH—C(O)—NZ263-O-Z264, —NZ265-C(O)—NZ266-O-Z267, —N(—C(O)—NH—O-Z268)₂, —N(—C(O)—NZ269-O-Z270)₂, —N(—C(O)—NH—O-Z271)(—C(O)—NZ272-O-Z273), —C(S)-Z274, —C(S)O-Z275, —C(S)NH-Z276, —C(S)NZ277Z278, —C(O)—NH—O-Z279, —C(O)—NZ280-O-Z281, —C(S)—NH—O-Z282, —C(S)NZ283-O-Z284, —C(O)—NH—NH-Z285, —C(O)NH-NZ286Z287, —C(O)—NZ288-NZ289Z290, —C(S)NH—NH-Z291, —C(S)NH-NZ292Z293, —C(S)NZ294-NZ295Z296, —C(O)—C(O)—O-Z297, —C(O) C(O)—NH₂, —C(O)—C(O)NH-Z298, —C(O)—C(O)NZ299Z300, —C(S)—C(O)—O-Z301, —C(O)C(S)O-Z302, —C(S)C(S)O-Z303, —C(S)—C(O)—NH₂, —C(S)C(O)NH-Z304, —C(S)C(O)—NZ305Z306, —C(S)—C(S)NH₂, —C(S)C(S)NH-Z307, —C(S)—C(S)NZ308Z309, —C(O)—C(S)NH₂, —C(O)—C(S)NH-Z310, —C(O)—C(S)—NZ311Z312”;
      wherein Z201, Z202, Z203, Z204, Z205, Z206, Z207, Z208, Z209, Z210, Z211, Z212, Z213, Z214, Z215, Z216, Z217, Z218, Z219, Z220, Z221, Z222, Z223, Z224, Z225, Z226, Z227, Z228, Z229, Z230, Z231, Z232, Z233, Z234, Z235, Z236, Z237, Z238, Z239, Z240, Z241, Z242, Z243, Z244, Z245, Z246, Z247, Z248, Z249, Z250, Z251, Z252, Z253, Z254, Z255, Z256, Z257, Z258, Z259, Z260, Z261, Z262, Z263, Z264, Z265, Z266, Z267, Z268, Z269, Z270, Z271, Z272, Z273, Z274, Z275, Z276, Z277, Z278, Z279, Z280, Z281, Z282, Z283, Z284, Z285, Z286, Z287, Z288, Z289, Z290, Z291, Z292, Z293, Z294, Z295, Z296, Z297, Z298, Z299, Z300, Z301, Z302, Z303, Z304, Z305, Z306, Z307, Z308, Z309, Z310, Z311, Z312 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively Z207, Z208 and/or Z216, Z217 and/or Z229, Z230 and/or Z236, Z237 and/or Z245, Z246 and/or Z255, Z256 and/or Z260, Z261 and/or Z277, Z278 and/or Z286, Z287 and/or Z289, Z290 and/or Z292, Z293 and/or Z295, Z296 and/or Z299, Z300 and/or Z305, Z306 and/or Z308, Z309 and/or Z311, Z312 and/or respectively together can also form “heterocyclyl”;
      wherein optionally above substituents of substituents group (ii) can in turn independently from each other be substituted with at least one substituent, identical or different, selected from the group consisting of:
         (iii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z401, —NZ402Z403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z404, —C(O)O-Z405, —C(O)NH-Z406, —C(O)NZ407Z408, —O-Z409, —O(-Z410-O)ₑH (e=1, 2, 3, 4, 5), —O(-Z411-O)_{f}Z412 (f=1, 2, 3, 4, 5), —OC(O)-Z413, —OC(O)O-Z414, —OC(O)NH-Z415, —O—C(O)NZ416Z417, —OP(O)(OZ418)(0Z419), —OSi(Z420)(Z421)(Z422), —OS(O₂)Z423, —NHC(O)NH₂, —NHC(O)Z424, —NZ425C(O)-Z426, —NH—C(O)—O-Z427, —NH—C(O)NH-Z428, —NH—C(O)—NZ429Z430, —NZ431-C(O)O-Z432, —NZ433-C(O)NH-Z434, —NZ435-C(O)—NZ436Z437, —NHS(O₂)Z438, —NZ439S(O₂)-Z440, —S-Z441, —S(O)-Z442, —S(O₂)Z443, —S(O₂)NH-Z444, —S(O₂)NZ445Z446, —S(O₂)O-Z447, —P(O)(OZ448)(0Z449), —Si(Z450)(Z451)(Z452), —C(NH)NH₂, —C(NZ453)-NH₂, —C(NH)NH-Z454, —C(NH)—NZ455Z456, —C(NZ457)NH-Z458, —C(NZ459)NZ460Z461, —NH—C(O)NH—O-Z462, —NH—C(O)NZ463-O-Z464, —NZ465-C(O)—NZ466-O-Z467, —N(—C(O)NH—O-Z468)₂, —N(—C(O)NZ469-O-Z470)₂, —N(—C(O)—NH—O-Z471)(—C(O)NZ472-O-Z473), —C(S)-Z474, —C(S)O-Z475, —C(S)—NH-Z476, —C(S)NZ477Z478, —C(O)—NH—O-Z479, —C(O)—NZ480-O-Z481, —C(S)—NH—O-Z482, —C(S)—NZ483-O-Z484, —C(O)NH—NH-Z485, —C(O)—NH-NZ486Z487, —C(O)—NZ488-NZ489Z490, —C(S)—NH—NH-Z491, —C(S)—NH—NZ492Z493, —C(S)NZ494-NZ495Z496, —C(O)—C(O)—O-Z497, —C(O)—C(O)—NH₂, —C(O)—C(O)—NH-Z498, —C(O)C(O)—NZ499Z500, —C(S)C(O)—O-Z501, —C(O)—C(S)O-Z502, —C(S)C(S)O-Z503, —C(S)C(O)—NH₂, —C(S)—C(O)—NH-Z504, —C(S)C(O)—NZ505Z506, —C(S)C(S)NH₂, —C(S)—C(S)—NH-Z507, —C(S)C(S)—NZ508Z509, —C(O)—C(S)NH₂, —C(O)—C(S)NH-Z510, —C(O)—C(S)—NZ511Z512”;
         wherein Z401, Z402, Z403, Z404, Z405, Z406, Z407, Z408, Z409, Z410, Z411, Z412, Z413, Z414, Z415, Z416, Z417, Z418, Z419, Z420, Z421, Z422, Z423, Z424, Z425, Z426, Z427, Z428, Z429, Z430, Z431, Z432, Z433, Z434, Z435, Z436, Z437, Z438, Z439, Z440, Z441, Z442, Z443, Z444, Z445, Z446, Z447, Z448, Z449, Z450, Z451, Z452, Z453, Z454, Z455, Z456, Z457, Z458, Z459, Z460, Z461, Z462, Z463, Z464, Z465, Z466, Z467, Z468, Z469, Z470, Z471, Z472, Z473, Z474, Z475, Z476, Z477, Z478, Z479, Z480, Z481, Z482, Z483, Z484, Z485, Z486, Z487, Z488, Z489, Z490, Z491, Z492, Z493, Z494, Z495, Z496, Z497, Z498, Z499, Z500, Z501, Z502, Z503, Z504, Z505, Z506, Z507, Z508, Z509, Z510, Z511, Z512 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively Z407, Z408 and/or Z416, Z417 and/or Z429, Z430 and/or Z436, Z437 and/or Z445, Z446 and/or Z455, Z456 and/or Z460, Z461 and/or Z477, Z478 and/or Z486, Z487 and/or Z489, Z490 and/or Z492, Z493 and/or Z495, Z496 and/or Z499, Z500 and/or Z505, Z506 and/or Z508, Z509 and/or Z511, Z512 and/or respectively together can also form “heterocyclyl”;
alternatively, R1, R3 can also independently from each other be “no substituent”;
n independently is 1;
m independently is 1 or 2;
R4ₘ, R5ₘ, R6, R7, R8, R9, R10, R11, R12, R13, R14, R15, R16, R17, R18, R19, R20, R21, R22 are independently from each other selected from the group consisting of:
   (i) “hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1001, —NZ1002Z1003, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)-Z1004, —C(O)O-Z1005, —C(O)NH-Z1006, —C(O)NZ1007Z1008, —O-Z1009, —O(-Z1010-O)ₖ—H (k=1, 2, 3, 4, 5), —O(-Z1011—O)Z-Z1012 (I=1,2,3,4,5), —OC(O)-Z1013, —OC(O)—O-Z1014, —OC(O)—NH-Z1015, —O—C(O)—NZ1016Z1017, —OP(O)(OZ1018)(OZ1019), —OSi(Z1020)(Z1021)(Z1022), —OS(O₂)-Z1023, —NHC(O)—NH₂, —NHC(O)-Z1024, —NZ1025C(O-Z1026, —NH—C(O)—O-Z1027, —NH—C(O)—NH-Z1028, —NH—C(O)—NZ1029Z1030, —NZ1031-C(O)—O-Z1032, —NZ1033-C(O)—NH-Z1034, —NZ1035-C(O)—NZ1036Z1037, —NHS(O₂)-Z1038, —NZ1039S(O₂)-Z1040, —S-Z1041, —S(O)-Z1042, —S(O₂)-Z1043, —S(O₂)NH-Z1044, —S(O₂)NZ1045Z1046, —S(O₂)O-Z1047, —P(O)(OZ1048)(OZ1049), —Si(Z1050)(Z1051)(Z1052), —C(NH)—NH₂, —C(NZ1053)-NH₂, —C(NH)—NH-Z1054, —C(NH)—NZ1055Z1056, —C(NZ1057)—NH-Z1058, —C(NZ1059)-NZ1060Z1061, —NH—C(O)—NH—O-Z1062, —NH—C(O)—NZ1063-O-Z1064, —NZ1065-C(O)—NZ1066-O-Z1067, —N(—C(O)—NH—O-Z1068)₂, —N(—C(O)—NZ1069-O-Z1070)₂, —N(—C(O)—NH—O-Z1071)(—C(O)—NZ1072-O-Z1073), —C(S)-Z1074, —C(S)—O-Z1075, —C(S)—NH-1076, —C(S)—NZ1077Z1078, —C(O)—NH—O-Z1079, —C(O)—NZ1080—O-Z Z081, —C(S)—NH—O-Z1082, —C(S)—NZ1083-O-Z1084, —C(O)—NH—NH-Z1085, —C(O)—NH-NZ1086Z1087, —C(O)—NZ1088-NZ1089Z1090, —C(S)—NH—NH-Z1091, —C(S)—NH-NZ1092Z1093, —C(S)—NZ1094-NZ1095Z1096, —C(O)—C(O)—O-Z1097, —C(O)—C(O)—NH₂, —C(O)—C(O)—NH-Z1098, —C(O)—C(O)—NZ1099Z1100, —C(S)—C(O)—O-Z1101, —C(O)—C(S)—O-Z1102, —C(S)—C(S)—O-Z1103, —C(S)—C(O)—NH₂, —C(S)—C(O)—NH-Z1104, —C(S)—C(O)—NZ1105Z1106, —C(S)—C(S)—NH₂, —C(S)—C(S)—NH-Z1107, —C(S)—C(S)—NZ1108Z1109, —C(O)—C(S)—NH₂, —C(O)—C(S)—NH-Z1110, —C(O)—C(S-NZ1111Z1112”;
   wherein X1001, X1002, X1003, X1004, X1005, X1006, X1007, X1008, X1009, X1010, X1011, X1012, X1013, X1014, X1015, X1016, X1017, X1018, X1019, X1020, X1021, X1022, X1023, X1024, X1025, X1026, X1027, X1028, X1029, X1030, X1031, X1032, X1033, X1034, X1035, X1036, X1037, X1038, X1039, X1040, X1041, X1042, X1043, X1044, X1045, X1046, X1047, X1048, X1049, X1050, X1051, X1052, X1053, X1054, X1055, X1056, X1057, X1058, X1059, X1060, X1061, X1062, X1063, X1064, X1065, X1066, X1067, X1068, X1069, X1070, X1071, X1072, X1073, X1074, X1075, X1076, X1077, X1078, X1079, X1080, X1081, X1082, X1083, X1084, X1085, X1086, X1087, X1088, X1089, X1090, X1091, X1092, X1093, X1094, X1095, X1096, X1097, X1098, X1099, X1100, X1011, X1102, X1103, X1104, X1105, X1106, X1107, X1108, X1109, X1110, X111, X1112 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively X1007, X1008 and/or X1016, X1017 and/or X1029, X1030 and/or X1036, X1037 and/or X1045, X1046 and/or X1055, X1056 and/or X1060, X1061 and/or X1077, X1078 and/or X1086, X1087 and/or X1089, X1090 and/or X1092, X1093 and/or X1095, X1096 and/or X1099, X1100 and/or X1105, X1106 and/or X1108, X1109 and/or X1111, X1112 and/or respectively together can also form “heterocyclyl”;
   wherein optionally above substituents of substituents group (i) can in turn independently from each other be substituted with at least one substituent, identical or different, selected from the group consisting of:
      (ii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1201, —NZ1202Z1203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z1204, —C(O)O-Z1205, —C(O)NH-Z1206, —C(O)NZ1207Z1208, —O-Z1209, —O(-Z1210-O)ₘ—H (m=1, 2, 3, 4, 5), —O(-Z1211-O)ₙ-Z 1212 (n=1, 2, 3, 4, 5), —OC(O)Z1213, —OC(O)O-Z1214, —OC(O)NH-Z1215, —O—C(O)NZ1216Z1217, —OP(O)(OZ1218)(OZ1219), —OSi(Z1220)(Z1221)(Z1222), —OS(O₂)-Z1223, —NHC(O)NH₂, —NHC(O)-Z1224, —NZ1225C(O)-Z1226, —NH—C(O)—O-Z1227, —NH—C(O)NH-Z1228, —NH—C(O)—NZ1229Z1230, —NZ1231-C(O)O-Z1232, —NZ1233-C(O)NH-Z1234, —NZ1235-C(O)—NZ1236Z1237, —NHS(O₂)-Z1238, —NZ1239S(O₂-Z1240, —S-Z1241, —S(O)-Z1242, —S(O₂)-Z1243, —S(O₂)NH-Z1244, —S(O₂)NZ1245Z1246, —S(O₂)O-Z1247, —P(O)(OZ1248)(OZ1249), —Si(Z1250)(Z1251)(Z1252), —C(NH)—NH₂, —C(NZ1253)-NH₂, —C(NH)—NH-Z1254, —C(NH)—NZ1255Z1256, —C(NZ1257)-NH-Z1258, —C(NZ1259)-NZ1260Z1261, —NH—C(O)—NH—O-Z1262, —NH—C(O)—NZ1263-O-Z1264, —NZ1265-C(O)—NZ1266-O-Z1267, —N(—C(O)—NH—O-Z1268)₂, —N(—C(O)—NZ1269-O-Z1270)₂, —N(—C(O)—NH—O-Z1271)(—C(O)—NZ1272-O-Z1273), —C(S-Z1274, —C(S)—O-Z1275, —C(S)—NH-Z1276, —C(S)—NZ1277Z1278, —C(O)—NH—O-Z1279, —C(O)—NZ1280-O-Z1281, —C(S)—NH—O-Z1282, —C(S)—NZ1283-O-Z1284, —C(O)—NH—NH-Z1285, —C(O)—NH-NZ1286Z1287, —C(O)—NZ1288-NZ1289Z1290, —C(S)—NH—NH-Z1291, —C(S)—NH-NZ1292Z1293, —C(S)—NZ1294-NZ1295Z1296, —C(O)—C(O)—O-Z1297, —C(O)—C(O)—NH₂, —C(O)—C(O)—NH-Z1298, —C(O)—C(O)—NZ1299Z1300, —C(S)—C(O)—O-Z1301, —C(O)—C(S)—O-Z1302, —C(S)—C(S)—O-Z1303, —C(S)—C(O)—NH₂, —C(S)—C(O)—NH-Z1304, —C(S)—C(O)—NZ1305Z1306, —C(S)—C(S)—NH₂, —C(S)—C(S)—NH-Z1307, —C(S)—C(S)—NZ1308Z1309, —C(O)—C(S)—NH₂, —C(O)—C(S)—NH-Z1310, —C(O—C(S-NZ1311Z1312”;
      wherein Z1201, Z1202, Z1203, Z1204, Z1205, Z1206, Z1207, Z1208, Z1209, Z1210, Z1211, Z1212, Z1213, Z1214, Z1215, Z1216, Z1217, Z1218, Z1219, Z1220, Z1221, Z1222, Z1223, Z1224, Z1225, Z1226, Z1227, Z1228, Z1229, Z1230, Z1231, Z1232, Z1233, Z1234, Z1235, Z1236, Z1237, Z1238, Z1239, Z1240, Z1241, Z1242, Z1243, Z1244, Z1245, Z1246, Z1247, Z1248, Z1249, Z1250, Z1251, Z1252, Z1253, Z1254, Z1255, Z1256, Z1257, Z1258, Z1259, Z1260, Z1261, Z1262, Z1263, Z1264, Z1265, Z1266, Z1267, Z1268, Z1269, Z1270, Z1271, Z1272, Z1273, Z1274, Z1275, Z1276, Z1277, Z1278, Z1279, Z1280, Z1281, Z1282, Z1283, Z1284, Z1285, Z1286, Z1287, Z1288, Z1289, Z1290, Z1291, Z1292, Z1293, Z1294, Z1295, Z1296, Z1297, Z1298, Z1299, Z1300, Z1301, Z1302, Z1303, Z1304, Z1305, Z1306, Z1307, Z1308, Z1309, Z1310, Z1311, Z1312 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively Z1207, Z1208 and/or Z1216, Z1217 and/or Z1229, Z1230 and/or Z1236, Z1237 and/or Z1245, Z1246 and/or Z1255, Z1256 and/or Z1260, Z1261 and/or Z1277, Z1278 and/or Z1286, Z1287 and/or Z1289, Z1290 and/or Z1292, Z1293 and/or Z1295, Z1296 and/or Z1299, Z1300 and/or Z1305, Z1306 and/or Z1308, Z1309 and/or Z1311, Z1312 and/or respectively together can also form “heterocyclyl”;
      wherein optionally above substituents of substituents group (ii) can in turn independently from each other be substituted with at least one substituent, identical or different, selected from the group consisting of:
         (iii) “alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1401, —NZ1402Z1403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z1404, —C(O)O-Z1405, —C(O)NH-Z1406, —C(O)NZ1407Z1408, —O-Z1409, —O(-Z1410-O)ₒ—H (o=1, 2, 3, 4, 5), —O(-Z1411-O)ₚ-Z1412 (p=1, 2, 3, 4, 5), —OC(O)-Z1413, —OC(O)O-Z1414, —OC(O)NH-Z1415, —O—C(O)—NZ1416Z1417, —OP(O)(OZ1418)(OZ1419), OSi(Z1420)(Z1421)(Z1422), —OS(O₂)-Z1423, —NHC(O)—NH₂, —NHC(O)-Z1424, —NZ1425C(O)-Z1426, —NH—C(O)O-Z1427, —NHC(O)NH-Z1428, —NH—C(O)NZ1429Z1430, —NZ1431-C(O)—O-Z1432, —NZ1433-C(O)NH-Z1434, —NZ1435-C(O)NZ1436Z1437, —NHS(O₂)Z1438, —NZ1439S(O₂)-Z1440, —S-Z1441, —S(O)-Z1442, —S(O₂)Z1443, —S(O₂)NH-Z1444, —S(O₂)NZ1445Z1446, —S(O₂)OZ1447, —P(O)(OZ1448)(OZ1449), —Si(Z1450)(Z1451)(Z1452), —C(NH)—NH₂, —C(NZ1453)NH₂, —C(NH)NH-Z1454, —C(NH)—NZ1455Z1456, —C(NZ1457)-NH-Z1458, —C(NZ1459)—NZ1460Z1461, —NH—C(O)—NH—O-Z1462, —NH—C(O)NZ1463-O-Z1464, —NZ1465-C(O)—NZ1466-O-Z1467, —N(—C(O)13 NH—O-Z1468)₂, —N(—C(O)NZ1469-O-Z1470)₂, —N(—C(O)—NH—O-Z1471)(—C(O)NZ1472-O-Z1473), —C(S)-Z1474, —C(S)—O-Z1475, —C(S)NH-Z1476, —C(S)NZ1477Z1478, —C(O)NH—O-Z1479, —C(O)NZ1480-O-Z1481, —C(S)—NH—O-Z1482, —C(S)—NZ1483-O-Z1484, —C(O)NH—NH-Z1485, —C(O)NH-NZ1486Z1487, —C(O)—NZ1488-NZ1489Z1490, —C(S)—NH—NH-Z1491, —C(S)—NH—NZ1492Z1493, —C(S)—NZ1494-NZ1495Z1496, —C(O)—C(O)—O-Z1497, —C(O)—C(O)—NH₂, —C(O)—C(O)—NH-Z1498, —C(O)—C(O)—NZ1499Z1500, —C(S)—C(O)—O-Z1501, —C(O)—C(S)—O-Z1502, —C(S)—C(S)—O-Z1503, —C(S)—C(O)—NH₂, —C(S)—C(O)—NH-Z1504, —C(S)—C(O)—NZ1505Z1506, —C(S)—C(S)—NH₂, —C(S)—C(S)—NH-Z1507, —C(S)—C(S)—NZ1508Z1509, —C(O)—C(S)—NH₂, —C(O)C(S)—NH-Z1510, —C(O)—C(S)—NZ1511Z1512”;
         wherein Z1401, Z1402, Z1403, Z1404, Z1405, Z1406, Z1407, Z1408, Z1409, Z1410, Z1411, Z1412, Z1413, Z1414, Z1415, Z1416, Z1417, Z1418, Z1419, Z1420, Z1421, Z1422, Z1423, Z1424, Z1425, Z1426, Z1427, Z1428, Z1429, Z1430, Z1431, Z1432, Z1433, Z1434, Z1435, Z1436, Z1437, Z1438, Z1439, Z1440, Z1441, Z1442, Z1443, Z1444, Z1445, Z1446, Z1447, Z1448, Z1449, Z1450, Z1451, Z1452, Z1453, Z1454, Z1455, Z1456, Z1457, Z1458, Z1459, Z1460, Z1461, Z1462, Z1463, Z1464, Z1465, Z1466, Z1467, Z1468, Z1469, Z1470, Z1471, Z1472, Z1473, Z1474, Z1475, Z1476, Z1477, Z1478, Z1479, Z1480, Z1481, Z1482, Z1483, Z1484, Z1485, Z1486, Z1487, Z1488, Z1489, Z1490, Z1491, Z1492, Z1493, Z1494, Z1495, Z1496, Z1497, Z1498, Z1499, Z1500, Z1501, Z1502, Z1503, Z1504, Z1505, Z1506, Z1507, Z1508, Z1509, Z1510, Z1511, Z1512 are independently from each other selected from the group consisting of: “alkyl, (C₉-C₃₀)alkyl, cycloalkyl, cycloalkylalkyl, heterocyclyl, heterocyclylalkyl, aryl, arylalkyl, heteroaryl, heteroarylalkyl” and wherein alternatively Z1407, Z1408 and/or Z1416, Z1417 and/or Z1429, Z1430 and/or Z1436, Z1437 and/or Z1445, Z1446 and/or Z1455, Z1456 and/or Z1460, Z1461 and/or Z1477, Z1478 and/or Z1486, Z1487 and/or Z1489, Z1490 and/or Z1492, Z1493 and/or Z1495, Z1496 and/or Z1499, Z1500 and/or Z1505, Z1506 and/or Z1508, Z1509 and/or Z1511, Z1512 and/or respectively together can also form “heterocyclyl”.

With regard to the alternative embodiment “no substituent” for R1 and R3, it is understood in the course of the present invention that R1 and/or R3 are not present and that the valences of the respective carbon and/or nitrogen atom, of which R1 and R3 are ligands and that are part of “heterocyclyl” or “heteroaryl”, are fully used up by means of double and/or triple bonds.

With regard to R1, R1* and n, it is understood in the course of the present invention that if n is 0 substituents R1, R1* and the corresponding harbouring carbon atom are not present, i.e. the nitrogen atom harbouring R2, R3 is directly attached to the carbon atom harbouring R4ₘ, R5ₘ. If n is 1, then one carbon atom harbouring R1, R1* is present between the carbon atom harbouring R4ₘ, R5ₘ and the nitrogen atom harbouring R2, R3.

With regard to R4ₘ, R5ₘ, and m, it is understood in the course of the present invention that if m is 1, one carbon atom harbouring one radical R4ₘ and one radical R5ₘ is present. If m is 2, then two carbon atoms each harbouring one radical R4ₘ and one radical R5ₘ are present, where all four radicals R4ₘ₁, R5ₘ₁, R4ₘ₂, R5ₘ₂ can independently from each other be identical or different.

In a further preferred embodiment, at least one LHRH antagonist is provided that can be used for the preparation of a medicament for the herein disclosed treatments, wherein the LHRH antagonist is selected from the group consisting of:

### Compound 1 ((S)-1-{(R)-3-[(R)-1-(5-Amino-[1,3,4]oxadiazol-2-yl)-2-methylbutyl-carbamoyl]-6,8-dichloro-2,3,4,9-tetrahydro-1H-carbazol-3-ylcarbamoyl}-2-methyl-butyl)-carbamic acid benzyl ester

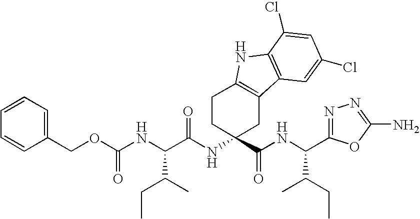

### Compound 2 ((S)-1-{(S)-3-[(R)-1-(5-Amino-[1,3,4]oxadiazol-2-yl)-2-methylbutyl-carbamoyl]-6,8-dichloro-2,3,4,9-tetrahydro-1H-carbazol-3-ylcarbamoyl}-2-methyl-butyl)-carbamic acid benzyl ester

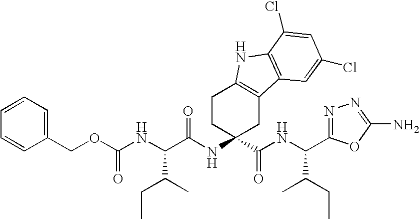

### Compound 3 ((S)-1-{(R)-6,8-Dichloro-3-[(S)-2-methyl-1-(3-methyl-[1,2,4]oxadiazol-5-yl)-butylcarbamoyl]-2,3,4,9-tetrahydro-1H-carbazol-3-ylcarbamoyl}-2-methyl-butyl)-carbamic acid benzyl ester

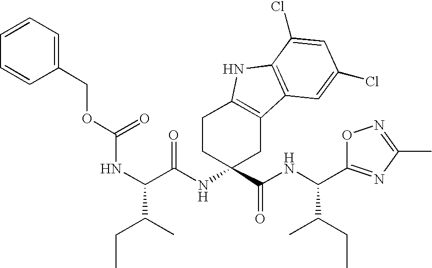

### Compound 4 ((S)-1-{(S)-6,8-Dichloro-3-[(S)-2-methyl-1-(3-methyl-[1,2,4]oxadiazol-5-yl)-butylcarbamoyl]-2,3,4,9-tetrahydro-1H-carbazol-3-ylcarbamoyl}-2-methyl-butyl)-carbamic acid benzyl ester

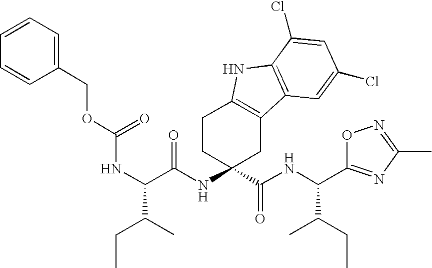

### Compound 5 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid ethyl ester

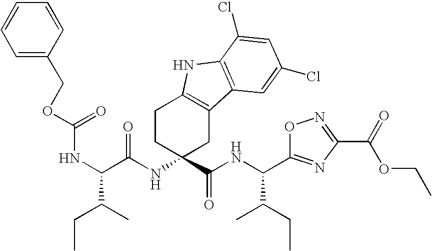

### Compound 6 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid ethyl ester

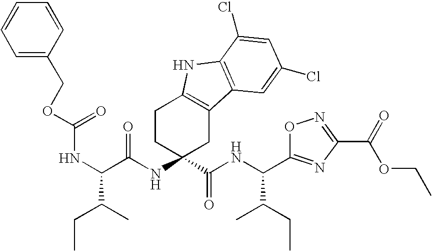

### Compound 7 ((S)-1-{(S)-6,8-Dichloro-3-[(S)-2-methyl-1-(5-oxo-4,5-dihydro-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester

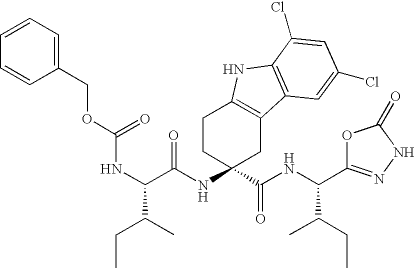

### Compound 8 {(S)-1-[(R)-6,8-Dichloro-3-((S)-2-methyl-1-[1,3,4]oxadiazol-2-yl-butylcarbamoyl)-2,3,4,9-tetrahydro-1H-carbazol-3-ylcarbamoyl]-2-methyl-butyl}-carbamic acid benzyl ester

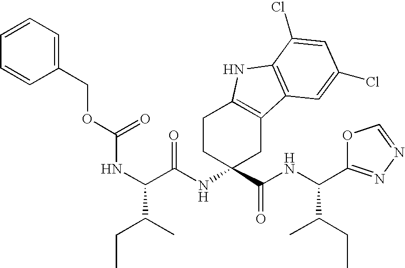

### Compound 9 {(S)-1-[(S)-6,8-Dichloro-3-((S)-2-methyl-1-[1,3,4]oxadiazol-2-yl-butylcarbamoyl)-2,3,4,9-tetrahydro-1H-carbazol-3-ylcarbamoyl]-2-methyl-butyl}-carbamic acid benzyl ester

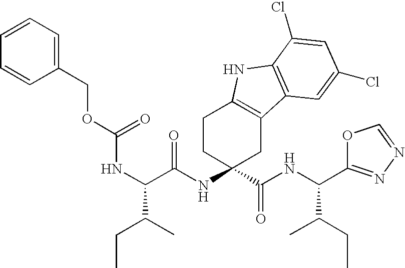

### Compound 10 ((S)-1-{(R)-3-[(S)-1-(3-Carbamoyl-[1,2,4]oxadiazol-5-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester

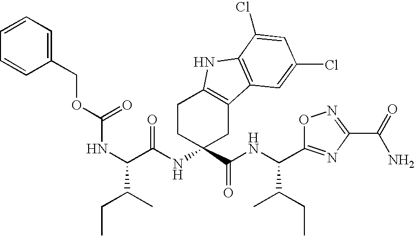

### Compound 11 ((S)-1-{(S)-3-[(S)-1-(3-Carbamoyl-[1,2,4]oxadiazol-5-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester

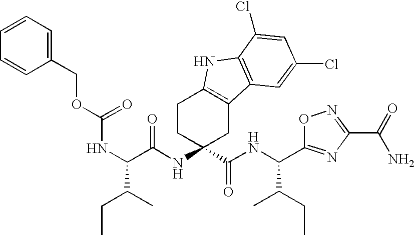

### Compound 12 5-{(S)-1-[((R)-3-{(S)-2-[2-(2,6-Difluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester

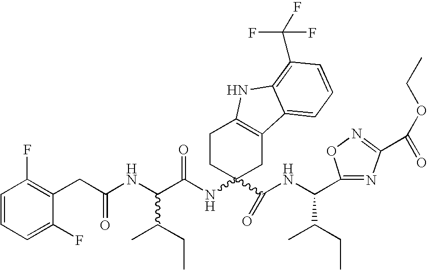

### Compound 13 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl)-amino}-2-methyl-butyl)-[1,3,4]oxadiazole-2-carboxylic acid ethyl ester

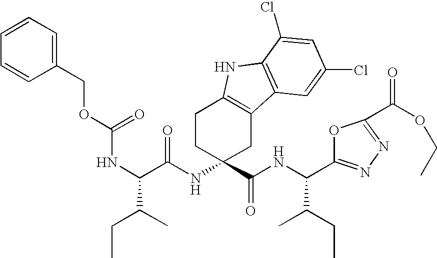

### Compound 14 ((S)-1-{(R)-3-[(S)-1-(5-Acetylamino-[1,3,4]oxadiazol-2-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester

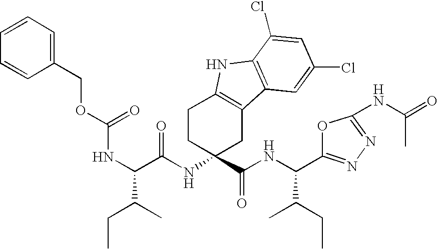

### Compound 15 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,3,4]oxadiazole-2-carboxylic acid ethyl ester

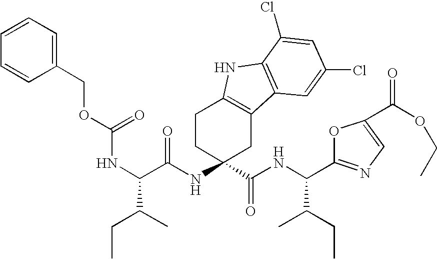

### Compound 16 ((S)-1-{(S)-3-[(S)-1-(5-Acetylamino-[1,3,4]oxadiazol-2-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester

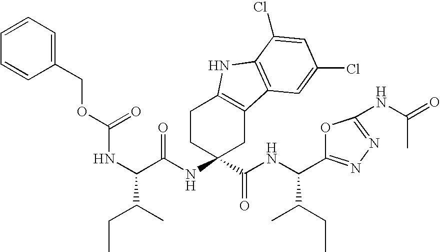

### Compound 17 ((S)-1-{(R)-6,8-Dichloro-3-[(S)-2-methyl-1-(5-oxo-4,5-dihydro-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester

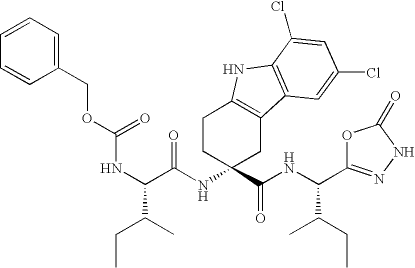

### Compound 18 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid propyl ester

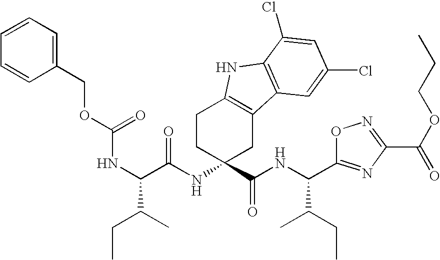

### Compound 19 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid propyl ester

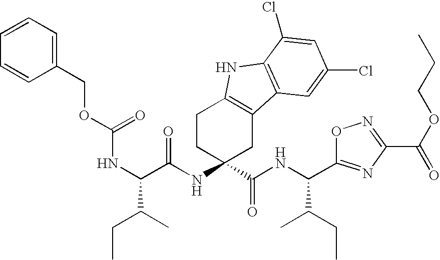

### Compound 20 ((S)-1-{(R)-6,8-Dichloro-3-[(S)-1-(3-diethylcarbamoyl-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester

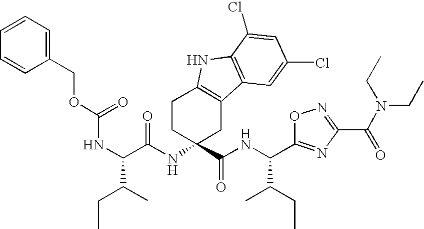

### Compound 21 ((S)-1-{(R)-6,8-Dichloro-3-[(S)-1-(3-cyano-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester

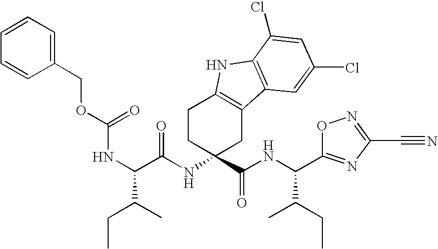

### Compound 22 ((S)-1-{(S)-6,8-Dichloro-3-[(S)-1-(3-cyano-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester

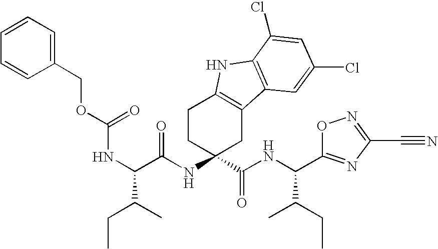

### Compound 23 ((S)-1-{(R)-6,8-Dichloro-3-[(S)-2-methyl-1-(5-methyl-[1,3,4]oxadiazol-2-yl)-butylcarbamoyl]-2,3,4,9-tetrahydro-1H-carbazol-3-ylcarbamoyl}-2-methyl-butyl)-carbamic acid benzylester

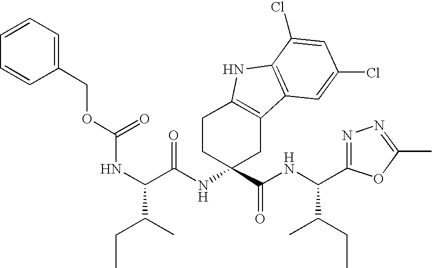

### Compound 24 ((S)-1-{(S)-6,8-Dichloro-3-[(S)-2-methyl-1-(5-methyl-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester

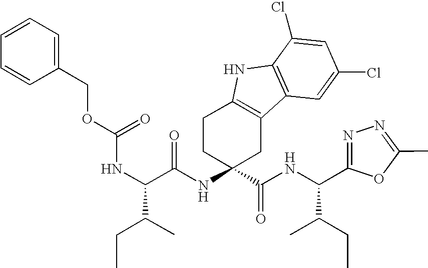

### Compound 255-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid methyl ester

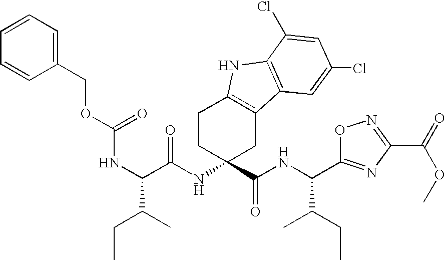

### Compound 26 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid methyl ester

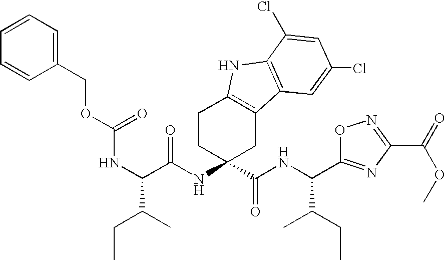

### Compound 27 [(S)-1-((R)-6,8-Dichloro-3-{(S)-1-[5-(3-ethyl-ureido)-[1,3,4,]oxadiazol-2-yl]-2-methyl-butylcarbamoyl}-2,3,4,9-tetrahydro-1H-carbazol-3-ylcarbamoyl)-2-methyl-butyl]-carbamic acid benzyl ester

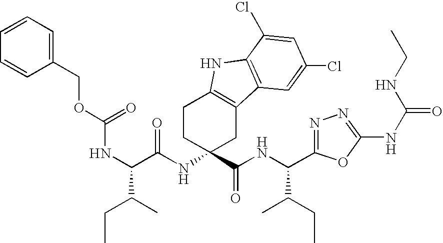

### Compound 28 5-{(S)-1-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester

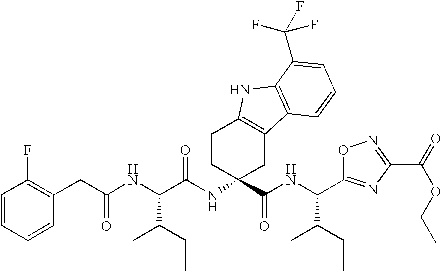

### Compound 29 5-{(S)-1-[((S)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester

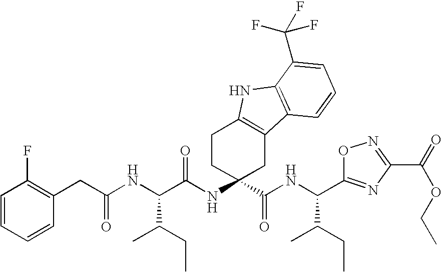

### Compound 30 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide

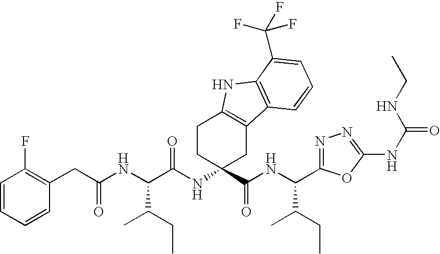

### Compound 31 (S)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide

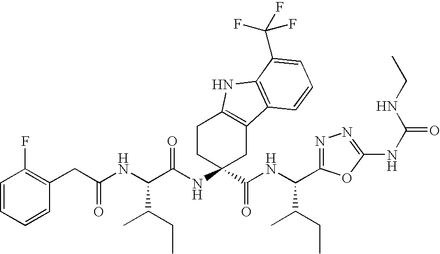

### Compound 32 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(5-amino-[1,3,4]oxadiazol-2-yl)-2-methyl-butyl]-amide

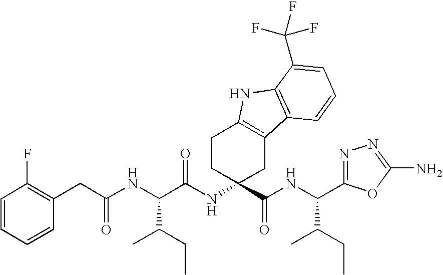

### Compound 33

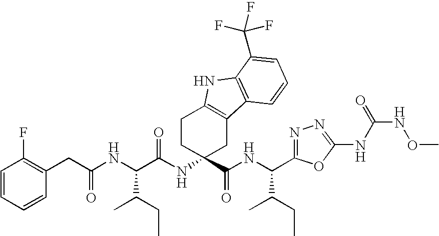

### Compound 34

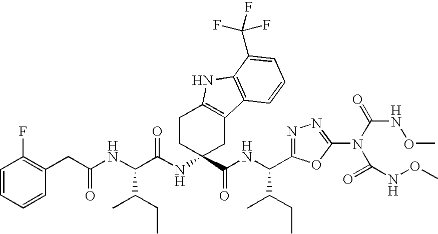

### Compound 35 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrrolidin-1-ylmethyl)-carbamoyl]-butyl}-amide

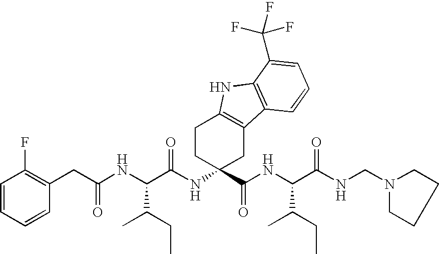

### Compound 36 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(morpholin-4-ylmethyl)-carbamoyl]-butyl}-amide

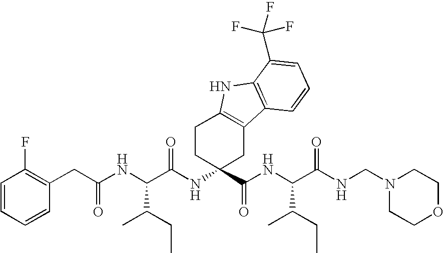

### Compound 37 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(morpholin-4-ylmethyl)-thiocarbamoyl]-butyl}-amide

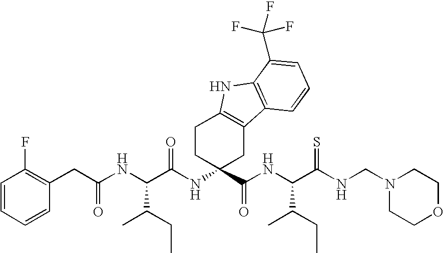

### Compound 38 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-methoxycarbamoyl-2-methylbutyl)-amide

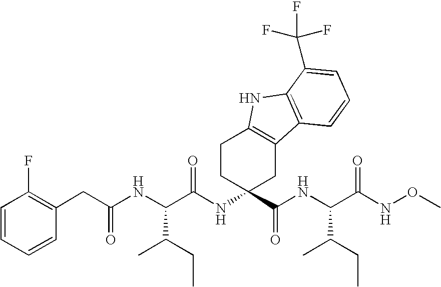

### Compound 39 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(morpholine-4-carbonyl)-butyl]-amide

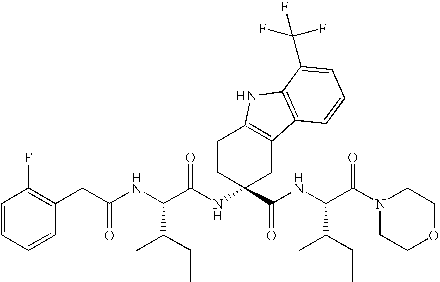

### Compound 40 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-ethylcarbamoyl-2-methylbutyl)-amide

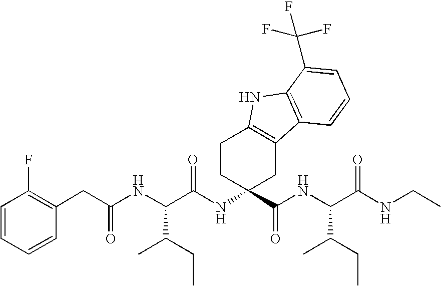

### Compound 41 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-ethoxycarbamoyl-2-methylbutyl)-amide

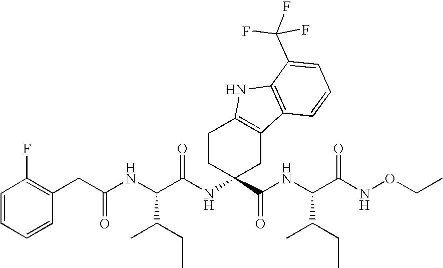

### Compound 42 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylcarbamoyl)-butyl]-amide

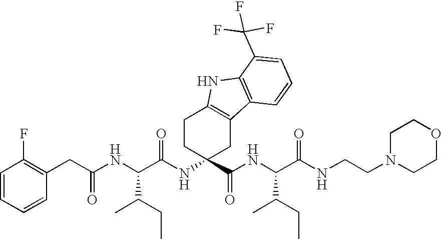

### Compound 43 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-butylcarbamoyl)-2-methyl-butyl]-amide

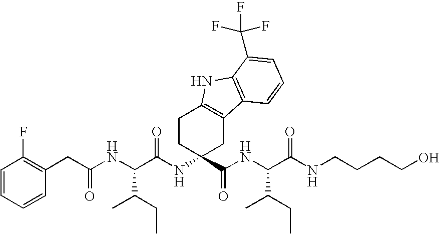

### Compound 44 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-morpholin-4-yl-propylcarbamoyl)-butyl]-amide

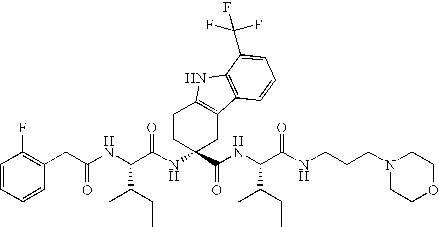

### Compound 45 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(1-methyl-piperidin-4-ylmethyl)-carbamoyl]-butyl}-amide

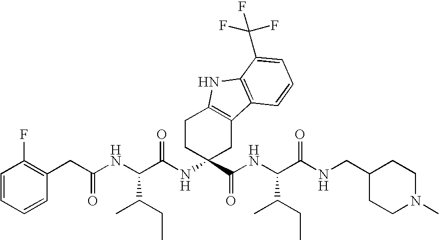

### Compound 46 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(tetrahydro-pyran-4-ylmethyl)-carbamoyl]-butyl}-amide

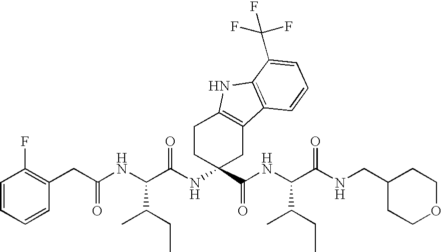

### Compound 47 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylthiocarbamoyl)-butyl]-amide

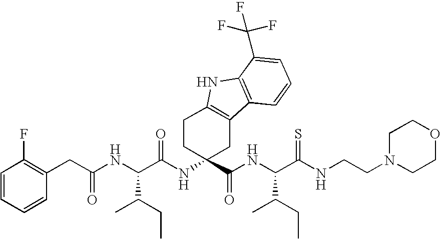

### Compound 48 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(1-formyl-piperidin-4-ylmethyl)-carbamoyl]-2-methyl-butyl}-amide

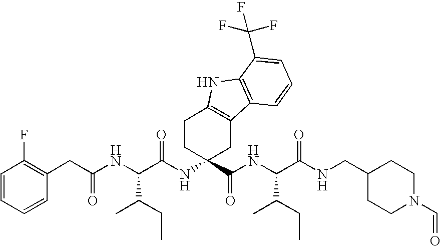

### Compound 49 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(1-acetyl-piperidin-4-ylmethyl)-carbamoyl]-2-methyl-butyl}-amide

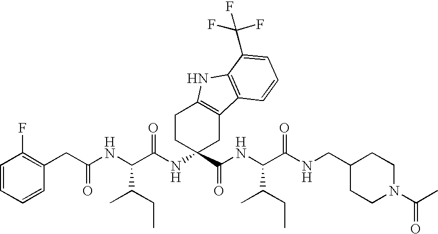

### Compound 50 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-4-ylmethyl)carbamoyl]-butyl}-amide

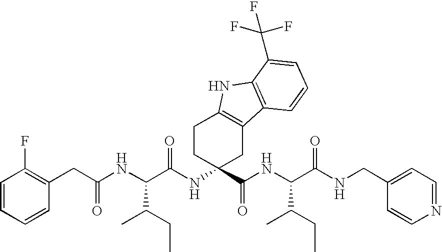

### Compound 51 (R)-3-{(S)-2-[2-(2-Fluoro-phenyl)-acetylamino)-3-methyl-pentanoylamino}-8-trifluoromethyl-2,3,4,9-tetrahydro-1H-carbazole-3-carboxylic acid f(S)-1-(2-diethylamino-ethylcarbamoyl)-2-methyl-butyl]-amide

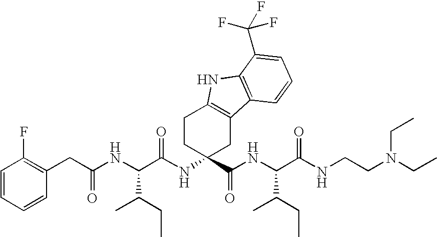

### Compound 52 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[K(tetrahydro-pyran-4-ylmethyl)-thiocarbamoyl]-butyl}-amide

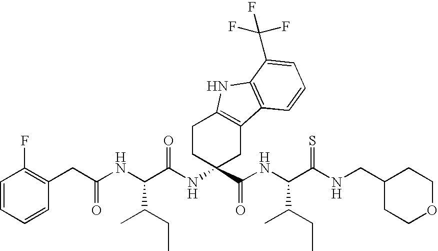

### Compound 53 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-butylthiocarbamoyl)-2-methyl-butyl]-amide

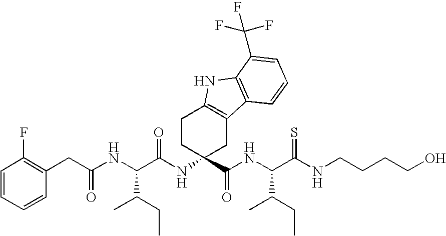

### Compound 54 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylthiocarbamoyl)-butyl]-amide

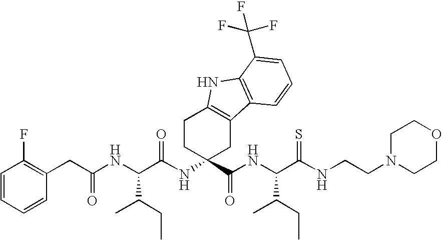

### Compound 55 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid [(S)-2-methyl-1-(2-morpholin-4-yl-ethylcarbamoyl)-butyl]-amide

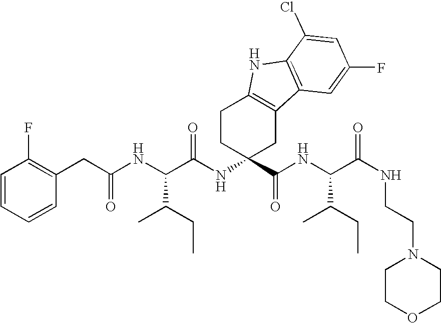

### Compound 56 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(piperidin-4-ylmethyl)-carbamoyl]-butyl}-amide

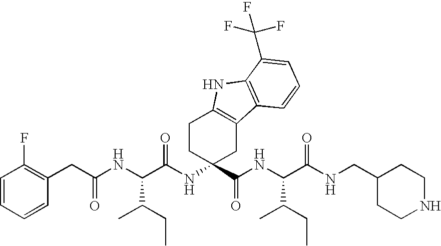

### Compound 57 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-hydroxy-ethylcarbamoyl)-2-methyl-butyl]-amide

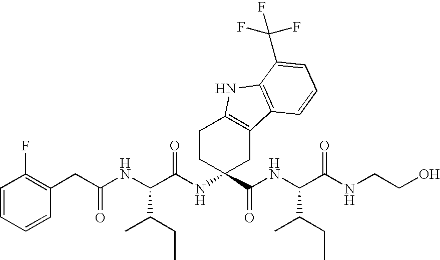

### Compound 58 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide

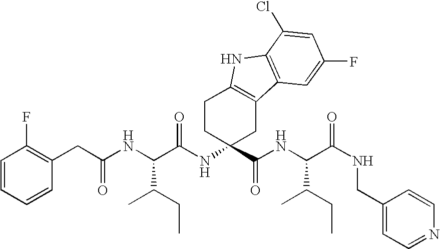

### Compound 59 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(5-hydroxy-pentylcarbamoyl)-2-methyl-butyl]-amide

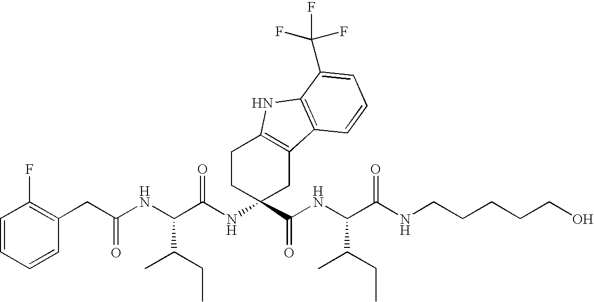

### Compound 60 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-4-ylmethyl)thiocarbamoyl]-butyl}-amide

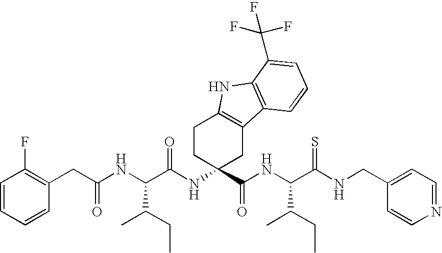

### Compound 61 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid [(S)-2-methyl-1-(2-morpholin-4-yl-ethylthiocarbamoyl)-butyl]-amide

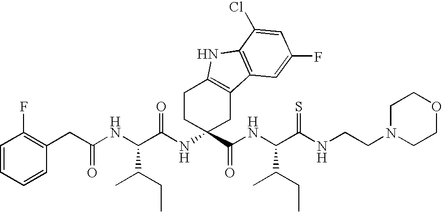

### Compound 62 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-methyl-1-{[(tetrahydro-pyran-4-ylmethyl)-amino]-methyl}-butyl)-amide

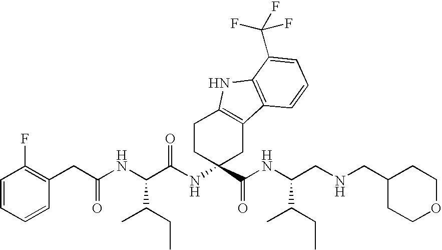

### Compound 63 (4-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-butyl)phosphonic acid diethyl ester

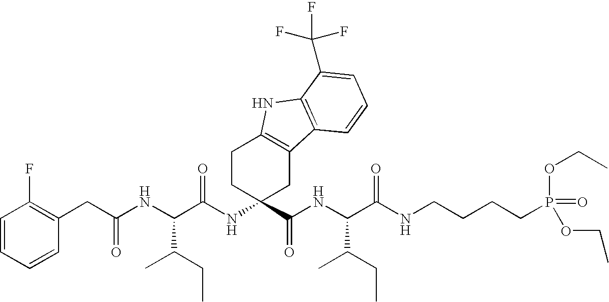

### Compound 64 (4-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-butyl)phosphonic acid

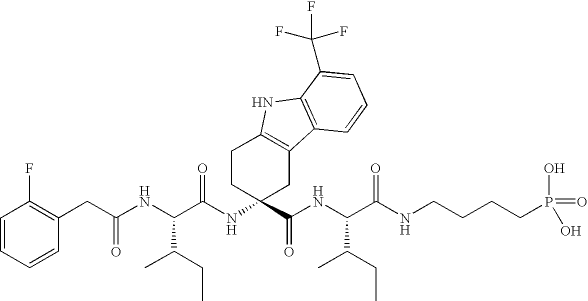

### Compound 66 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(2-morpholin-4-yl-ethylamino)-methyl]-butyl}-amide

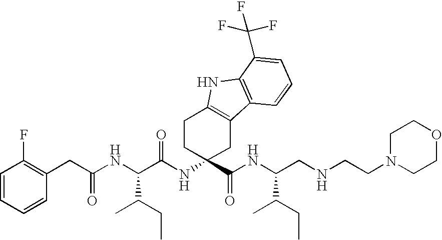

### Compound 67 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-phenylcarbamoyl)-2-methyl-butyl]-amide

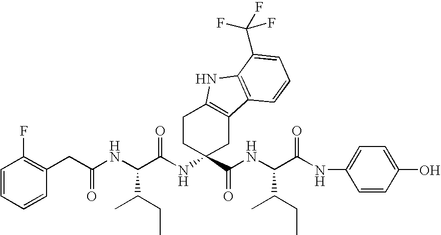

### Compound 68 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-methoxy-phenylcarbamoyl)-2-methyl-butyl]-amide

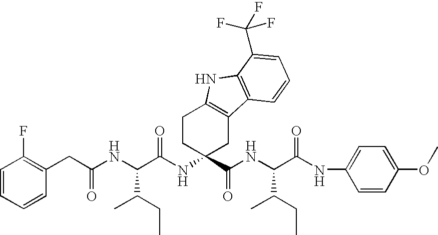

### Compound 69 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-phenylcarbamoyl)-2-methyl-butyl]-amide

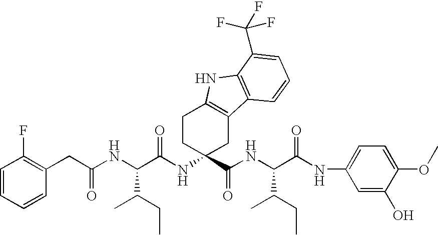

### Compound 70 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2,4-dihydroxy-phenylcarbamoyl)-2-methyl-butyl]-amide

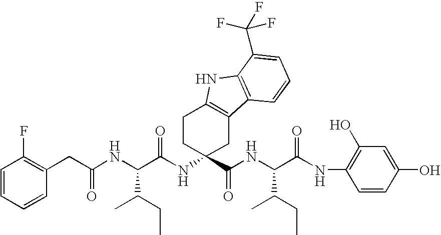

### Compound 71 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-hydroxy-4-methoxy-phenylcarbamoyl)-2-methyl-butyl]-amide

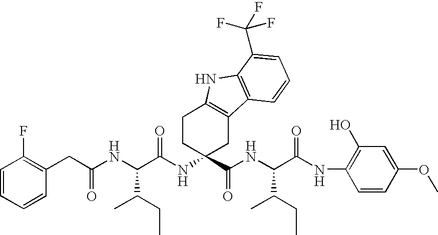

### Compound 72 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2,4,6-trimethoxy-phenylcarbamoyl)-butyl]-amide

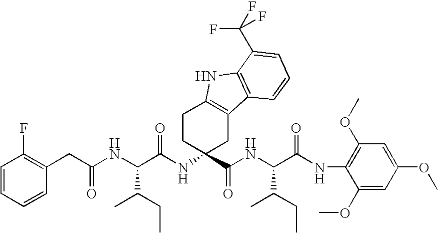

### Compound 73 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-cyclohexylcarbamoyl)-2-methyl-butyl]-amide

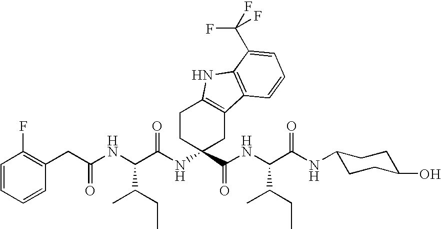

### Compound 74 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-imidazol-1-yl-propylthiocarbamoyl)-2-methyl-butyl]-amide

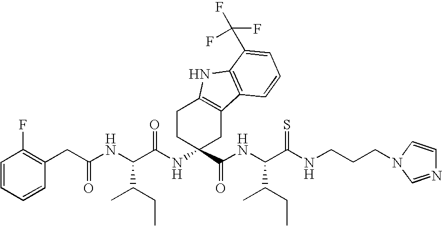

### Compound 75 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(R)-1-(3-imidazol-1-yl-propylthiocarbamoyl)-2-methyl-butyl]-amide

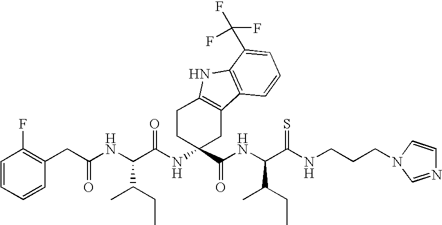

### Compound 76 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-thiophen-2-yl-ethylthiocarbamoyl)-butyl]-amide

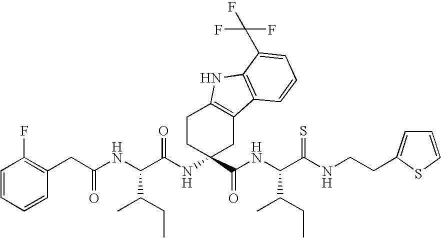

### Compound 77 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-3-ylmethyl)thiocarbamoyl]-butyl}-amide

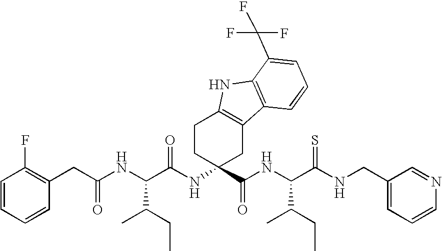

### Compound 78 ((S)-1-{(R)-6,8-Dichloro-3-[(S)-3-methyl-1-(1H-tetrazol-5-yl)butylcarbamoyl]-2,3,4,9-tetrahydro-1H-carbazol-3-ylcarbamoyl}-2-methyl-butyl)-carbamic acid benzyl ester

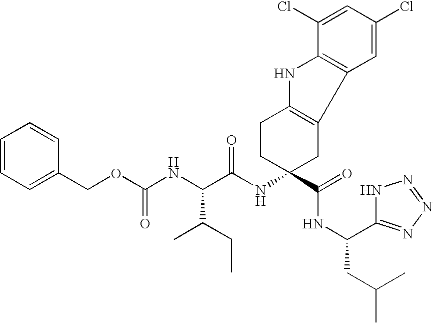

### Compound 80 5-{(S)-1-[(3-{2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester

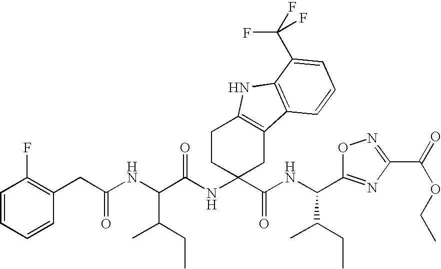

### Compound 81 5-{(S)-1-[((R)-3-{(R)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester

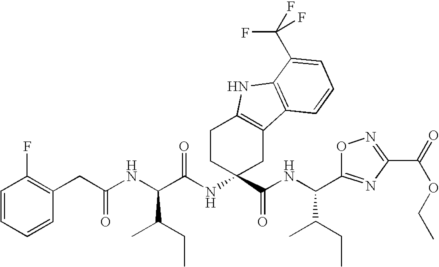

### Compound 82 (R)-3-{(R)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide

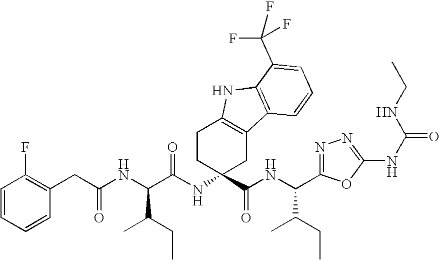

### Compound 83 (S)-3-{(R)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide

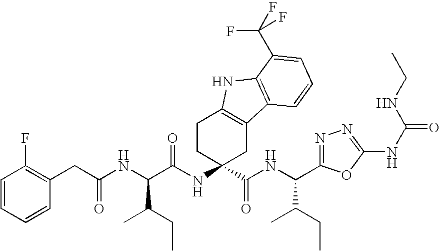

### Compound 85 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(2-diethylamino-ethylcarbamoyl)methyl]-amide

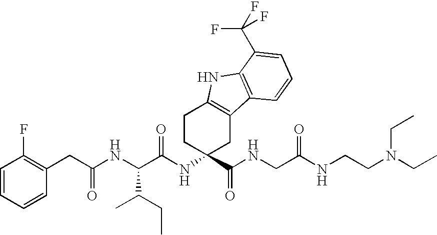

### Compound 86 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(2-morpholin-4-yl-ethylcarbamoyl)methyl]-amide

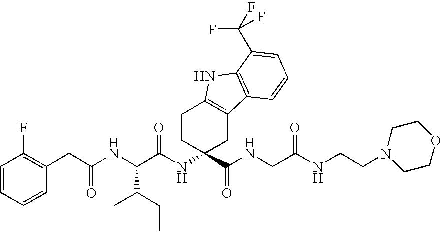

### Compound 87 (S)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide

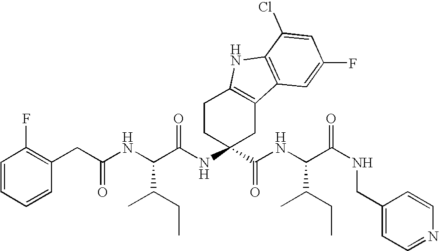

### Compound 88 (R)-8-Chloro-6-fluoro-3-{(S)-2-[2-(2-fluoro-phenyl)thioacetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide

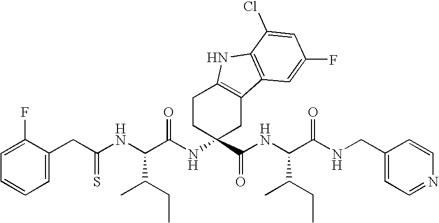

### Compound 89 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyridin-4-yl-ethylthiocarbamoyl)-butyl]-amide

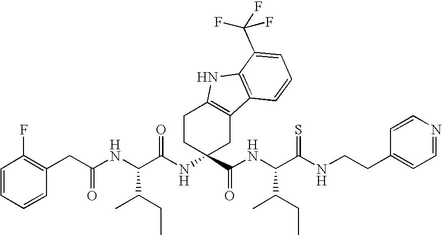

### Compound 90 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-cyclohexylthiocarbamoyl)-2-methyl-butyl]-amide

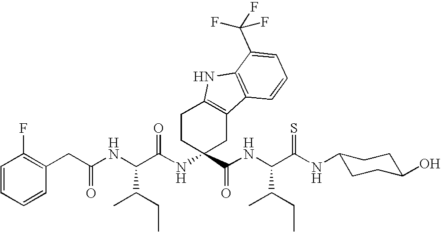

### Compound 91 2-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-5-methoxy-benzoic acid

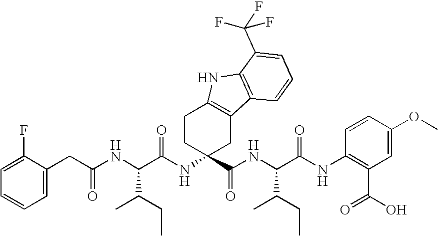

### Compound 92 Phosphoric acid diethyl ester 5-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-2-methoxy-phenyl ester

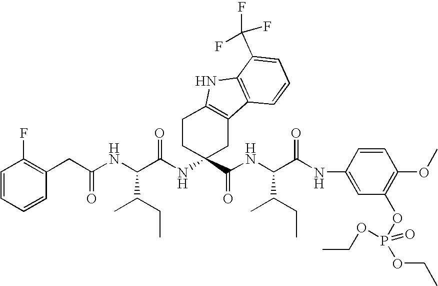

### Compound 93 Dimethylamino-acetic acid 4-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-butyl ester

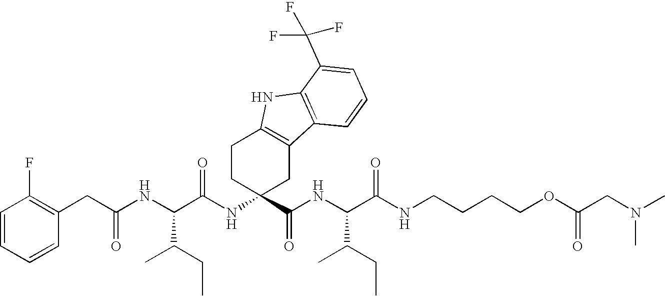

### Compound 94 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-benzylcarbamoyl)-2-methyl-butyl]-amide

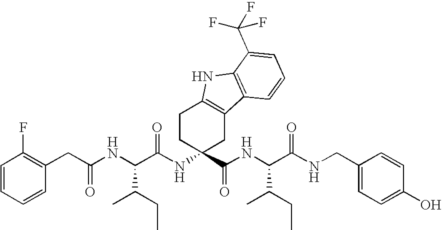

### Compound 95 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-benzylcarbamoyl)-2-methyl-butyl]-amide

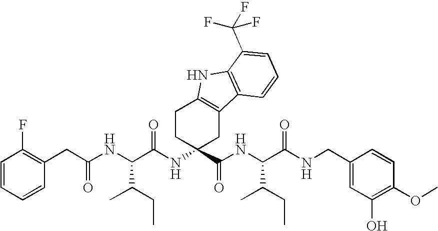

### Compound 96 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-3-methoxy-benzylcarbamoyl)-2-methyl-butyl]-amide

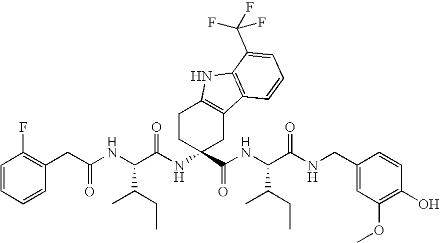

### Compound 97 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-methoxyphenyl-thiocarbamoyl)-2-methyl-butyl]-amide

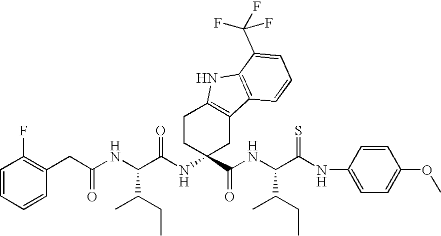

### Compound 98 5-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-2-methoxy-benzoic acid

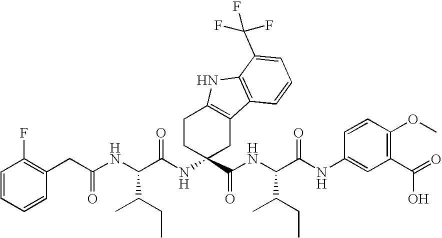

### Compound 99 5-{(S)-2-[((R)-3-{(S)-2-[2-(2,-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanethioylamino}-2-methoxy-benzoic acid

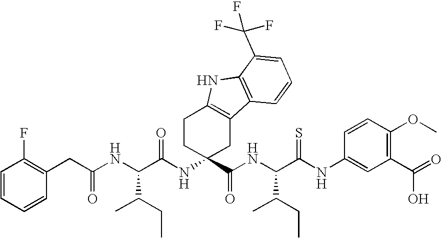

### Compound 100 Carbonic acid 4-{(S)-2-[((R)-3-{(S)-2-[2-(2-fluoro-phenyl)acetylamino]-3-methyl-pentanoylamino}-8-trifluoromethyl-2,3,4,9-tetrahydro-1H-carbazole-3-carbonyl)-amino]-3-methyl-pentanoylamino}-butyl ester 2-[2-(2-methoxy-ethoxy)-ethoxy]-ethyl ester

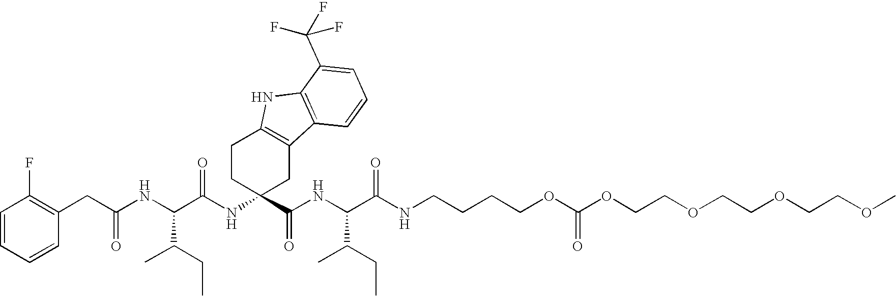

### Compound 101 Phosphoric acid mono-(4-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-butyl) ester

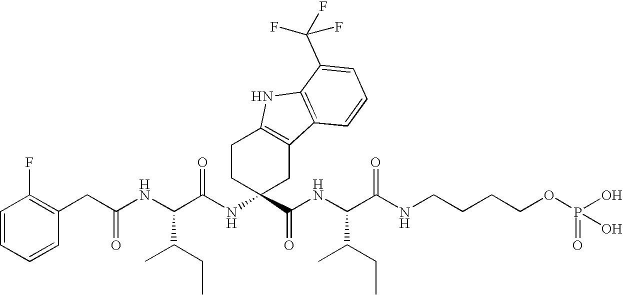

### Compound 102 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-propylcarbamoyl)-2-methyl-butyl]-amide

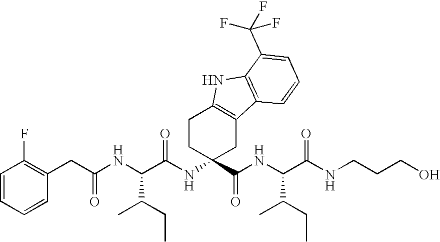

### Compound 103 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-benzylthiocarbamoyl-2-methyl-butyl)-amide

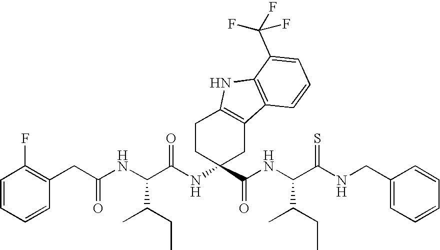

### Compound 104 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(2-methoxy-pyridin-4-ylmethyl)-thiocarbamoyl]-2-methyl-butyl}-amide

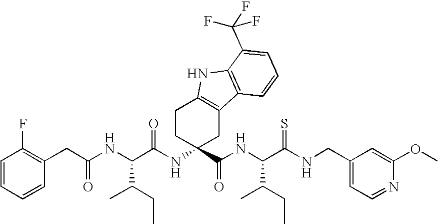

### Compound 105 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrimidin-5-ylmethyl)-thiocarbamoyl]-butyl}-amide

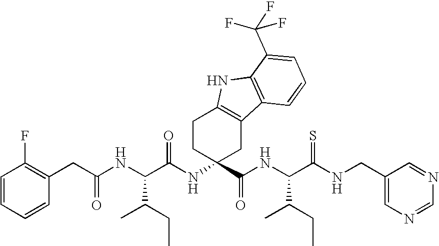

### Compound 106 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrazin-2-ylmethyl)-thiocarbamoyl]-butyl}-amide

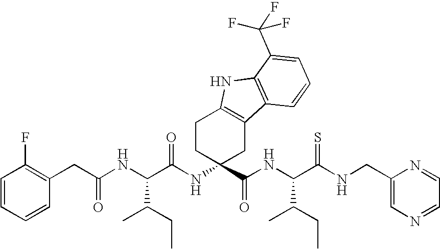

### Compound 107 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(6-chloro-pyridin-3-ylmethyl)thiocarbamoyl]-2-methyl-butyl}-amide

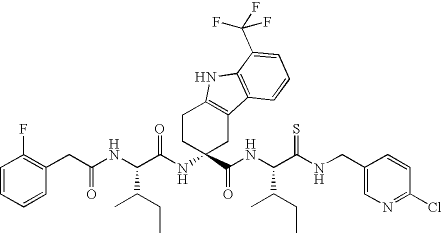

### Compound 108 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyridin-3-yl-ethylthiocarbamoyl)-butyl]-amide

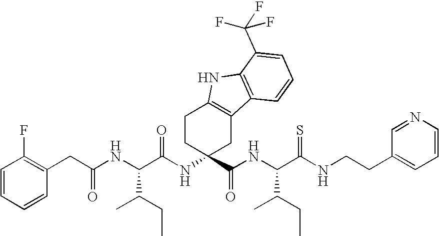

### Compound 109 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrimidin-4-ylmethyl)-thiocarbamoyl]-butyl}-amide

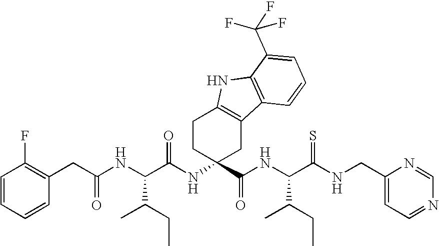

### Compound 110 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-imidazol-1-yl-ethylthiocarbamoyl)-2-methyl-butyl]-amide

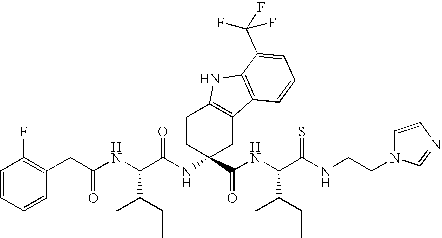

### Compound 111 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyrazol-1-yl-ethylthiocarbamoyl)-butyl]-amide

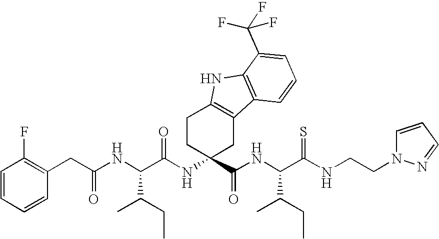

### Compound 112 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-[1,2,4]triazol-1-yl-ethylthiocarbamoyl)-butyl]-amide

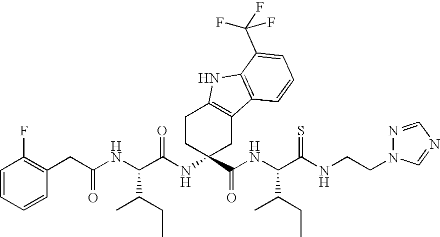

### Compound 113 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-[1,2,4]triazol-1-yl-propylthiocarbamoyl)-butyl]-amide

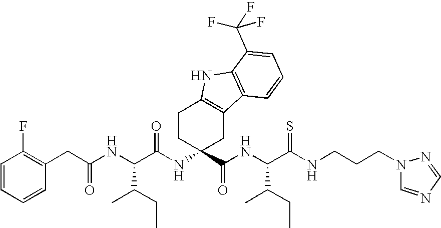

### Compound 114 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(4-[1,2,4]triazol-1-yl-butylthiocarbamoyl)-butyl]-amide

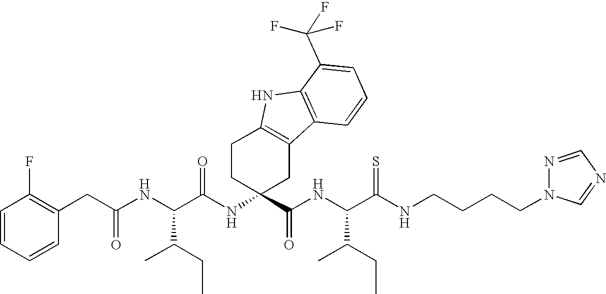

### Compound 115 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(furan-2-ylmethyl)thiocarbamoyl]-2-methyl-butyl}-amide

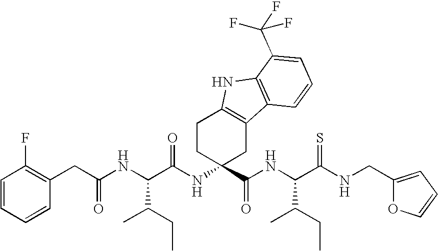

### Compound 116 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(furan-3-ylmethyl)thiocarbamoyl]-2-methyl-butyl}-amide

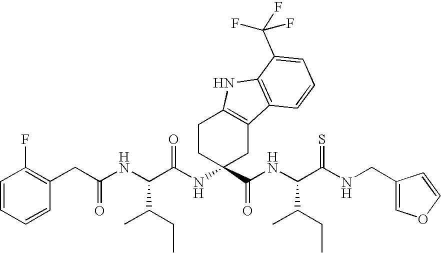

### Compound 117 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-furan-2-yl-ethylthiocarbamoyl)-2-methyl-butyl]-amide

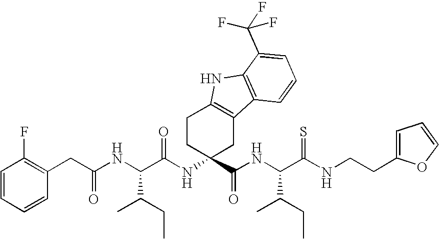

### Compound 118 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(5-methyl-[1,3,4]oxadiazol-2-ylmethyl)-thiocarbamoyl]-butyl}-amide

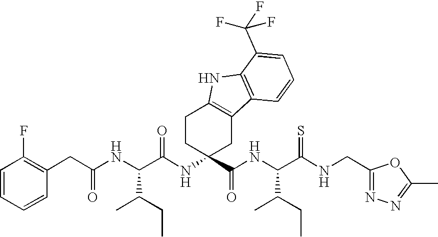

### Compound 119 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[2-(tetrahydro-pyran-4-yl)-ethylthiocarbamoyl]-butyl}-amide

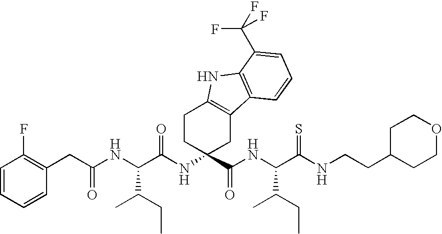

### Compound 120 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(tetrahydro-pyran-4-yl-thiocarbamoyl)-butyl]-amide

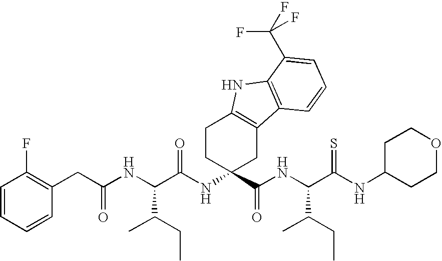

### Compound 121 5-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-2-hydroxy-benzoic acid

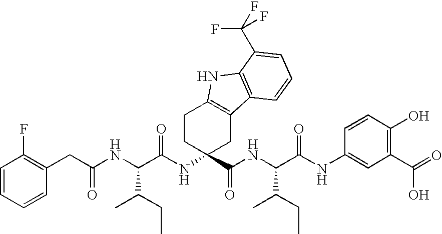

### Compound 122 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-fluoro-4-hydroxy-phenylcarbamoyl)-2-methyl-butyl]-amide

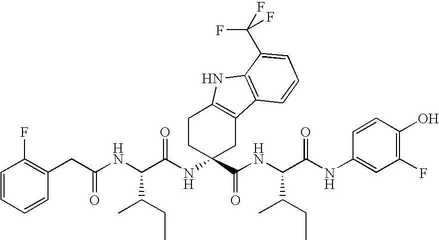

### Compound 123 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-benzylthiocarbamoyl)-2-methyl-butyl]-amide

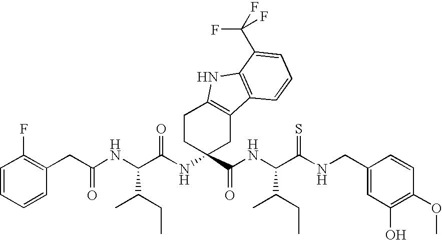

### Compound 124 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-hydrazinocarbonyl-2-methylbutyl)-amide

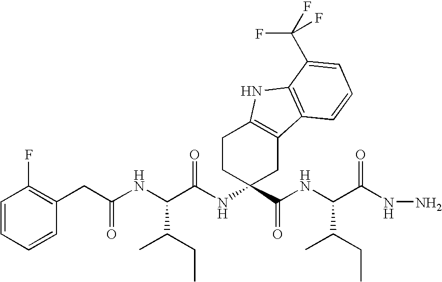

### Compound 125 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-phenylthiocarbamoyl)-2-methyl-butyl]-amide

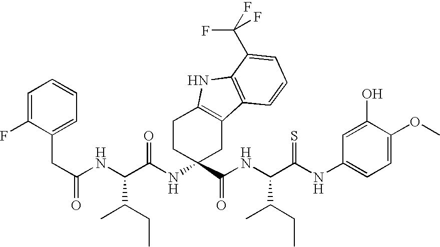

### Compound 126 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-oxo-pyrrolidin-3-yl)-amide

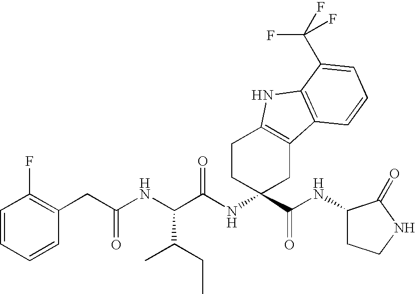

### Compound 127 (S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-imidazol-1-yl-propyl ester

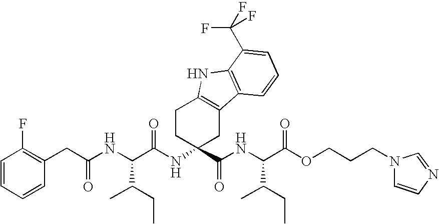

### Compound 128 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-imidazol-1-yl-propyl ester

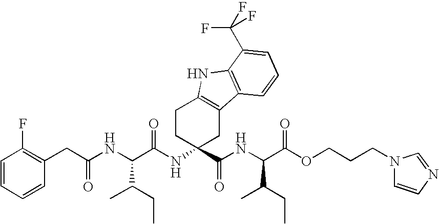

### Compound 129 ((S)-1-{(R)-6,8-Dichloro-3-[(S)-1-(4-hydroxy-benzylcarbamoyl)-2-methyl-butylcarbamoyl]-2,3,4,9-tetrahydro-1H-carbazol-3-ylcarbamoyl}-2-methyl-butyl)-carbamic acid benzyl ester

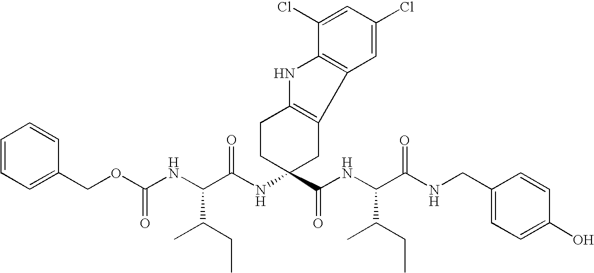

### Compound 130 ((S)-1-{(S)-6,8-Dichloro-3-[(S)-1-(4-hydroxy-benzylcarbamoyl)-2-methyl-butylcarbamoyl]-2,3,4,9-tetrahydro-1H-carbazol-3-ylcarbamoyl}-2-methyl-butyl)-carbamic acid benzyl ester

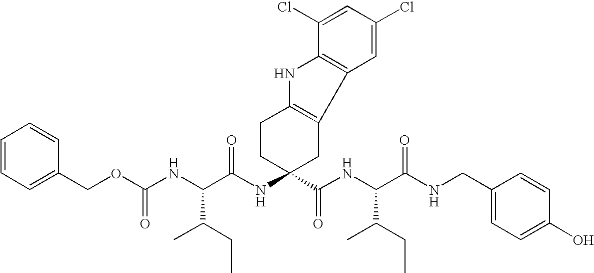

### Compound 131 (S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid pyridin-4-ylmethyl ester

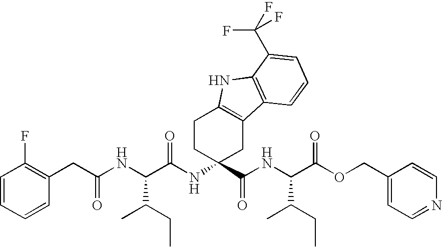

### Compound 132 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid pyridin-4-ylmethyl ester

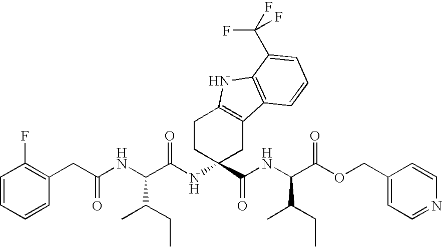

### Compound 133 2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 2-dimethylamino-ethyl ester

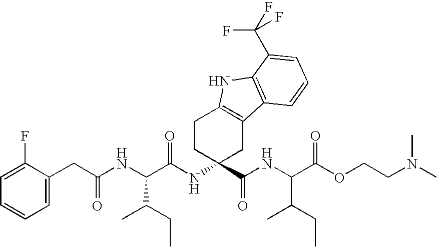

### Compound 134 (S)-2-t((R)-3-{(S)-2-[2-(2-Fluoro-phenyl)-acetylamino)-3-methyl-pentanoic(amino}-8-trifluoromethyl-2,3,4,9-tetrahydro-1H-carbazole-3-carbonyl)-amino]-3-methyl-pentanoic acid 3-hydroxy-4-methoxy-benzyl ester

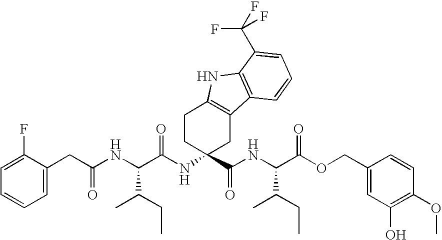

### Compound 135 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-hydroxy-4-methoxy-benzyl ester

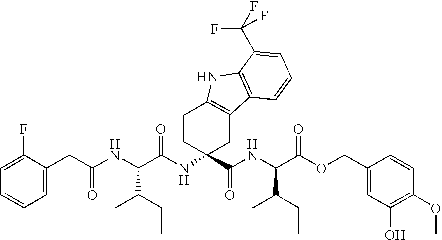

### Compound 136 [(S)-1-((S)-6,8-Dichloro-3-{(S)-2-methyl-1-[(tetrahydro-pyran-4-ylmethyl)-carbamoyl]-butylcarbamoyl}-2,3,4,9-tetrahydro-1H-carbazol-3-ylcarbamoyl)-2-methyl-butyl]-carbamic acid benzyl ester

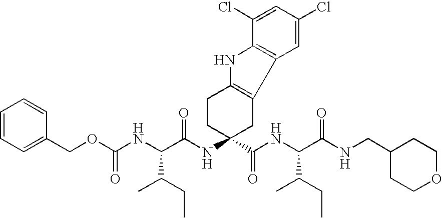

### Compound 137 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(quinolin-6-ylcarbamoyl)-butyl]-amide

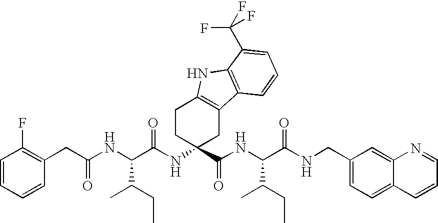

### Compound 138 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(R)-2-methyl-1-(quinolin-6-ylcarbamoyl)-butyl]-amide

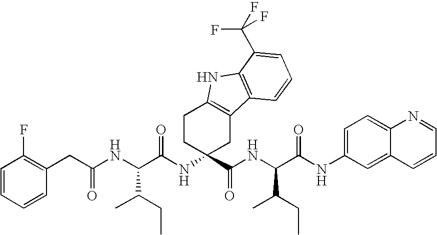

### Compound 139 [(S)-1-((R)-6,8-Dichloro-3-{(S)-2-methyl-1-[(tetrahydro-pyran-4-ylmethyl)-thiocarbamoyl]-butylcarbamoyl}-2,3,4,9-tetrahydro-1H-carbazol-3-ylcarbamoyl)-2-methyl-butyl]-carbamic acid benzyl ester

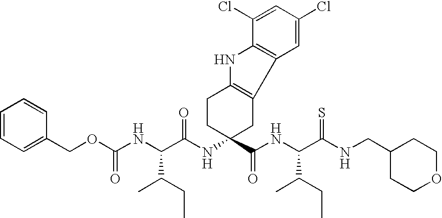

### Compound 140 ((S)-1-{(R)-6,8-Dichloro-3-[(S)-1-(4-hydroxy-3-methoxy-phenylcarbamoyl)-2-methyl-butylcarbamoyl]-2,3,4,9-tetrahydro-1H-carbazol-3-ylcarbamoyl}-2-methyl-butyl)-carbamic acid benzyl ester

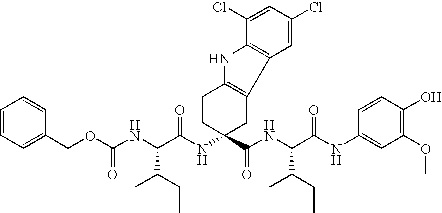

### Compound 141 ((S)-1-{(S)-6,8-Dichloro-3-[(S)-1-(4-hydroxy-3-methoxy-phenylcarbamoyl)-2-methyl-butylcarbamoyl]-2,3,4,9-tetrahydro-1H-carbazol-3-ylcarbamoyl}-2-methyl-butyl)-carbamic acid benzyl ester

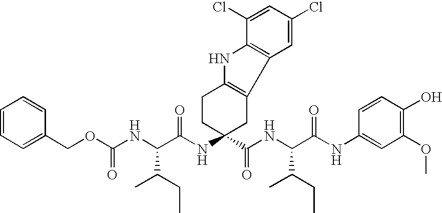

### Compound 142 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-oxo-piperidin-3-yl)-amide

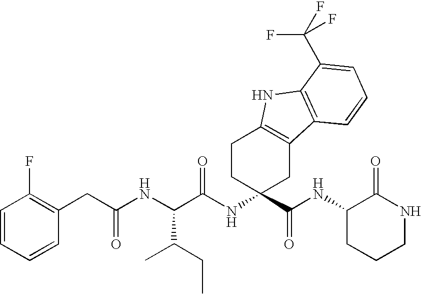

### Compound 143 [(S)-1-((R)-6,8-Dichloro-3-{(S)-2-methyl-1-[(tetrahydro-pyran-4-ylmethyl)-carbamoyl]-butylcarbamoyl}-2,3,4,9-tetrahydro-1H-carbazol-3-ylcarbamoyl)-2-methyl-butyl]-carbamic acid benzyl ester

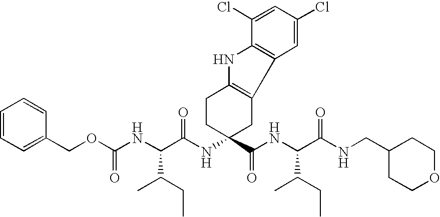

### Compound 144 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(N′-phenyl-hydrazinocarbonyl)-butyl]-amide

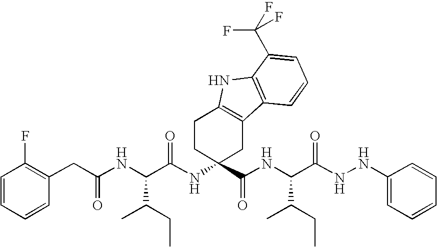

### Compound 145 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-3-methylsulfanyl-1-thiocarbamoyl-propyl)-amide

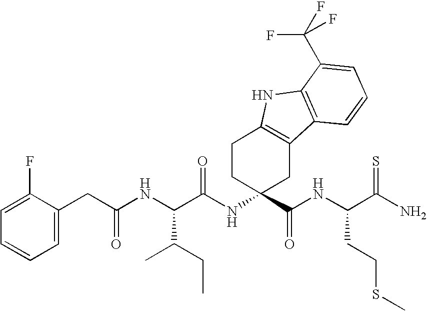

### Compound 146 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(quinolin-5-ylcarbamoyl)-butyl]-amide

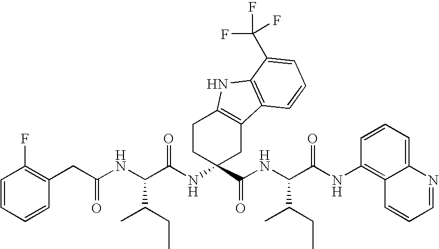

### Compound 147 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(isoquinolin-5-ylcarbamoyl)-2-methyl-butyl]-amide

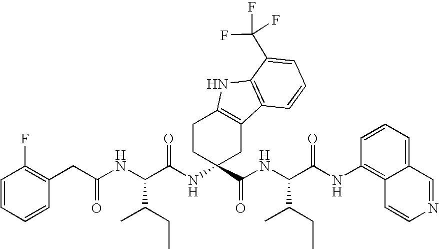

### Compound 148 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(2-tetrahydro-pyran-4-yl-acetylamino)-methyl]-butyl}-amide

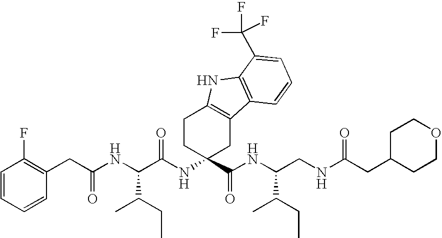

### Compound 149 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-methyl-1-{[(tetrahydro-pyran-4-carbonyl)-amino]-methyl}-butyl)-amide

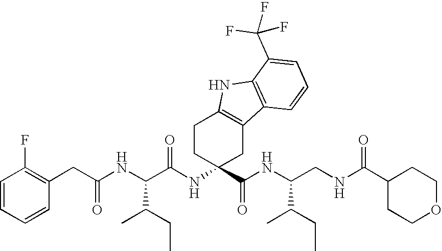

### Compound 150 (R)-3-((S)-2-[2-(2-Fluoro-phenyl)-acetylamino]-3-methyl-pentanoylamino)-8-trifluoromethyl-2,3,4,9-tetrahydro-1H-carbazole-3-carboxylic acid {(S)-2-methyl-1-[(3-morpholin-4-ylpropionylamino)-methyl]-butyl}-amide

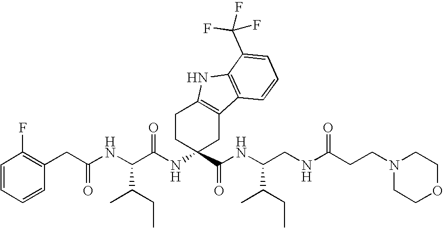

### Compound 151 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(3-imidazol-1-yl-propionylamino)-methyl]-2-methyl-butyl}-amide

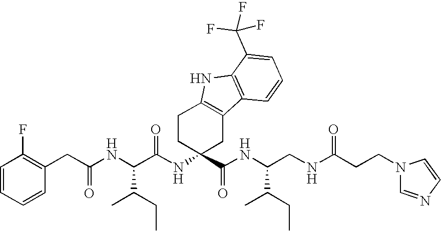

### Compound 152 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[3-(tetrahydro-pyran-4-ylmethyl)-ureidomethyl]-butyl}-amide

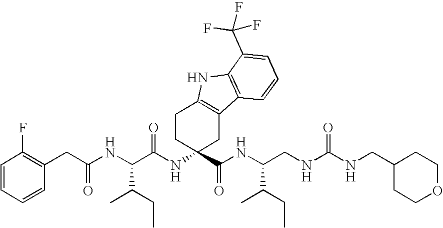

### Compound 153 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-tetrahydro-pyran-4-yl-acetylamino)-butyl]-amide

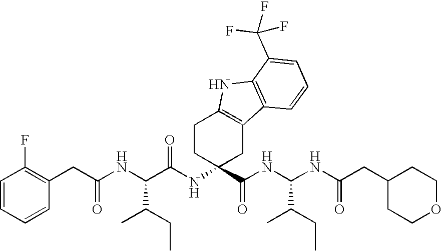

### Compound 154 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(tetrahydro-pyran-4-carbonyl)-amino]-butyl}-amide

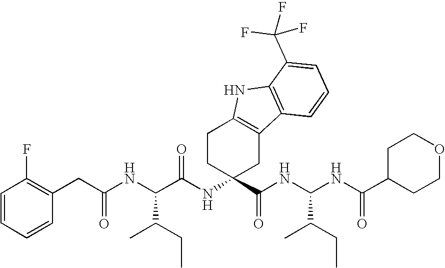

### Compound 155 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-morpholin-4-yl-propionylamino)-butyl]-amide

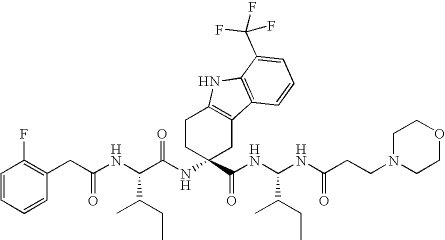

### Compound 156 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-imidazol-1-yl-propionylamino)-2-methyl-butyl]-amide

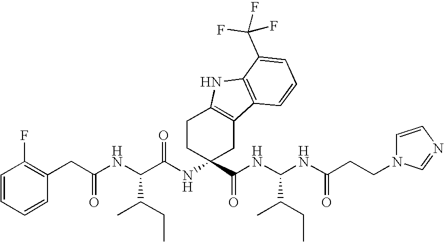

### Compound 157 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(R)-2-methyl-1-[3-(tetrahydro-pyran-4-ylmethyl)-ureido]-butyl}-amide

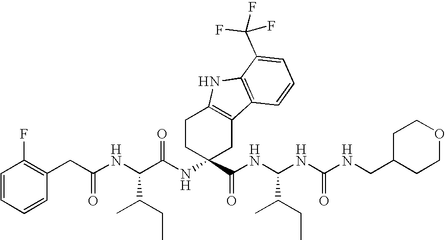

### Compound 158 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((R)-2-methyl-1-{[(tetrahydro-pyran-4-ylmethyl)-carbamoyl]-methyl}-butyl)-amide

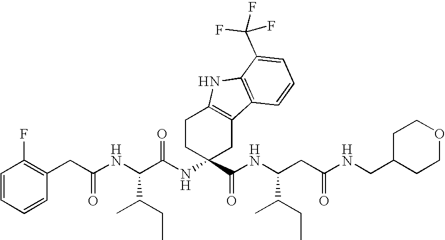

### Compound 159 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-phenyl)hydrazinocarbonyl]-2-methyl-butyl}-amide

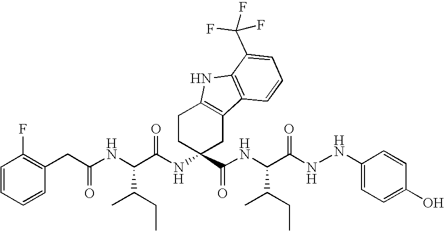

### Compound 160 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-methoxy-phenyl)hydrazinocarbonyl]-2-methyl-butyl}-amide

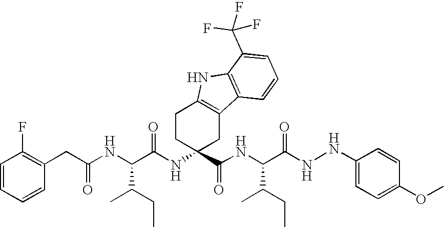

### Compound 161 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(3-hydroxy-4-methoxybenzyl)-hydrazinocarbonyl]-2-methyl-butyl}-amide

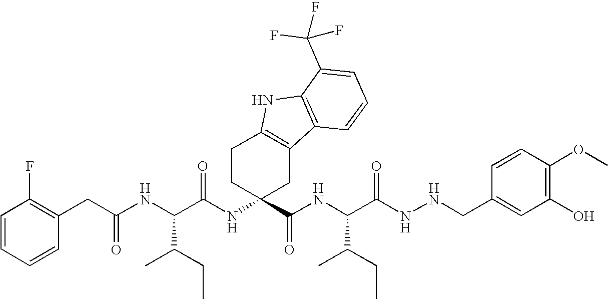

### Compound 162 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-3-methoxybenzyl)-hydrazinocarbonyl]-2-methyl-butyl}-amide

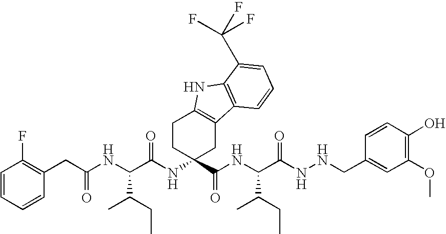

### Compound 163 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(N′-acetyl-hydrazinocarbonyl)-2-methyl-butyl]-amide

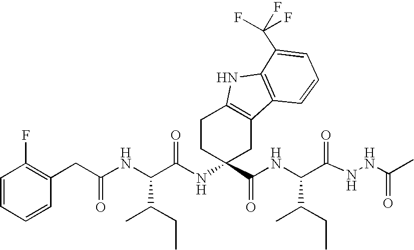

### Compound 164

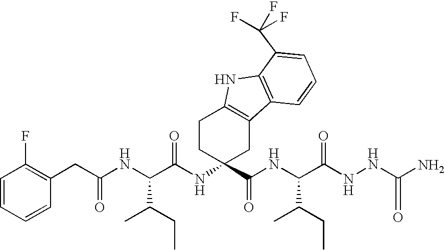

### Compound 165 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-benzoyl)hydrazinocarbonyl]-2-methyl-butyl}-amide

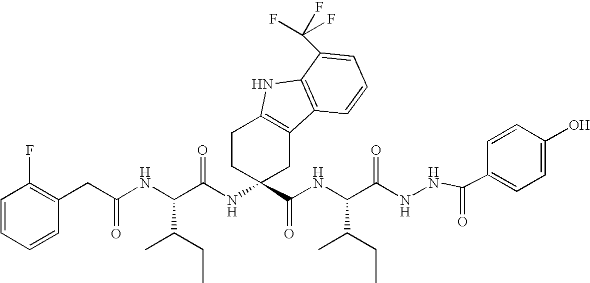

For the avoidance of doubt, if chemical name and chemical structure of the above illustrated compounds do not correspond by mistake, the chemical structure is regarded to unambiguously define the compound.

The terms indicated for explanation of the above compounds of the invention always, unless indicated otherwise in the description or in the claims, have the following meanings:

The term “unsubstituted” means that the corresponding radical, group or moiety has no substituents.

The term “substituted” means that the corresponding radical, group or moiety has one or more substituents. Where a radical has a plurality of substituents, and a selection of various substituents is specified, the substituents are selected independently of one another and do not need to be identical.

The term “alkyl” for the purposes of this invention refers to acyclic saturated or unsaturated hydrocarbon radicals which may be branched or straight-chain and have 1 to 8 carbon atoms, i.e. C₁₋₈-alkanyls, C₂₋₈-alkenyls and C₂₋₈-alkynyls. Alkenyls have at least one C—C double bond and alkynyls at least one C—C triple bond. Alkynyls may additionally also have at least one C—C double bond. Examples of suitable alkyl radicals are methyl, ethyl, n-propyl, isopropyl, n-butyl, isobutyl, sec-butyl, tert-butyl, n-pentyl, iso-pentyl, neo-pentyl, tert-pentyl, 2- or 3-methyl-pentyl, n-hexyl, 2-hexyl, isohexyl, n-heptyl, n-octyl, n-nonyl, n-decyl, n-undecyl, n-dodecyl, n-tetradecyl, n-hexadecyl, n-octadecyl, n-icosanyl, n-docosanyl, ethylenyl (vinyl), propenyl (—CH₂CH═CH₂; —CH═CH—CH₃, —C(═CH₂)—CH₃), butenyl, pentenyl, hexenyl, heptenyl, octenyl, octadienyl, octadecenyl, octadec-9-enyl, icosenyl, icos-11-enyl, (Z)-icos-11-enyl, docosnyl, docos-13-enyl, (Z)-docos-13-enyl, ethynyl, propynyl (—CH₂—C≡CH, —C≡C—CH₃), butynyl, pentynyl, hexynyl, heptynyl, octynyl,

The term “(C₉-C₃₀)alkyl” for the purposes of this invention refers to acyclic saturated or unsaturated hydrocarbon radicals which may be branched or straight-chain and have 9 to 30 carbon atoms, i.e. C₉₋₃₀-alkanyls, C₉₋₃₀-alkenyls and C₉₋₃₀-alkynyls. C₉₋₃₀-Alkenyls have at least one C—C double bond and C₉₋₃₀-alkynyls at least one C—C triple bond. C₉₋₃₀-Alkynyls may additionally also have at least one C—C double bond. Examples of suitable (C₉-C₃₀)alkyl radicals are tetradecyl, hexadecyl, octadecyl, eicosanyl, cis-13-docosenyl (erucyl), trans-13-docosenyl (brassidyl), cis-15-tetracosenyl (nervonyl) and trans-15-tetracosenyl.

The term “cycloalkyl” for the purposes of this invention refers to saturated and partially unsaturated non-aromatic cyclic hydrocarbon groups/radicals, having 1 to 3 rings, that contain 3 to 20, preferably 3 to 12, most preferably 3 to 8 carbon atoms. The cycloalkyl radical may also be part of a bi- or polycyclic system, where, for example, the cycloalkyl radical is fused to an aryl, heteroaryl or heterocyclyl radical as defined herein by any possible and desired ring member(s). The bonding to the compounds of the general formula (I) or (II) can be effected via any possible ring member of the cycloalkyl radical. Examples of suitable cycloalkyl radicals are cyclopropyl, cyclobutyl, cyclopentyl, cyclohexyl, cycloheptyl, cyclooctyl, cyclodecyl, cyclohexenyl, cyclopentenyl and cyclooctadienyl.

The term “heterocyclyl” for the purposes of this invention refers to a mono- or polycyclic system of 3 to 20, preferably 5 or 6 to 14 ring atoms comprising carbon atoms and 1, 2, 3, 4, or 5 heteroatoms, in particular nitrogen, oxygen and/or sulfur which are identical or different. The cyclic system may be saturated, mono- or polyunsaturated but may not be aromatic. In the case of a cyclic system consisting of at least two rings the rings may be fused or spiro- or otherwise connected. Such “heterocyclyl” radicals can be linked via any ring member. The term “heterocyclyl” also includes systems in which the heterocycle is part of a bi- or polycyclic saturated, partially unsaturated and/or aromatic system, such as where the heterocycle is fused to an “aryl”, “cycloalkyl”, “heteroaryl” or “heterocyclyl” group as defined herein via any desired and possible ring member of the heterocycyl radical. The bonding to the compounds of the general formula (I) or (II) can be effected via any possible ring member of the heterocycyl radical. Examples of suitable “heterocyclyl” radicals are pyrrolidinyl, thiapyrrolidinyl, piperidinyl, piperazinyl, oxapiperazinyl, oxapiperidinyl, oxadiazolyl, tetrahydrofuryl, imidazolidinyl, thiazolidinyl, tetrahydropyranyl, morpholinyl, tetrahydrothiophenyl, dihydropyranyl.

The term “aryl” for the purposes of this invention refers to aromatic hydrocarbon systems having 3 to 14, preferably 5 to 14, more preferably 6 to 14 carbon atoms. The term “aryl” also includes systems in which the aromatic cycle is part of a bi- or polycyclic saturated, partially unsaturated and/or aromatic system, such as where the aromatic cycle is fused to an “aryl”, “cycloalkyl”, “heteroaryl” or “heterocyclyl” group as defined herein via any desired and possible ring member of the aryl radical. The bonding to the compounds of the general formula (I) or (II) can be effected via any possible ring member of the aryl radical. Examples of suitable “aryl” radicals are phenyl, biphenyl, naphthyl and anthracenyl, but likewise indanyl, indenyl, or 1,2,3,4-tetrahydronaphthyl.

The term “heteroaryl” for the purposes of this invention refers to a 3 to 14, preferably 5 to 14, more preferably 5-, 6- or 7-membered cyclic aromatic hydrocarbon radical which comprises at least 1, where appropriate also 2, 3, 4 or 5 heteroatoms, preferably nitrogen, oxygen and/or sulfur, where the heteroatoms are identical or different. The number of nitrogen atoms is preferably 0, 1, 2, or 3, and that of the oxygen and sulfur atoms is independently 0 or 1. The term “heteroaryl” also includes systems in which the aromatic cycle is part of a bi- or polycyclic saturated, partially unsaturated and/or aromatic system, such as where the aromatic cycle is fused to an “aryl”, “cycloalkyl”, “heteroaryl” or “heterocyclyl” group as defined herein via any desired and possible ring member of the heteroaryl radical. The bonding to the compounds of the general formula (I) or (II) can be effected via any possible ring member of the heteroaryl radical. Examples of suitable “heteroaryl” are pyrrolyl, thienyl, furyl, imidazolyl, thiazolyl, isothiazolyl, oxazolyl, oxadiazolyl, isoxazolyl, pyrazolyl, pyridinyl, pyrimidinyl, pyridazinyl, pyrazinyl, indolyl, quinolinyl, isoquinolinyl, imidazolyl, triazolyl, triazinyl, tetrazolyl, phthalazinyl, indazolyl, indolizinyl, quinoxalinyl, quinazolinyl, pteridinyl, carbazolyl, phenazinyl, phenoxazinyl, phenothiazinyl, acridinyl.

For the purposes of the present invention, the terms “alkyl-cycloalkyl”, “cycloalkylalkyl”, “alkyl-heterocyclyl”, “heterocyclylalkyl”, “alkyl-aryl”, “arylalkyl”, “alkyl-heteroaryl” and “heteroarylalkyl” mean that alkyl, cycloalkyl, heterocycle, aryl and heteroaryl are each as defined above, and the cycloalkyl, heterocyclyl, aryl and heteroaryl radical is bonded to the compounds of the general formula (I) or (II) via an alkyl radical, preferably C₁-C₈-alkyl radical, more preferably C₁-C₄-alkyl radical.

The term “halogen”, “halogen atom” or “halogen substituent” (Hal—) for the purposes of this invention refers to one, where appropriate, a plurality of fluorine (F, fluoro), bromine (Br, bromo), chlorine (Cl, chloro), or iodine (I, iodo) atoms. The designations “dihalogen”, “trihalogen” and “perhalogen” refer respectively to two, three and four substituents, where each substituent can be selected independently from the group consisting of fluorine, chlorine, bromine and iodine. “Halogen” preferably means a fluorine, chlorine or bromine atom.

In yet another preferred embodiment, at least one LHRH antagonist as defined herein is provided that can be used for the preparation of a medicament for the herein disclosed treatments, wherein the intermediate dose is a total monthly dose in the range of 10 mg to 150 mg LHRH antagonist, preferably a total monthly dose in the range of 10 mg to 120 mg LHRH antagonist, more preferably a total monthly dose in the range of 10 mg to 40 mg LHRH antagonists, more preferably a total monthly dose in the range of 40 mg to 150 mg LHRH antagonists, more preferably a total monthly dose in the range of 60 mg to 150 mg LHRH antagonists, more preferably a total monthly dose in the range of 60 mg to 120 mg LHRH antagonists and most preferably a total monthly dose of 10 mg, 20 mg, 30 mg, 40 mg, 60 mg, 90 mg, 120 mg, 130 mg or 150 mg LHRH antagonist.

In yet a further preferred embodiment, such total monthly dose is to be administered as one single monthly administration or is to be administered twice a month (preferably biweekly), three-times a month or four-times a month (preferably weekly).

If a total monthly dose is to be administered biweekly or weekly, for instance, the total monthly dose is the sum of each single administration, where the single administrations need not to be identical. That is a total monthly dose of 40 mg LHRH antagonist can, for instance, be administered in two biweekly administrations of 20 mg+20 mg or 30 mg+10 mg or in four weekly administrations of 10 mg+10 mg+10 mg+10 mg or 20 mg+5 mg+10 mg+5 mg. A total monthly dose of 90 mg LHRH antagonist can, for instance, be administered in two biweekly administrations of 60 mg+30 mg or 30 mg+60 mg or 45 mg+45 mg or three-times a month as 30 mg+30 mg+30 mg.

In a further preferred embodiment, at least one LHRH antagonist is provided that can be used for the preparation of a medicament for the herein disclosed treatments, wherein the intermediate dose is a single daily dose of 0.1 mg to 250 mg LHRH antagonist, a single daily dose of 1 mg to 60 mg LHRH antagonist, a single daily dose of 50 mg to 150 mg LHRH antagonist or a single daily dose of 50 mg, 75 mg or 150 mg LHRH antagonist, wherein the single daily dose is administered over one day, two days, three days, four days, five days, six days or more days, 1 week, 2 weeks, 3 weeks, 4 weeks, 5 weeks, 6 weeks or more weeks, 1 months, 2 months, 3 months, 4 months, 5 months, 6 months or more months, wherein the administration of each single dose can be followed by a treatment-free period of one day, two days, three days, four days, five days, six days or more days, 1 week, 2 weeks, 3 weeks, 4 weeks, 5 weeks, 6 weeks or more weeks, 1 months, 2 months, 3 months, 4 months, 5 months, 6 months or more months.

In a preferred embodiment, at least one LHRH antagonist as defined herein is provided that can be used for the preparation of a medicament for the herein disclosed treatments with the herein disclosed doses, wherein the at least one LHRH antagonist is to be administered over a treatment period of 1 or 2 months followed by a treatment-free period of 1, 2, 3, 4 or 5 months, preferably 2 months or 5 months (one treatment cycle).

The term “treatment cycle” in the course of the present invention is defined as a treatment period of 1 or 2 months followed by a treatment-free period of at least one and up to five months. That is the shortest treatment cycle consists of a one-month treatment period and a one-month treatment-free period, whereas the longest treatment cycle consists of a two-months treatment period and a five-months treatment-free period. Preferred are a treatment cycle with a one-month or two-months treatment period and a two-months treatment-free period and a treatment cycle with a one-month or two-months treatment period and a five-months treatment-free period.

In a further preferred embodiment, at least one LHRH antagonist as defined herein is provided that can be used for the preparation of a medicament for the herein disclosed treatments with the herein disclosed doses, wherein the at least one LHRH antagonist is to be administered in a dose of:
5 mg LHRH antagonist four-times a month (preferably weekly) or three-times a month or twice a month (preferably biweekly), or
10 mg LHRH antagonist four-times a month (preferably weekly) or three-times a month or twice a month (preferably biweekly), or
15 mg LHRH antagonist four-times a month (preferably weekly) or three-times a month or twice a month (preferably biweekly), or
30 mg LHRH antagonist four-times a month (preferably weekly) or three-times a month or twice a month (preferably biweekly), or
60 mg LHRH antagonist as one single administration followed by 30 mg LHRH antagonist as one single administration two weeks later, or
60 mg LHRH antagonist twice a month (preferably biweekly),

over a treatment period of 1 or 2 months followed by a treatment-free period of 1, 2, 3, 4 or 5 months, preferably 2 months or 5 months (one treatment cycle).

In a yet further preferred embodiment, at least one LHRH antagonist as defined herein is provided that can be used for the preparation of a medicament for the herein disclosed treatments with the herein disclosed doses, wherein the treatment cycle is repeated after the end of the treatment-free period of the preceding treatment cycle once, twice, three-times, four-times, five-times or continuously (chronic treatment) and wherein each respective succeeding treatment cycle can be identical or different to each respective preceding treatment cycle.

That is, for instance, a treatment cycle with a one-month or two-months treatment period and a two-months treatment-free period can be followed by a treatment cycle with a one-month or two-months treatment period and a five-months treatment-free period or vice versa. A chronic treatment could, for instance, consist of consecutive treatment cycles with a one-month or two-months treatment period and a two-months treatment-free period or of consecutive treatment cycles with a one-month of two-months treatment period and a five-months treatment-free period or of consecutive treatment cycles with alternating one-month or two-months treatment periods and two- or five-months treatment-free periods in all possible ways.

It is known in the prior art, that testosterone castration levels of castrates and boys before reaching puberty are in the range between 0.3 to 1.2 ng/mL (“Labor und Diagnose, Herausgegeben von Lothar Thomas, 5. Erweiterte Auflage 2000, page 44, 44.2.5 Referenzbereich”).

In the course of the present invention, the terms “hormone” and “hormonal” within for instance the terms “hormone castration”, “chemical (hormonal) castration” or “hormone withdrawal symptoms” refer to follicle stimulating hormone (FSH), luteinizing hormone (LH) and/or testosterone. Preferably, chemical (hormonal) castration is a testosterone castration and refers to a testosterone blood level of equal or below 1.2 ng/mL, preferably 0.5 ng/mL.

In a preferred embodiment, at least one LHRH antagonist as defined herein is provided that can be used for the preparation of a medicament for the herein disclosed treatments with the herein disclosed doses, wherein the chemical (hormonal) castration is a testosterone castration referring to a testosterone blood level of equal or below 1.2 ng/mL, preferably 0.5 ng/mL.

In a further aspect of the present invention, the at least one LHRH antagonist as defined herein is provided that can be used for the preparation of a medicament for the herein disclosed treatments with the herein disclosed doses, wherein such medicament comprises at least one additional pharmacologically active substance.

In a yet further aspect of the present invention, the at least one LHRH antagonist as defined herein is provided that can be used for the preparation of a medicament for the herein disclosed treatments with the herein disclosed doses, wherein the medicament is applied before and/or during and/or after treatment with at least one additional pharmacologically active substance.

The above mentioned at least one additional pharmacologically active substance includes: smooth muscle relaxants, bladder relaxants, bladder smooth muscle relaxants, anticholinergic agents, muscarinic acetylcholine receptor antagonists, tricyclic antidepressants, Calcium antagonist, Calcium agonist, Calcium channel blockers, Calcium channel activators, Potassium antagonists, Potassium agonists, Potassium channel blockers, Potassium channel activators, alpha-adrenergic receptor antagonists, alpha-blockers, alpha-adrenoreceptor antagonists, β3-adrenoreceptor agonists, vanilloids, vanilloid receptor antagonists, and/or botulinum toxins, and preferably, the at least one additional pharmacologically active substance is selected from these agents.

Examples of suitable anticholinergic agents and/or muscarinic acetylcholine receptor antagonists include oxybutynin (Benzeneacetic acid alpha-cyclohexyl-alpha-hydroxy-, 4-(diethylamino)-2-butynyl ester; synonyms: Ditropan; Ditropan XL; Oxytrol; Uromax, Chemical Abstract Services Registry Number: 5633-20-5), flavoxate (4H-1-Benzopyran-8-carboxylic acid 3-methyl-4-oxo-2-phenyl-2-(1-piperidinyl)ethyl ester, Chemical Abstract Services Registry Number: 15301-69-6), propantheline (N-methyl-N-(1-methylethyl)-N-[2-[(9H-xanthen-9-ylcarbonyl)oxy]ethyl]-2-propanaminium, Chemical Abstract Services Registry Number: 298-50-0), dicyclomine ([1,1′-Bicyclohexyl]-1-carboxylic acid 2-(diethylamino)ethyl ester, synonyms: Bentyl; Bentylol; Dicycloverin; Dicycloverine; Diocyl; Wyovin, Chemical Abstract Services Registry Number: 77-19-0), tolterodine (2-[(1R)-3-[bis(1-methylethyl)amino]-1-phenylpropyl]-4-methyl-phenol, synonyms: Kabi 2234; PNU 200583; Chemical Abstract Services Registry Number: 124937-51-5), darifenancin (1-[2-(2,3-dihydro-5-benzofuranyl)ethyl]-alpha, alpha-diphenyl-(3S)-3-pyrrolidineacetamide, synonyms: UK 88525, Chemical Abstract Services Registry Number: 133099-04-4), solifenancin ((1S)-(3R)-1-azabicyclo[2.2.2]oct-3-yl 3,4-dihydro-1-phenyl-2(1H)-isoquinolinecarboxylate (1:1) Butanedioic acid, synonyms: Vesicare; YM 905, Chemical Abstract Services Registry Number: 242478-38-2), trospium chloride (3-[(2-hydroxy-2,2-diphenylacetyl)oxy]-, chloride (1:1), (1-alpha,3-beta,5-alpha)- Spiro[8-azoniabicyclo[3.2.1]octane-8,1′-pyrrolidinium], synonyms: Keptan; Relaspium; Sanctura; Spasmex; Spasmo 3; Spasmo-lyt; Chemical Abstract Services Registry Number: 10405-02-4), fesoterodine (Propanoic acid 2-methyl-, 2-[(1R)-3-[bis(1-methylethyl)amino]-1-phenylpropyl]-4-(hydroxymethyl)phenyl ester, Chemical Abstract Services Registry Number: 286930-02-7), imidafenacin (2-methyl-alpha, alpha-diphenyl-1H-Imidazole-1-butanamide, synonyms: KRP 197; ONO 8025, Chemical Abstract Services Registry Number: 170105-16-5), PSD-506.

Examples of suitable tricyclic antidepressants include imipramine (10,11-dihydro-N,N-dimethyl-5H-Dibenz[b,f]azepine-5-propanamine, synonyms: Antideprin; Berkomine; Melipramine, NSC 169866; Org 2463; Prazepine, Chemical Abstract Services Registry Number: 50-49-7).

Examples of suitable calcium antagonists include terodiline (N-(1,1-dimethylethyl)alpha-methyl-gamma-phenyl-benzenepropanamine, Chemical Abstract Services Registry Number: 15793-40-5).

Examples of suitable alpha-adrenergic receptor antagonists and/or alpha-blocker and/or alpha-adrenoreceptor antagonists include terazosin (1-(4-amino-6,7-dimethoxy-2-quinazolinyl)-4-[(tetrahydro-2-furanyl)carbonyl]-piperazine, Chemical Abstract Services Registry Number: 63590-64-7), phenoxybenzamine (N-(2-chloroethyl)-N-(1-methyl-2-phenoxyethyl)-benzenemethanamine, synonyms: 688A; Bensylyt; Benzylyt; Dibenzylin; Dibenzyline; Dibenzyline; Phenoxybenzamine, Chemical Abstract Services Registry Number: 59-96-1), prazosin (1-(4-amino-6,7-dimethoxy-2-quinazolinyl)-4-(2-furanylcarbonyl)-piperazine, synonyms: Furazosin; Lentopres, Chemical Abstract Services Registry Number: 19216-56-9), tamsulosin (5-[(2R)-2-[[2-(2-ethoxyphenoxy)ethyl]amino]propyl]-2-methoxy-benzenesulfonamide, synonyms: Amsulosin, Chemical Abstract Services Registry Number: 106133-20-4).

Examples of suitable β3-adrenoreceptor agonists include ritobegron ([4-[2-[[(1S,2R)-2-hydroxy-2-(4-hydroxyphenyl)-1-methylethyl]amino]ethyl]-2,5-dimethylphenoxy]-Acetic acid, Chemical Abstract Services Registry Number: 255734-04-4), YM-178, solabegron (3′-[[2-[[(2R)-2-(3-chlorophenyl)-2-hydroxyethyl]amino]ethyl]amino]-1,1′-Biphenyl]-3-carboxylic acid, Chemical Abstract Services Registry Number: 252920-94-8).

Examples of suitable vanilloids and/or vanilloid receptor antagonists include resiniferatoxin (Benzeneacetic acid, 4-hydroxy-3-methoxy-, [(2S,3aR,3bS,6aR,9aR,9bR, 10R, 11aR)-3a,3b,6,6a,9a,10,11,11a-octahydro-6a-hydroxy-8,10-dimethyl-11a-(1-methylethenyl)-7-oxo-2-(phenylmethyl)-7H-2,9b-epoxyazuleno[5,4-e]-1,3-benzodioxol-5-yl]methyl ester, synonyms: Daphnetoxin, Chemical Abstract Services Registry Number: 57444-62-9).

Examples of suitable botulinum toxins include botulinum toxin A (BOTOX) (synonyms: AGN 191622; Allergan; Allergan (toxin); Botox; Botox Cosmetic; Botulin neurotoxin A; Botulin toxin A; Botulinium toxin type A; Botulinum neurotoxin A; Botulinum toxin type A; CBTX-A; Dysport; Linurase; NT 201; NT 201 (toxin); Nc 224; Nc 270; Neuronox; Oculinum; Reloxin; Vistabel; Xeomin, Chemical Abstract Services Registry Number: 93384-43-1).

In a preferred embodiment, the at least one additional pharmacologically active substance is selected from the group consisting of: “smooth muscle relaxants, bladder relaxants, bladder smooth muscle relaxants, anticholinergic agents, muscarinic acetylcholine receptor antagonists, tricyclic antidepressants, Calcium antagonist, Calcium agonist, Calcium channel blockers, Calcium channel activators, Potassium antagonists, Potassium agonists, Potassium channel blockers, Potassium channel activators, alpha-adrenergic receptor antagonists, alpha-blockers, alpha-adrenoreceptor antagonists, β3-adrenoreceptor agonists, vanilloids, vanilloid receptor antagonists, botulinum toxins”.

In another preferred embodiment, the at least one additional pharmacologically active substance is selected from the group consisting of: “oxybutynin, flavoxate, propantheline, dicyclomine, tolterodine, darifenancin, solifenancin, trospium chloride, fesoterodine, imidafenacin, PSD-506, imipramine, terodiline, terazosin, phenoxybenzamine, prazosin, tamsulosin, ritobegron, YM-178, solabegron, resiniferatoxin, botulinum toxin A (BOTOX)”.

The at least one LHRH antagonist as defined herein and, where applicable, the at least one additional pharmacologically active substance as defined herein, can be administered to various mammalian species, including human, for the herewith disclosed treatments of such mammals.

For the purpose of the present invention, all mammalian species are regarded as being comprised. Preferably, such mammals are human, domestic animals, cattle, livestock, pets, cow, sheep, pig, goat, horse, pony, donkey, hinny, mule, hare, rabbit, cat, dog, guinea pig, hamster, rat, mouse. More preferably, such mammals are human, including male human and female human. Most preferably, such mammals are male human.

The treatments of the present invention are surprisingly characterized in that the people treated do not show hormone withdrawal symptoms. It was surprisingly found that the applied doses of LHRH antagonists—in the course of the favorable dosage/administration schemes—are sufficiently low to prevent chemical (hormonal) castration, in particular testosterone castration, i.e. without effecting the undesired castration side effects (hormone withdrawal symptoms), while still achieving the desired therapeutic effects, such as significant improvement of nocturia and frequency of emptying in the course of the treatment of overactive bladder and/or detrusor overactivity as well as their different subforms and/or etiologies.

What is more, combined treatment of patients suffering from overactive bladder and/or detrusor overactivity as well as benign prostatic hyperplasia (BPH) is possible with the favorable LHRH antagonist dosage/administration schemes of the pre-sent invention.

Further, with the treatments of the present invention, prevention of other side effects, such as anticholinergic side effects, such as dry mouth, urinary retention and constipation for instance, is possible.

LHRH antagonists can be prepared for use according to the present invention as illustrated in the relevant prior art. In this connection, LHRH antagonists can be pre-sent in fast-release or slow-release (depot) formulations. Slow-release (depot) formulations are preferred in order to ensure a patient-friendly treatment scheme.

Cetrorelix, for instance, can be administered in its acetate salt form, as a reconstitute of a lyophilisates (see EP 0 611572 for preparation and process). Alternatively and preferred, it can also be applied as a slightly soluble pamoate microparticle formulation (WO 95/15767), pamoate salt (WO 02/14347) or pamoate suspension (WO 2006/069641), the latter being most preferred.

Ozarelix, for instance, can be prepared and administered as disclosed in WO 00/55190 and WO 2004/030650.

With regard to the at least one additional pharmacologically active substance, all stereoisomers are contemplated, either in a mixture or in pure or substantially pure form. The at least one additional pharmacologically active substance can have asymmetric centers at any of the carbon atoms including any one of the R radicals. Consequently, the at least one additional pharmacologically active substance can exist in the form of their racemates, in the form of the pure enantiomers and/or diastereomers or in the form of mixtures of these enantiomers and/or diastereomers. The mixtures may have any desired mixing ratio of the stereoisomers. All these different stereochemical forms and mixtures are within the scope of the present invention.

Thus, for example, the at least one additional pharmacologically active substance which has one or more centers of chirality and which occurs as racemates or as diastereomer mixtures can be fractionated by methods known per se into their optical pure isomers, i.e. enantiomers or diastereomers. The separation of the at least one additional pharmacologically active substance can take place by column separation on chiral or nonchiral phases or by recrystallization from an optionally optically active solvent or with use of an optically active acid or base or by derivatization with an optically active reagent such as, for example, an optically active alcohol, and subsequent elimination of the radical.

The at least one additional pharmacologically active substance may be present in the form of their double bond isomers as “pure” E or Z isomers, or in the form of mixtures of these double bond isomers.

Where possible, the at least one additional pharmacologically active substance may be in the form of the tautomers.

It is likewise possible for the at least one additional pharmacologically active substance to be in the form of any desired prodrugs such as, for example, esters, carbonates, carbamates, ureas, amides or phosphates, in which cases the actually biologically active form is released only through metabolism. Any compound that can be converted in vivo to provide the bioactive agent (i.e. compounds of the invention) is a prodrug within the scope and spirit of the invention.

Various forms of prodrugs are well known in the art and are described for instance in:
(i) Wermuth C G et al., Chapter 31: 671-696, The Practice of Medicinal Chemistry, Academic Press 1996;
(ii) Bundgaard H, Design of Prodrugs, Elsevier 1985; and
(iii) Bundgaard H, Chapter 5: 131-191, A Textbook of Drug Design and Development, Harwood Academic Publishers 1991.

Said references are incorporated herein by reference.

It is further known that chemical substances are converted in the body into metabolites which may where appropriate likewise elicit the desired biological effect—in some circumstances even in more pronounced form.

Any biologically active compound that was converted in vivo by metabolism from any of the at least one additional pharmacologically active substance is a metabolite within the scope and spirit of the invention.

The at least one additional pharmacologically active substance can be administered in a known manner. The route of administration may thereby be any route which effectively transports the active compound to the appropriate or desired site of action, for example orally or non-orally, in particular topically, transdermally, pulmonary, rectally, intravaginally, nasally or parenteral or by implantation. Oral administration is preferred.

The at least one additional pharmacologically active substances are converted into a form which can be administered and are mixed where appropriate with pharmaceutically acceptable carriers or diluents. Suitable excipients and carriers are described for example in Zanowiak P, Ullmann's Encyclopedia of Industrial Chemistry 2005, Pharmaceutical Dosage Forms, 1-33; Spiegel A J et al., Journal of Pharmaceutical Sciences 1963, 52: 917-927; Czetsch-Lindenwald H, Pharm. Ind. 1961, 2: 72-74; Fiedler H P, Lexikon der Hilfsstoffe für Pharmazie, Kosmetik and angrenzende Gebiete 2002, Editio Cantor Verlag, p65-68.

Oral administration can take place for example in solid form as tablet, capsule, gel capsule, coated tablet, granulation or powder, but also in the form of a drinkable solution. The at least one additional pharmacologically active substance can for oral administration be combined with known and ordinarily used, physiologically tolerated excipients and carriers such as, for example, gum arabic, talc, starch, sugars such as, for example, mannitol, methylcellulose, lactose, gelatin, surface-active agents, magnesium stearate, cyclodextrins, aqueous or nonaqueous carriers, diluents, dispersants, emulsifiers, lubricants, preservatives and flavorings (e.g. essential oils). The at least one additional pharmacologically active substance can also be dispersed in a microparticulate, e.g. nanoparticulate, composition.

Non-oral administration can take place for example by intravenous, subcutaneous, intramuscular injection of sterile aqueous or oily solutions, suspensions or emulsions, by means of implants or by ointments, creams or suppositories. Administration as sustained release form is also possible where appropriate. Implants may comprise inert materials, e.g. biodegradable polymers or synthetic silicones such as, for example, silicone rubber. Intravaginal administration is possible for example by means of vaginal rings. Intrauterine administration is possible for example by means of diaphragms or other suitable intrauterine devices. Transdermal administration is additionally provided, in particular by means of a formulation suitable for this purpose and/or suitable means such as, for example, patches.

The dosage may vary within a wide range depending on type and/or severity of the physiological and/or pathophysiological condition, the mode of administration, the age, gender, bodyweight and sensitivity of the subject to be treated. It is within the ability of a skilled worker to determine a “pharmacologically effective amount” of an LHRH antagonist of the invention and/or additional pharmacologically active substance. Administration can take place in a single dose or a plurality of separate dosages.

A suitable unit dose is, for example for the at least one additional pharmacologically active substance, from 0.001 mg to 100 mg of the active ingredient, i.e. at least one additional pharmacologically active substance, per kg of a patient's bodyweight.

### Chemical Synthesis:

### General Methods for Synthesizing the Compounds of Formula (II)

The compounds of formula (II) can be prepared for example in the following way:

Firstly, the compounds can be synthesized by preparing the depicted central tetrahydrocarbazole structure

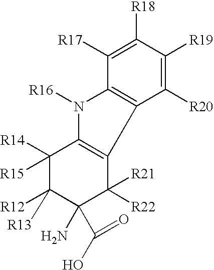

where this optionally protected tetrahydrocarbazole structure already contains the required substituents where appropriate as precursors or in protected form.

The central tetrahydrocarbazole structure is obtainable, for example, by a Fischer indole synthesis, known per se. For this purpose, a suitably substituted cyclohexanone derivative which is provided where appropriate with protective groups is condensed with the particular desired phenylhydrazine derivative which is likewise suitably substituted and, where appropriate, provided with protective groups (e.g. as described by Britten & Lockwood, J. Chem. Soc. Perkin Trans. I 1974, 1824 or Maki et al., Chem. Pharm. Bull. 1973, 21, 240). The cyclohexane structure is substituted in the 4,4′ position by the radicals —COOH and —NH₂ or where appropriate by the (protected) precursors thereof. The phenylhydrazine structure is substituted where appropriate by the radicals R17 to R20. Phenylhydrazine derivatives which are not commercially available can be prepared by processes known to the skilled worker. Positional isomers resulting where appropriate in the condensation of the cyclohexanone derivative and the phenylhydrazine derivative can be separated by chromatographic methods such as, for example, HPLC.

The derivatization of the tetrahydrocarbazole unit can in principle be achieved in various ways known to the skilled worker, and as indicated for example in WO 03/051837 or in WO 2006/005484.

The embodiments of the invention or intermediates thereof were synthesized either by conventional liquid phase synthesis in solution (see below) or else wholly or partly on a solid phase as described in WO 2006/005484.

The synthesis of relevant building blocks like tert-butyl ((S)-1-carbamoyl-2-methylbutyl)carbamate (Boc-Ile-NH₂), tert-butyl ((S)-2-methyl-1-thiocarbamoylbutyl)carbamate (Boc-Ile thioamide), (S)-2-amino-3-methylpentanamide (H-Ile thioamide), (R/S)-3-((S)-2-benzyloxycarbonylamino-3-methyl-pentanoylamino)-6,8-dichloro-2,3,4,9-tetrahydro-1H-carbazole-3-carboxylic acid (Z-(S)-11e-(R/S)-(6,8-Cl)-Thc—OH) and the synthesis of C-terminal substituted amides in solution as exemplified by the synthesis of (S)-2-{[(R/S)-3-((S)-2-benzyloxycarbonylamino-3-methyl-pentanoylamino)-6,8-dichloro-2,3,4,9-tetrahydro-1H-carbazole-3-carbonyl]amino}-3-methylpentanoic acid ((S)-Z-Ile-(R/S)-(6,8-Cl)-Thc-(S)—Ile—OH)+R1-NH—R2 has been described in WO 2006/005484.

Purification of the Crude Reaction Products (i.e. Mixtures of Diastereoisomers) by Semipreparative HPLC

Analytical and semipreparative HPLC systems from Shimadzu; column 250-50, LiChrospher® 100, RP18 (12 μm) from Merck; flow rate 60 ml/min.

Eluents:
A=970 ml of water+30 ml of ACN+1 ml of TFA
B=300 ml of water+700 ml of ACN+1 ml of TFA UV detector 220 nm.

All products were isolated by gradient elution.

The crude products are dissolved in eluent B (DMF added for products of low solubility) and purified in portions on the column (e.g. dissolve 500 mg of crude product in 15 ml of B and separate in one portion). The separation conditions in this case depend on the peptide sequence and nature and amount of the impurities and are established experimentally beforehand on the analytical column.

A typical gradient is: 60% B-100% B in 30 minutes.

If the crude products are mixtures of diastereomers, they are separated by this method.

The isolated fractions are checked by analytical HPLC. ACN and TFA are removed in a rotary evaporator, and the remaining aqueous concentrate is lyophilized.

The compounds of the present invention were prepared as indicated below The analytical characterization of the compounds of the invention took place by ¹H-NMR spectroscopy and/or mass spectrometry.

The chemicals and solvents employed were obtained commercially from usual suppliers (Acros, Avocado, Aldrich, Bachem, Fluka, Lancaster, Maybridge, Merck, Sigma, TCI etc.) or synthesized by processes known to the skilled worker.

For the exemplary embodiments indicated below, chiral building blocks were usually employed in enantiopure form. In the case of the tetrahydrocarbazole precursor, the racemic building block was employed. Final products were purified by semipreparative HPLC and characterized in the form of the pure diastereomers.

### LIST OF ABBREVIATIONS USED

e.g. for example
DBU 1,8-diazabicyclo[5.4.0]undec-7-ene
HOBt 1-hydroxybenzotriazole
Fmoc 9-fluoroenylmethoxycarbonyl
Boc tert-butyloxycarbonyl
Z benzyloxycarbonyl
Z-Cl benzyloxycarbonyl chloride
Boc₂O di-tert-butyl dicarbonate
Bzl benzyl
AA amino acid
EDT 1,2-ethanedithiol
DEAD diethyl azodicarboxylate
DIC N,N′-diisopropylcarbodiimide
DCC N,N′-dicyclohexylcarbodiimide
HATU N,N, N′,N′-tetramethyl—O-(7-azabenzotriazol-1-yl)uronium hexafluorophosphate
HOAt 1-hydroxy-7-azabenzotriazole
PyBop (benzotriazol-1-yloxy)tripyrrolidinophosphonium hexafluorophosphate
OSu N-hydroxysuccinimidyl
DIPEA diisopropyl-ethylamine
DMAP N,N′-dimethylaminopyridine
DBU 1,8-diazabicyclo[5.4.0]undec-7-ene
NMM N-methylmorpholine
TFA trifluoroacetic acid
DCM dichloromethane
DMF N,N′-dimethylformamide
DMA N,N′-dimethylacetamide
ACN acetonitrile
THF tetrahydrofuran
Me methyl
MeOH methanol
Lawesson's reagent 2,4-bis(4-methoxyphenyl)-1,3-dithia-2,4-diphosphetane 2,4-disulfide
Thc 3-amino-2,3,4,9-tetrahydro-1H-carbazole-3-carboxylic acid
Ala alanine(yl)
Val valine(yl)
Ile isoleucine(yl)
Leu leucine(yl)
Gln glutamine(yl)
Asn asparagine(yl)
Tyr tyrosine(yl)
hTyr homo-tyrosine(yl)
Arg arginine(yl)
Lys lysine(yl)
RT room temperature
m.p. melting point
ml milliliter
min minute
h hour
ELISA enzyme linked immunosorbent assay
HEPES N-(2-hydroxyethyl)piperazine-N′-2-ethanesulfonic acid
DMEM Dulbecco's modified Eagles medium
RIA radio immuno assay
LHRH luteinizing hormone releasing hormone
LH luteinizing hormone
NK1 neurokinin 1
NK2 neurokinin 2
PG protecting group

The compounds of the invention were named using the AutoNom 2000 software (ISIS™/Draw 2.5; MDL).

The contents of all cited references are hereby incorporated by reference in their entirety. The invention is explained in more detail by means of the following examples without, however, being restricted thereto.

### EXAMPLES

### Example 1

Study subjects were treated with Cetrorelix pamoate with the following doses according to the following administration schemes:
60 mg Cetrorelix pamaote administered at week 0 and 30 mg Cetrorelix pamaote administered at week 2 (treatment period) followed by up to 5 months of no treatment (treatment-free period)
60 mg Cetrorelix pamaote administered at week 0 and 60 mg Cetrorelix pamaote administered at week 2 (treatment period) followed by up to 5 months of no treatment (treatment-free period)

The results for the placebo-controlled treatments with 60+30 mg Cetrorelix pamoate and 60+60 mg Cetrorelix pamoate are illustrated below.

Table 1 shows the results for nocturia, i.e. how often the study subjects got up during the night in order to urinate. The results shown are percentage variations from baseline at the beginning of the study (week 0).

TABLE 1
Cetrorelix pamoate - Nocturia (percentage variation from baseline)

| Group | Placebo | 60 + 30 | 60 + 60 |
|---|---|---|---|
| Week 0 | 0 | 0 | 0 |
| Week 4 | −17 | −14 | −17 |
| Week 8 | −15 | −26 | −26 |
| Week 12 | −23 | −27 | −30 |
| Week 16 | −23 | −27 | −25 |
| Week 20 | −24 | −33 | −27 |
| Week 24 | −23 | −26 | −29 |

The results indicate that there is a placebo effect. However, treatments with Cetrorelix pamoate according to above doses and administration schemes demonstrate the effectiveness of the treatments of the invention. An up to 11% further decrease in nocturia frequency for the treatments of the invention could be observed compared to placebo.

Table 2 shows the results for frequency of emptying, i.e. how often the study subjects had to urinate a second time within a 2 hour test period. The results shown are percentage variations from baseline at the beginning of the study (week 0).

TABLE 2
Cetrorelix pamoate - Frequency of emptying
(percentage variation from baseline)

| Group | Placebo | 60 + 30 | 60 + 60 |
|---|---|---|---|
| Week 0 | 0 | 0 | 0 |
| Week 4 | −10 | −9 | −16 |
| Week 8 | −8 | −19 | −25 |
| Week 12 | −13 | −20 | −24 |
| Week 16 | −13 | −26 | −29 |
| Week 20 | −6 | −27 | −25 |
| Week 24 | −5 | −31 | −34 |

Again, the results indicate that there is a placebo effect. However, treatments with Cetrorelix pamoate according to above doses and administration schemes demonstrate the effectiveness of the treatments of the invention. An up to 29% further decrease in frequency for the treatments of the invention could be observed compared to placebo.

Table 3 shows the results for the IPSS irritative subscore, i.e. the sum of the effects of nocturia, frequency of emptying and urgency to urinate. The results shown are percentage variations from baseline at the beginning of the study (week 0).

TABLE 3
Cetrorelix pamoate - IPSS irritative subscore (nocturia,
frequency of emptying, urgency to urinate)

| Group | Placebo | 60 + 30 | 60 + 60 |
|---|---|---|---|
| Week 0 | 0 | 0 | 0 |
| Week 4 | −11.8 | −14.8 | −16.7 |
| Week 8 | −15.5 | −33.3 | −25 |
| Week 12 | −16.7 | −29.3 | −25 |
| Week 16 | −14.3 | −33.3 | −25 |
| Week 20 | −14.3 | −33.3 | −27.6 |
| Week 24 | −10 | −38 | −40 |

Again, the results indicate that there is a placebo effect. However, treatments with Cetrorelix pamoate according to above doses and administration schemes demonstrate the effectiveness of the treatments of the invention. An up to 30% further decrease in IPSS irritative subscore for the treatments of the invention could be observed compared to placebo.

Table 4 shows the corresponding median testosterone blood levels [ng/mL] for the treatments with Cetrorelix pamoate according to above doses and administration schemes as well as for the placebo treatment.

TABLE 4
Cetrorelix pamoate - Testosterone [ng/mL] (median)

| Group | Placebo | 60 + 30 | 60 + 60 |
|---|---|---|---|
| Week 0 | 4.23 | 3.96 | 3.71 |
| Week 4 | 4.01 | 2.59 | 2.19 |
| Week 8 | 4.30 | 4.01 | 3.70 |
| Week 12 | 4.00 | 4.10 | 3.73 |
| Week 16 | 4.09 | 3.96 | n.d. |
| Week 20 | 3.70 | 4.12 | n.d. |
| Week 24 | 3.67 | 4.30 | n.d. |

The results clearly demonstrate that chemical (hormonal) castration could be successfully prevented.

### Example 2

Study subjects were treated with Cetrorelix acetate with the following doses according to the following administration schemes:
5 mg Cetrorelix acetate administered at week 0, 1, 2 and 3 (total monthly dose of 20 mg) (treatment period) followed by up to 4 months of no treatment (treatment-free period)
10 mg Cetrorelix acetate administered at week 0 and 10 mg Cetrorelix acetate administered at week 2 (treatment period) followed by up to 4 months of no treatment (treatment-free period)
10 mg Cetrorelix acetate administered at week 0, 1, 2 and 3 (total monthly dose of 40 mg) (treatment period) followed by up to 4 months of no treatment (treatment-free period)

The results for the placebo-controlled treatments with 5+5+5+5 mg Cetrorelix acetate, 10+10 mg Cetrorelix acetate and 10+10+10+10 mg Cetrorelix acetate are illustrated below.

Table 5 shows the results for nocturia, i.e. how often the study subjects got up during the night in order to urinate. The results shown are mean values, how often the study subjects had to get up.

TABLE 5
Cetrorelix acetate - Nocturia (n-times, mean)

| Group | Placebo | 4 × 5 | 2 × 10 | 4 × 10 |
|---|---|---|---|---|
| Week 0 | 2.71 | 2.57 | 2.69 | 2.63 |
| Week 4 | 2.57 | 2.09 | 2.00 | 1.79 |
| Week 8 | 2.49 | 1.94 | 2.06 | 1.78 |
| Week 12 | 2.43 | 1.85 | 1.97 | 1.75 |
| Week 16 | 2.46 | 1.79 | 1.97 | 1.87 |
| Week 20 | 2.65 | 1.85 | 2.16 | 2.16 |

The results indicate that there is a slight placebo effect. However, treatments with Cetrorelix acetate according to above doses and administration schemes demonstrate the effectiveness of the treatments of the invention. An up to 0.71 decrease in nocturia frequency for the treatments of the invention could be observed compared to placebo.

Table 6 shows the results for frequency of emptying, i.e. how often the study subjects had to urinate a second time within a 2 hour test period. The results shown are mean values, how often the study subjects had to urinate a second time.

TABLE 6
Cetrorelix acetate - Frequency of emptying (n-times, mean)

| Group | Placebo | 4 × 5 | 2 × 10 | 4 × 10 |
|---|---|---|---|---|
| Week 0 | 2.40 | 2.49 | 2.29 | 2.86 |
| Week 4 | 2.49 | 2.23 | 1.86 | 1.62 |
| Week 8 | 2.60 | 2.00 | 1.58 | 1.66 |
| Week 12 | 2.71 | 1.73 | 1.55 | 1.81 |
| Week 16 | 2.80 | 1.52 | 1.45 | 1.65 |
| Week 20 | 2.71 | 1.79 | 1.66 | 1.78 |

The results for the treatments with Cetrorelix acetate according to above doses and administration schemes demonstrate the effectiveness of the treatments of the invention. An up to 1.35 decrease in frequency of emptying for the treatments of the invention could be observed compared to placebo.

Table 7 shows the results for the IPSS irritative subscore, i.e. the sum of the effects of nocturia, frequency of emptying and urgency to urinate. The results shown are percentage variations from baseline at the beginning of the study (week 0).

TABLE 7
Cetrorelix acetate - IPSS irritative subscore (nocturia,
frequency of emptying, urgency to urinate)

| Group | Placebo | 4 × 5 | 2 × 10 | 4 × 10 |
|---|---|---|---|---|
| Week 0 | 0 | 0 | 0 | 0 |
| Week 4 | −12.5 | −16.7 | −25 | −29.3 |
| Week 8 | −14.3 | −25 | −33.3 | −31 |
| Week 12 | 0 | −28.6 | −33.3 | −25 |
| Week 16 | 0 | −30.8 | −42.9 | −28.6 |
| Week 20 | 0 | −29.7 | −29.2 | −25 |

The results indicate that there is a slight placebo effect. However, treatments with Cetrorelix acetate according to above doses and administration schemes demonstrate the effectiveness of the treatments of the invention. An up to 43% further decrease in IPSS irritative subscore for the treatments of the invention could be observed compared to placebo.

Table 8 shows the corresponding median testosterone blood levels [ng/mL] for the treatments with Cetrorelix acetate according to above doses and administration schemes as well as for the placebo treatment.

TABLE 8
Cetrorelix acetate - Testosterone [ng/mL] (median)

| Group | Placebo | 4 × 5 | 2 × 10 | 4 × 10 |
|---|---|---|---|---|
| Week 0 | 3.93 | 3.42 | 3.27 | 3.41 |
| Week 4 | 3.18 | 2.95 | 4.41 | 2.17 |
| Week 8 | 3.39 | 3.77 | 3.73 | 3.82 |
| Week 12 | 3.06 | 4.11 | 4.45 | 4.32 |
| Week 16 | 3.77 | 3.91 | 4.16 | 4.32 |
| Week 20 | 4.02 | 3.49 | 4.12 | 4.06 |

The results clearly demonstrate that chemical (hormonal) castration could be successfully prevented.

### Example 3

Study subjects were treated with Ozarelix with the following doses according to the following administration schemes:
15 mg Ozarelix administered at week 0 and 15 mg Ozarelix administered at week 2 (treatment period) followed by up to 4 months of no treatment (treatment-free period)

The results for the placebo-controlled treatments with 15+15 mg Ozarelix are illustrated below.

Table 9 shows the results for nocturia, i.e. how often the study subjects got up during the night in order to urinate. The results shown are mean values, how often the study subjects had to get up.

TABLE 9
Ozarelix - Nocturia (n-times, mean)

| Group | Placebo | 15 + 15 |
|---|---|---|
| Week 0 | 2.92 | 2.73 |
| Week 4 | 2.33 | 1.83 |
| Week 8 | 2.04 | 1.43 |
| Week 12 | 2.17 | 1.54 |
| Week 16 | 2.17 | 1.54 |
| Week 20 | 2.26 | 1.50 |
| Week 24 | 2.23 | 1.54 |

The results indicate that there is a placebo effect. However, treatments with Ozarelix according to above doses and administration schemes demonstrate the effectiveness of the treatments of the invention. An up to 0.76 decrease in nocturia frequency for the treatments of the invention could be observed compared to placebo.

Table 10 shows the results for frequency of emptying, i.e. how often the study subjects had to urinate a second time within a 2 hour test period. The results shown are mean values, how often the study subjects had to urinate a second time.

TABLE 10
Ozarelix - Frequency of emptying (n-times, mean)

| Group | Placebo | 15 + 15 |
|---|---|---|
| Week 0 | 2.75 | 290 |
| Week 4 | 2.46 | 2.48 |
| Week 8 | 2.00 | 2.11 |
| Week 12 | 2.09 | 1.82 |
| Week 16 | 2.04 | 1.75 |
| Week 20 | 2.00 | 1.71 |
| Week 24 | 2.05 | 1.75 |

Again, the results indicate that there is a placebo effect. However, the treatments with Ozarelix according to above doses and administration schemes demonstrate the effectiveness of the treatments of the invention. An up to 0.30 decrease in frequency of emptying for the treatments of the invention could be observed compared to placebo.

Table 11 shows the results for urgency to urinate, i.e. how often the study subjects had difficulties to delay the urination. The results shown are mean values, how often the study subjects had difficulties.

TABLE 11
Ozarelix - Urgency to urinate (n-times, mean)

| Group | Placebo | 15 + 15 |
|---|---|---|
| Week 0 | 2.92 | 2.80 |
| Week 4 | 2.04 | 2.17 |
| Week 8 | 2.13 | 1.89 |
| Week 12 | 2.22 | 1.75 |
| Week 16 | 2.13 | 1.39 |
| Week 20 | 2.26 | 1.71 |
| Week 24 | 2.09 | 1.89 |

Again, the results indicate that there is a placebo effect. However, the treatments with Ozarelix according to above doses and administration schemes demonstrate the effectiveness of the treatments of the invention. An up to 0.74 decrease in urgency to urinate for the treatments of the invention could be observed compared to placebo.

Table 12 shows the corresponding median testosterone blood levels [ng/mL] for the treatments with Ozarelix according to above doses and administration schemes as well as for the placebo treatment.

TABLE 12
Ozarelix - Testosterone [ng/mL] (median)

| Group | Placebo | 15 + 15 |
|---|---|---|
| Week 0 | 3.95 | 3.57 |
| Week 4 | 4.16 | 2.97 |
| Week 8 | 3.99 | 4.19 |
| Week 12 | 3.98 | 4.11 |
| Week 16 | 4.00 | 3.82 |
| Week 20 | 3.50 | 3.92 |
| Week 24 | 4.22 | 3.86 |

The results clearly demonstrate that chemical (hormonal) castration could be successfully prevented.

### Example 4

Study subjects are treated with compound (76) with the following doses according to the following administration schemes:
in a 3 month trial, 75 mg or 150 mg compound (76) administered perorally daily as a single dose on day 2-7 of the menstrual cycle
in a 3 months trial, 75 mg, 100 mg or 150 mg compound (76) administered perorally daily as a single dose on day 2-7 of the menstrual cycle followed by a 3 months treatment-free period
in a 6 months trial, 75 mg or 150 mg compound (76) administered perorally daily as a single dose
50 mg or 150 mg per compound (76) administered daily as a single dose over 42 days (6 weeks)

The treatments with compound (76) according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

### Example 5

Study subjects are treated with compound (68) with the following doses according to the following administration schemes:
in a 3 month trial, 75 mg or 150 mg compound (68) administered perorally daily as a single dose on day 2-7 of the menstrual cycle
in a 3 months trial, 75 mg, 100 mg or 150 mg compound (68) administered perorally daily as a single dose on day 2-7 of the menstrual cycle followed by a 3 months treatment-free period
in a 6 months trial, 75 mg or 150 mg compound (68) administered perorally daily as a single dose
50 mg or 150 mg per compound (68) administered daily as a single dose over 42 days (6 weeks)

The treatments with compound (68) according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

### Example 6

Study subjects are treated with compound 36 with the following doses according to the following administration schemes:
in a 3 month trial, 75 mg or 150 mg compound 36 administered perorally daily as a single dose on day 2-7 of the menstrual cycle
in a 3 months trial, 75 mg, 100 mg or 150 mg compound 36 administered perorally daily as a single dose on day 2-7 of the menstrual cycle followed by a 3 months treatment-free period
in a 6 months trial, 75 mg or 150 mg compound 36 administered perorally daily as a single dose
50 mg or 150 mg per compound 36 administered daily as a single dose over 42 days (6 weeks)

The treatments with compound 36 according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

### Example 7

Study subjects are treated with compound 37 with the following doses according to the following administration schemes:
in a 3 month trial, 75 mg or 150 mg compound 37 administered perorally daily as a single dose on day 2-7 of the menstrual cycle
in a 3 months trial, 75 mg, 100 mg or 150 mg compound 37 administered perorally daily as a single dose on day 2-7 of the menstrual cycle followed by a 3 months treatment-free period
in a 6 months trial, 75 mg or 150 mg compound 37 administered perorally daily as a single dose
50 mg or 150 mg per compound 37 administered daily as a single dose over 42 days (6 weeks)

The treatments with compound 37 according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

### Example 8

Study subjects are treated with compound 52 with the following doses according to the following administration schemes:
in a 3 month trial, 75 mg or 150 mg compound 52 administered perorally daily as a single dose on day 2-7 of the menstrual cycle
in a 3 months trial, 75 mg, 100 mg or 150 mg compound 52 administered perorally daily as a single dose on day 2-7 of the menstrual cycle followed by a 3 months treatment-free period
in a 6 months trial, 75 mg or 150 mg compound 52 administered perorally daily as a single dose
50 mg or 150 mg per compound 52 administered daily as a single dose over 42 days (6 weeks)

The treatments with compound 52 according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

### Example 9

Study subjects are treated with compound 144 with the following doses according to the following administration schemes:
in a 3 month trial, 75 mg or 150 mg compound 144 administered perorally daily as a single dose on day 2-7 of the menstrual cycle
in a 3 months trial, 75 mg, 100 mg or 150 mg compound 144 administered perorally daily as a single dose on day 2-7 of the menstrual cycle followed by a 3 months treatment-free period
in a 6 months trial, 75 mg or 150 mg compound 144 administered perorally daily as a single dose
50 mg or 150 mg per compound 144 administered daily as a single dose over 42 days (6 weeks)

The treatments with compound 144 according to above doses and administration schemes demonstrate the effectiveness of the treatments of the invention. A decrease in in nocturia frequency, in urgency to urinate and/or frequency of emptying for the treatments of the invention can be observed compared to placebo.

The results further demonstrate that chemical (hormonal) castration can be successfully prevented.

1. A method for the treatment or prophylaxis of at least one lower urinary tract symptom in a mammal, wherein the at least one lower urinary tract symptom is selected from the group consisting of urinary incontinence, urge incontinence, overactive bladder, idiopathic overactive bladder, neurogenic overactive bladder, detrusor overactivity, idiopathic detrusor overactivity, neurogenic detrusor overactivity, benign prostatic hyperplasia (BPH), comprising administering at least one LHRH antagonist in a dose effective to treat the at least one lower urinary tract symptom and which does does not cause chemical castration in the mammal.

2. The method as claimed in claim 1, wherein the at least one lower urinary tract symptom is not associated with benign prostatic hyperplasia (BPH).

3. The method as claimed in claim 1, wherein the at least one lower urinary tract symptom is selected from the group consisting of overactive bladder, idiopathic overactive bladder, neurogenic overactive bladder, detrusor overactivity, idiopathic detrusor overactivity, and neurogenic detrusor overactivity.

4. The method as claimed in claim 1, comprising administering the at least one LHRH antagonist for the treatment or prophylaxis of at least one lower urinary tract symptom selected from the group consisting of overactive bladder, idiopathic overactive bladder, neurogenic overactive bladder, detrusor overactivity, idiopathic detrusor overactivity, and neurogenic detrusor overactivity simultaneously with benign prostatic hyperplasia (BPH).

5. The method as claimed in claim 1, wherein the at least one LHRH antagonist is selected from the group consisting of abarelix, antide, azaline B, A-75998, cetrorelix, degarelix, detirelix, ozarelix (D-63153), ganirelix, Nal-Glu-Antagonist, ramorelix, RS-68439, and teverelix.

6. The method as claimed in claim 1, wherein the at least one LHRH antagonist is cetrorelix or ozarelix (D-63153).

7. The method as claimed in claim 1, wherein the LHRH antagonist is a tetrahydrocarbazole of the formula (I) 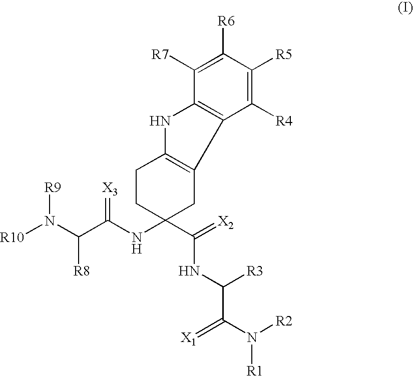
in which:
X₁ is S, O or S⁺—O⁻,
X₂ and X₃ are independently of one another O or germinally linked H₂,
R1 and R2 are independently of one another selected from the group consisting of —H, aryl, alkyl and arylalkyl radicals which are optionally substituted in the alkyl and/or aryl group by up to 3 substituents independently selected from the group consisting of —Hal, —CN and —O-alkyl,
R3 is an alkyl, arylalkyl or heteroarylalkyl radical, which are optionally substituted by up to 3 substituents independently selected from the group consisting of —Hal, —CN, —CO—O—R12, —C0-NR12R12′, —OH, —O—R13, —O—CO—R13, —O—SO₂—OR12, —O—SO₂—R12, —SO₂—OR12, —SO—R12, —O—PO(OR12)(OR12′), —O—PO(NR12R12′)₂, —O—CO—O—R13, —O—C0-NR12R12′, —O—CS—NR12R12′, —S—R12, —NR12R12′, —NH—CO—R13, —NH—SO₂—R12, —NH—CO—O—R13, —NH—CO—NHR12, —NH—C(NH)—NH₂, R4, R5, R6 and R7 are selected independently of one another from the group consisting of H, —Hal, —CN, —CONH₂, —COOH, —CF₃, —O-alkyl, —OCF₃, —NO₂, and alkyl, arylalkyl and heteroarylalkyl radicals;
R9 is a hydrogen atom, an alkyl, an aryl, a heteroaryl, an arylalkyl or a heteroarylalkyl radical;
R10 is a hydrogen atom, or the radical —R11, —CO—R11, —CO-OR11, —CO—NHR11, —C(NH)—NHR11, —SO₂—R11, or —SO₂—NHR11;
R11 is an alkyl, an aryl, a heteroaryl, an arylalkyl or a heteroarylalkyl radical, which are optionally substituted by one or more substituents independently selected from the group consisting of —Hal, —CN, -alkyl, —CF₃, —OCF₃, —OH, —O-alkyl, and —O—(CH₂CH₂—O)ₙ—CH₃;
R8 is —C₁-C₆-alkyl-aryl or —C₁-C₆-alkyl-heteroaryl, where the aryl or heteroaryl group is substituted by one to three, substituents independently selected from the group consisting of —O—(CH₂CH₂—O)ₙ—CH₃, —O—CO—R12, —O—CO—(CH₂CH₂—O)ₙ—CH₃, —O—SO₂—OR12, —O—SO₂—R12, —O—PO(OR12)(OR12′), —O—PO(NR12R12′)₂, —O—CO—OR13, —O—CO-NR12R12′, and —O—CS—NR12R12′, or,
where, however, at least
(iii) X₁ is S, or
(iv) R10 is not H, and R11 is an arylalkyl or heteroarylalkyl radical, which are substituted in the aryl or heteroaryl group by one or more substituents independently selected from the group consisting of Hal, —CN, -alkyl, —CF₃, —OCF₃, —OH, —O-alkyl, and —O—(CH₂CH₂—O)ₙ—CH₃,
R8 is an alkyl, arylalkyl or heteroarylalkyl radical, which are optionally substituted by up to 3 substituents independently selected from the group consisting of —Hal, —CN, —CO—O—R12, —CO-NR12R12′, —OH, —O—R13, —O—CO—R13, —O—SO₂—OR12, —O—SO₂—R12, —SO₂—OR12, —SO—R12, —O—PO(OR12)(OR12′), —O—PO(NR12R12′)₂, —O—CO—O—R13, —O—CO-NR12R12′, —O—CS—NR12R12′, —S—R12, —NR12R12′, —NH—CO—R13, —NH—SO₂—R12, —NH—CO—O—R13, —NH—CO—NHR12, —NH—C(NH)—NH₂;
R12 and R12′ are independently of one another H, or an alkyl, arylalkyl, aryl, heteroarylalkyl, or heteroaryl radical,
R13 is selected from an alkyl, arylalkyl, aryl, heteroarylalkyl, and heteroaryl radical, or is the group —(CH₂CH₂—O)ₙ—CH₃, and
n is an integer from 1 to 10, preferably 1 to 6.

8. The method as claimed in claim 7, wherein the at least one LHRH antagonist is selected from the group consisting of:
4-chlorobenzyl {(S)-1-[(R)-3-((S)-1-carbamoyl-2-methylpropylcarbamoyl)-6,8-dichloro-2,3,4,9-tetrahydro-1H-carbazol-3-ylcarbamoyl]-2-methylpropyl}carbamate (1),
4-chlorobenzyl {(S)-1-[(R)-3-((S)-1-carbamoyl-2-methylpropylcarbamoyl)-6,8-dichloro-2,3,4,9-tetrahydro-1H-carbazol-3-ylcarbamoyl]-2-methylbutyl}carbamate (2),
4-chlorobenzyl {(S)-1-[(R)-3-((S)-carbamoyl-2-methylbutylcarbamoyl)-6,8-dichloro-2,3,4,9-tetrahydro-1H-carbazol-3-ylcarbamoyl]-2-methylbutyl}carbamate (3),
(R)-6,8-dichloro-3-{(S)-2-[2-(2-fluorophenyl)acetylamino]-3-methyl-pentanoylamino}-2,3,4,9-tetrahydro-1H-carbazole-3-carboxylic acid ((S)-1-carbamoyl-2-methylbutyl)amide (4),
(R)-6,8-dichloro-3-{(S)-2-[2-(3-fluorophenyl)acetylamino]-3-methyl-pentanoylamino}-2,3,4,9-tetrahydro-1H-carbazole-3-carboxylic acid ((S)-1-carbamoyl-2-methylbutyl)amide (5),
2-chlorobenzyl {(S)-1-[(R)-3-((S)-carbamoyl-2-methylbutylcarbamoyl)-6,8-dichloro-2,3,4,9-tetrahydro-1H-carbazol-3-ylcarbamoyl]-2-methylbutyl}carbamate (6),
benzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-2-methylbutyl}carbamate (7),
benzyl 4-{(S)-3-benzyloxycarbonylamino-3-[(R)-3-((S)-1-carbamoyl-2-methylbutylcarbamoyl)-6,8-dichloro-2,3,4,9-tetrahydro-1H-carbazol-3-ylcarbamoyl]propyl}phenylcarbonate (8),
benzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-2-(4-phosphonooxyphenyl)ethyl]-carbamate (9),
benzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]carbamate (10),
benzyl [(S)-1-[(R)-3-((S)-carbamoyl-2-methylbutylcarbamoyl)-6,8-dichloro-2,3,4,9-tetrahydro-1H-carbazol-3-ylcarbamoyl]-3-(4-phosphonooxyphenyl)propyl]-carbamate (11),
benzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-phosphonooxyphenyl)propyl]carbamate (12),
(R)-6,8-dichloro-3-{(S)-2-[2-(2-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (13),
(R)-6,8-dichloro-3-[(S)-2-[2-(2-fluorophenyl)acetylamino]-4-(4-hydroxyphenyl)butyrylamino]-2,3,4,9-tetrahydro-1H-carbazole-3-carboxylic acid ((S)-2-methyl-1-thiocarbamoyl-butyl)amide (14),
mono(4-{(S)-3-[(R)-6,8-dichloro-3-((S)-2-methyl-1-thiocarbamoylbutylcarbamoyl)-2,3,4,9-tetrahydro-1H-carbazol-3-ylcarbamoyl]-3-[2-(2-fluorophenyl)acetylamino]-propyl}phenyl phosphate (15),
(R)-6,8-dichloro-3-{(S)-2-[3-(4-fluorophenyl)propionylamino]-3-methyl-pentanoylamino}-2,3,4,9-tetrahydro-1H-carbazole-3-carboxylic acid
((S)-1-carbamoyl-2-methylpropyl)amide (16), (S)-5-[(R)-3-((S)-1-carbamoyl-2-methylpropylcarbamoyl)-6,8-dichloro-2,3,4,9-tetrahydro-1H-carbazol-3-ylcarbamoyl]-5-[3-(4-fluorophenyl)propionylamino]pentylammonium trifluoroacetate (17),
(S)-6,8-dichloro-3-{(S)-2-[3-(2-hydroxyphenyl)propionylamino]-3-methyl-pentanoylamino}-2,3,4,9-tetrahydro-1H-carbazole-3-carboxylic acid ((S)-1-carbamoyl-2-methylbutyl)amide (18),
benzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-[4-(2-{2-[2-(2-methoxyethoxy)ethoxy]ethoxy}ethoxy)phenyl]ethyl}carbamate (19),
(R)-6,8-dichloro-3-((S)-2-{3-[2-(2-{2-[2-(2-methoxyethoxy)ethoxy]ethoxy}ethoxy)phenyl]propionylamino}-3-methyl-pentanoylamino)-2,3,4,9-tetrahydro-1H-carbazole-3-carboxylic acid ((S)-1-carbamoyl-2-methylbutyl)amide (20),
(R)-6,8-dichloro-3-((S)-2-{2-[2-(2-{2-[2-(2-methoxyethoxy)ethoxy]ethoxy}ethoxy)phenyl]acetylamino}-3-methyl-pentanoylamino)-2,3,4,9-tetrahydro-1H-carbazole-3-carboxylic acid ((S)-1-carbamoyl-2-methylbutyl)amide (21),
(R)-6,8-dichloro-3-[(S)-2-[3-(2-fluorophenyl)propionylamino]-4-(4-hydroxyphenyl)butyrylamino]-2,3,4,9-tetrahydro-1H-carbazole-3-carboxylic acid ((S)-1-carbamoyl-2-methylbutyl)amide (22),
(R)-6,8-dichloro-3-{(S)-2-[3-(2-fluorophenyl)propionylamino]-3-methyl-pentanoylamino}-2,3,4,9-tetrahydro-1H-carbazole-3-carboxylic acid
((S)-1-carbamoyl-2-methylbutyl)amide (23),
benzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-[4-(2-{2-[2-(2-methoxyethoxy)ethoxy]ethoxy}ethoxy)phenyl]propyl}carbamate (24),
benzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}carbamate (25),
3-methylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (26),
2,6-difluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (27),
3,5-difluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (28),
3,5-dichlorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (29),
3-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (30),
2-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (31),
3-chlorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (32),
3,5-difluorobenzyl {(S)-1-[(R)-3-((S)-1-carbamdyl-2-methylbutylcarbamoyl)-8-chloro-6-fluoro-2,3,4,9-tetrahydro-1H-carbazol-3-ylcarbamoyl]-2-methylbutyl}carbamate (33),
3-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (34),
2-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (35),
3-fluorobenzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-3-(4-hydroxyphenyl)propyl]-carbamate (37),
2-fluorobenzyl [(S)-1-[(R)-3-((S)-1-carbamoyl-2-methylbutylcarbamoyl)-6,8-dichloro-2,3,4,9-tetrahydro-1H-carbazol-3-ylcarbamoyl]-3-(4-hydroxyphenyl)propyl]-carbamate (38),
2-(2-fluorophenyl)ethyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}-carbamate (40),
2-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (41),
3-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (42),
2-fluorobenzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]carbamate (43),
3-fluorobenzyl [(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-3-(4-hydroxyphenyl)propyl]carbamate (45),
3-methoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (47),
4-fluorobenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (48),
2-methylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (49),
2,3-dimethoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (50),
2-methoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (51),
(R)-6,8-dichloro-3-{(S)-2-[2-(2-fluorophenyl)ethylamino]-3-methyl-pentanoylamino)-2,3,4,9-tetrahydro-1H-carbazole-3-carboxylic acid ((S)-1-carbamoyl-2-methylbutyl}amide (52),
2-trifluoromethylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (53),
3-trifluoromethylbenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (54),
3-trifluoromethoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (55),
2-trifluoromethoxybenzyl {(S)-1-[(R)-3-((S)-1-carbamoyl-2-methylbutylcarbamoyl)-6,8-dichloro-2,3,4,9-tetrahydro-1H-carbazol-3-ylcarbamoyl]-2-methylbutyl}carbamate (56),
4-fluorobenzyl {(S)-1-[(R)-6,8-dichloro-3-((S)-2-methyl-1-thiocarbamoylbutylcarbamoyl)-2,3,4,9-tetrahydro-1H-carbazol-3-ylcarbamoyl]-2-methylbutyl}carbamate (57),
(R)-6,8-dichloro-3-{(S)-2-[2-(4-fluorophenyl)ethylamino}-3-methylpentanoylamino}-2,3,4,9-tetrahydro-1H-carbazole-3-carboxylic acid ((S)-1-carbamoyl-2-methylbutyl)amide (58),
(R)-6,8-dichloro-3-{(S)-2-[2-(2,6-difluorophenyl)acetylamino]-3-methyl-pentanoylamino}-2,3,4,9-tetrahydro-1H-carbazole-3-carboxylic acid ((S)-1-carbamoyl-2-methylbutyl)amide (59),
4-fluorobenzyl {(S)-1-[(R)-8-chloro-6-fluoro-3-((S)-2-methyl-1-thiocarbamoylbutylcarbamoyl)-2,3,4,9-tetrahydro-1H-carbazol-3-ylcarbamoyl]-2-methylbutyl}-carbamate (60),
(R)-6,8-dichloro-3-{(S)-2-[2-(3-fluorophenyl)ethylamino]-3-methyl-pentanoylamino}-2,3,4,9-tetrahydro-1H-carbazole-3-carboxylic acid ((S)-1-carbamoyl-2-methylbutyl)amide (61),
(R)-8-chloro-3-{(S)-2-[2-(2,6-difluorophenyl)acetylamino]-3-methylpentanoylamino}-6-fluoro-2,3,4,9-tetrahydro-1H-carbazole-3-carboxylic acid ((S)-2-methyl-1-thiocarbamoylbutyl)amide (62),
(R)-8-chloro-6-fluoro-3-{(S)-2-[2-(4-fluorophenyl)ethylamino]-3-methyl-pentanoylamino}-2,3,4,9-tetrahydro-1H-carbazole-3-carboxylic acid ((S)-1-carbamoyl-2-methylbutyl)amide (63),
4-fluorobenzyl {(S)-1-[(R)-3-((S)-1-carbamoyl-2-methylbutylcarbamoyl)-8-chloro-6-fluoro-2,3,4,9-tetrahydro-1H-carbazol-3-ylcarbamoyl]-2-methylbutyl}carbamate (64),
(R)-8-chloro-6-fluoro-3-{(S)-2-[2-(4-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (65),
(R)-8-chloro-3-{(S)-2-[2-(2,4-difluorophenyl)acetylamino]-3-methyl-pentanoylamino}-6-fluoro-2,3,4,9-tetrahydro-1H-carbazole-3-carboxylic acid ((S)-2-methyl-1-thiocarbamoylbutyl)amide (66),
(R)-8-chloro-6-fluoro-3-{(S)-2-[2-(4-fluorophenyl)ethylamino]-3-methyl-pentanoylamino}-2,3,4,9-tetrahydro-1H-carbazole-3-carboxylic acid ((S)-2-methyl-1-thiocarbamoylbutyl)amide (67),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-2-methyl-1-thiocarbamoylbutyl)amide (68),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-1-carbamoyl-2-methylbutyl)amide (69),
(R)-3-{(S)-2-[2-(2-fluorophenyl)acetylamino]-3-methyl-pentanoylamino}-8-trifluoromethyl-2,3,4,9-tetrahydro-1H-carbazole-3-carboxylic acid ((S)-2-cyclopropyl-1-thiocarbamoylethyl)amide (70),
(R)-3-{(S)-2-[2-(2,6-difluorophenyl)acetylamino]-3-methyl-pentanoylamino}-8-trifluoromethyl-2,3,4,9-tetrahydro-1H-carbazole-3-carboxylic acid ((S)-2-cyclopropyl-1-thiocarbamoylethyl)amide (71),
(R)-8-chloro-6-fluoro-3-{(S)-2-[2-(2-fluorophenyl)acetylamino]-3-methyl-pentanoylamino}-2,3,4,9-tetrahydro-1H-carbazole-3-carboxylic acid ((S)-2-cyclopropyl-1-thiocarbamoylethyl)amide (72),
(R)-3-{(S)-2-[2-(2,6-difluorophenyl)acetylamino]-3-methylpentanoylamino}-8-trifluoromethyl-2,3,4,9-tetrahydro-1H-carbazole-3-carboxylic acid ((S)-2-methyl-1-thiocarbamoylbutyl)amide (73),
(R)-3-{(S)-2-[2-(2,6-difluorophenyl)acetylamino]-3-methyl-pentanoylamino}-8-trifluoromethyl-2,3,4,9-tetrahydro-1H-carbazole-3-carboxylic acid ((S)-1-carbamoyl-2-methylbutyl)amide (74),
(R)-3-{(S)-2-[2-(2-fluorophenyl)acetylamino]-3,-methylpentanoylamino}-8-trifluoromethyl-2,3,4,9-tetrahydro-1H-carbazole-3-carboxylic acid ((S)-1-carbamoyl-2-methylbutyl)amide (75),
(R)-3-{(S)-2-[2-(2-fluorophenyl)acetylamino]-3-methyl-pentanoylamino}-8-trifluoromethyl-2,3,4,9-tetrahydro-1H-carbazole-3-carboxylic acid ((S)-2-methyl-1-thiocarbamoylbutyl)amide (76),
(R)-8-chloro-3-{(S)-2-[2-(2,6-difluorophenyl)acetylamino]-3-methyl-pentanoylamino}-6-fluoro-2,3,4,9-tetrahydro-1H-carbazole-3-carboxylic acid ((S)-2-cyclopropyl-1-thiocarbamoylethyl)amide (77).

9. The method as claimed in claim 1, wherein the at least one LHRH antagonist is a tetrahydrocarbazole of the formula (II) 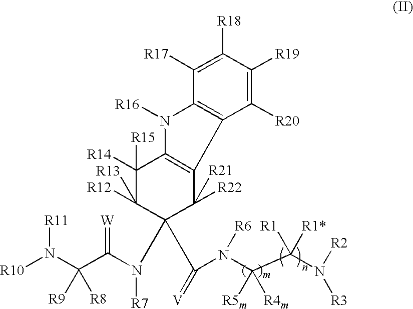
wherein:
(A) V, W are independently from each other selected from the group consisting of ═O, ═S, ═S⁺—O⁻, and germinally linked H₂;
R1, R1*—when present—together independently form ═O, ═S or ═S⁺—O⁻ or are independently both hydrogen;
R2, R3 are independently from each other selected from the group consisting of:
(i) hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1, —NX2X3, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X4, —C(O)O—X5, —C(O)NH—X6, —C(O)NX7X8, —O—X9, —O(—X10-O)ₐ—H (a=1, 2, 3, 4, 5), —O(—X11-O)_{b}—X12 (b=1, 2, 3, 4, 5), —OC(O)X13, —OC(O)—O—X14, —OC(O)NHX15, —O—C(O)NX16X17, —OP(O)(OX18)(OX19), —OSi(X20)(X21)(X22), —OS(O₂)—X23, —NHC(O)NH₂, —NHC(O)X24, —NX25C(O)X26, —NH—C(O)O—X27, —NH—C(O)NH—X28, —NH—C(O)—NX29X30, —NX31-C(O)O—X32, —NX33-C(O)—NH—X34, —NX35-C(O)NX36X37, —NHS(O₂)—X38, —NX39S(O₂)X40, —S—X41, —S(O) X42, —S(O₂)X43, —S(O₂)NH—X44, —S(O₂)NX45X46, —S(O₂)O—X47, —P(O)(OX48)(0X49), —Si(X50)(X51)(X52), —C(NH)—NH₂, —C(NX53)-NH₂, —C(NH)NHX54, —C(NH)NX55X56, —C(NX57)NHX58, —C(NX59)-—NX60X61, —NH—C(O)—NH—O—X62, —NH—C(O)NX63-O—X64, —NX65-C(O)NX66-O—X67, —N(—C(O)—NH—O—X68)₂, —N(—C(O)—NX69-O—X70)₂, —N(—C(O)NH—O—X71)(—C(O)—NX72-O—X73), —C(S)—X74, —C(S)—O—X75, —C(S)—NH—X76, —C(S)NX77X78, —C(O)NH—O—X79, —C(O)—NX80-O—X81, —C(S)NH—O—X82, —C(S)—NX83-O—X84, —C(O)—NH—NH—X85, —C(O)—NHNX₈₆X87, —C(O)—NX88-NX89X90, —C(S)—NH—NH—X91, —C(S)NHNX₉₂X93, —C(S)NX94-NX95X96, —C(O)C(O)—O—X97, —C(O)—C(O)NH₂, —C(O)—C(O)—NHX98, —C(O)—C(O)—NX99X00, —C(S)—C(O)—O—X01, —C(O—C(S)—O—X102, —C(S)—C(S)O—X103, —C(SyC(O)NH₂, —C(SyC(O)—NHX104, —C(S)C(O)—NX105X106, —C(S)C(S)—NH₂, —C(S)—C(S) NHX107, —C(S)—C(S)NX108X109, —C(O)—C(SYNH₂, —C(O)—C(S) NHX110, and —C(O)C(S)—NX111X112;
wherein X1, X2, X3, X4, X5, X6, X7, X8, X9, X10, X11, X12, X13, X14, X15, X16, X17, X18, X19, X20, X21, X22, X23, X24, X25, X26, X27, X28, X29, X30, X31, X32, X33, X34, X35, X36, X37, X38, X39, X40, X41, X42, X43, X44, X45, X46, X47, X48, X49, X50, X51, X52, X53, X54, X55, X56, X57, X58, X59, X60, X61, X62, X63, X64, X65, X66, X67, X68, X69, X70, X71, X72, X73, X74, X75, X76, X77, X78, X79, X80, X81, X82, X83, X84, X85, X86, X87, X88, X89, X90, X91, X92, X93, X94, X95, X96, X97, X98, X99, X100, X101, X102, X103, X104, X105, X106, X107, X108, X109, X110, X111, X112 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X7, X8 and/or X16, X17 and/or X29, X30 and/or X36, X37 and/or X45, X46 and/or X55, X56 and/or X60, X61 and/or X77, X78 and/or X86, X87 and/or X89, X90 and/or X92, X93 and/or X95, X96 and/or X99, X100 and/or X105, X106 and/or X108, X109 and/or X111, X112 and/or respectively together can also form heterocyclyl;
wherein optionally above substituents of substituents group (i) can in turn independently from each other be substituted with at least one substituent, identical or different, selected from the group consisting of:
(ii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX201, —NX202X203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X204, —C(O)O—X205, —C(O)NH—X206, —C(O)NX207X208, —O—X209, —O(—X210-O)C—H (c=1, 2, 3, 4, 5), —O(—X211-O)_{d}—X212 (d=1, 2, 3, 4, 5), —OC(O)—X213, —OC(O)—O—X214, —OC(O)NHX215, —O—C(O)—NX216X217, —OP(O)(OX218)(O X219), —OSi(X220)(X221)(X222), —OS(O₂)X223, —NHC(O)—NH₂, —NHC(O) X224, —NX225C(O)X226, —NH—C(O)—O—X227, —NH—C(O)—NH—X228, —NH—C(O)—NX229X230, —NX231-C(O)—O—X232, —NX233-C(O)—NH—X234, —NX235-C(O)NX236X237, —NHS(O₂)—X238, —NX239S(O₂)—X240, —S—X241, —S(O)X242, —S(O₂)X243, —S(O₂)NH—X244, —S(O₂)NX245X246, —S(O₂)O—X247, —P(O)(OX248)(OX249), Si(X₂₅₀)(X251)(X252), —C(NH)—NH₂, —C(NX253)NH₂, —C(NH)—NHX254, —C(NH)NX255X256, —C(NX257)-NHX258, —C(NX259)-—NX260X261, —NH—C(O)NH—O—X262, —NH—C(O)—NX263-O—X264, —NX265-C(O)NX266-O—X267, —N(—C(O)NH—O—X268)₂, —N(—C(O)—NX269-O—X270)₂, —N(—C(O)—NH—O—X271)(—C(O)—NX272-O—X273), —C(S)—X274, —C(S)—O—X275, —C(S)NH—X276, —C(S)NX277X278, —C(O)NH—O—X279, —C(O)—NX280-O—X281, —C(S)—NH—O—X282, —C(S)—NX283-O—X284, —C(O)—NH—NH—X285, —C(O)—NH—NX286X287, —C(O)—NX288-NX289X290, —C(S)NH—NH—X291, —C(S)—NH—NX292X293, —C(S)NX294-NX295X296, —C(O)—C(O)—O—X297, —C(O)C(O)—NH₂, —C(O)C(O)NHX298, —C(O)C(O)NX299X300, —C(S)—C(O)O—X301, —C(O)C(S)O—X302, —C(S)C(S)O—X303, —C(S)C(O)NH₂, —C(S)C(O)—NHX304, —C(S)C(O)—NX305X306, —C(S)—C(S)NH₂, —C(S)—C(S)—NHX307, —C(S)C(S)NX308X309, —C(O)—C(S)—NH₂, —C(O)C(S)NHX310, and —C(O)—C(S)—NX311X312;
wherein X201, X202, X203, X204, X205, X206, X207, X208, X209, X210, X211, X212, X213, X214, X215, X216, X217, X218, X219, X220, X221, X222, X223, X224, X225, X226, X227, X228, X229, X230, X231, X232, X233, X234, X235, X236, X237, X238, X239, X240, X241, X242, X243, X244, X245, X246, X247, X248, X249, X250, X251, X252, X253, X254, X255, X256, X257, X258, X259, X260, X261, X262, X263, X264, X265, X266, X267, X268, X269, X270, X271, X272, X273, X274, X275, X276, X277, X278, X279, X280, X281, X282, X283, X284, X285, X286, X287, X288, X289, X290, X291, X292, X293, X294, X295, X296, X297, X298, X299, X300, X301, X302, X303, X304, X305, X306, X307, X308, X309, X310, X311, X312 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl; and
wherein alternatively X207, X208 and/or X216, X217 and/or X229, X230 and/or X236, X237 and/or X245, X246 and/or X255, X256 and/or X260, X261 and/or X277, X278 and/or X286, X287 and/or X289, X290 and/or X292, X293 and/or X295, X296 and/or X299, X300 and/or X305, X306 and/or X308, X309 and/or X311, X312 and/or respectively together can also form heterocyclyl;
wherein optionally above substituents of substituents group (ii) can in turn independently from each other be substituted with at least one substituent, identical or different, selected from the group consisting of:
(iii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX401, —NX402X403, —NO₂, —OH, ═O, —OCF₃, —SH, —O—SO₃H, —OP(O)(OH)₂, —CHO, —COOH, —C(O)NH₂, SO₃H, —P(O)(OH)₂, —C(O)X404, —C(O)O—X405, —C(O)NH—X406, —C(O)NX407X408, —O—X409, —O(—X410-O)ₑ—H (e=1, 2, 3, 4, 5), —O(—X411-O)_{f}X412 (f=1, 2, 3, 4, 5), —OC(O)X413, —OC(O)O—X414, —OC(O)—NHX415, —O—C(O)—NX416X417, OP(O)(OX418)(0X419), —OSi(X420)(X421)(X422), —OS(O₂)X423, —NHC(O)—NH₂, —NHC(O)X424, —NX425C(O)—X426, —NH—C(O)—O—X427, —NH—C(O)NH—X428, —NH—C(O)NX429X430, —NX431-C(O)—O—X432, —NX433-C(O)NH—X434, —NX435-C(O) NX436X437, —NHS(O₂)X438, —NX439S(O₂)X440, —S—X441, S(O—X442, —S(O₂)X443, —S(O₂)NH—X444, —S(O₂)NX445X446,—S(O₂)O—X447, —P(O)(OX448)(0X449), —Si(X450)(X451)(X452), —C(NH)NH₂, —C(NX453)NH₂, —C(NH)NHX454, —C(NH) NX455X456, —C(NX457)NHX458, —C(NX459)NX460X461, —NH—C(O)—NH—O—X462, —NH—C(O)—NX463-O—X464, —NX465-C(O)—NX466-O—X467, —N(—C(O)—NH—O—X468)₂, —N(—C(O)—NX469-O—X470)₂, —N(—C(O)—NH—O—X471)(—C(O)NX472-O—X473), —C(S) X474, —C(S)O—X475, —C(S)—NH—X476, —C(S)NX477X478, —C(O)NH—O—X479, —C(O)NX480-O—X481, —C(S)NH—O—X482, —C(S)NX483-O—X484, —C(O)NH—NH—X485, —C(O)—NH—NX486X487, —C(O)—NX488-NX489X490, —C(S)NH—NH—X491, —C(S)NH—NX492X493, —C(S)—NX494-NX495X496, —C(O)C(O) O—X497, —C(O)—C(O)—NH₂, —C(O)C(O)NHX498, —C(O)—C(O)—NX499X500, —C(S)C(O)O—X501, —C(O)—C(S)O—X502, —C(S) C(S)—O—X503, —C(S)C(O)—NH₂, —C(S)—C(O)—NHX504, —C(S)C(O)—NX505X506, —C(S)—C(S)—NH₂, —C(S)C(S)NHX507, —C(S) C(S)—NX508X509, —C(O)C(S)NH₂, —C(O)C(S)NHX510, and —C(O)C(S)—NX511X512;
wherein X401, X402, X403, X404, X405, X406, X407, X408, X409, X410, X411, X412, X413, X414, X415, X416, X417, X418, X419, X420, X421, X422, X423, X424, X425, X426, X427, X428, X429, X430, X431, X432, X433, X434, X435, X436, X437, X438, X439, X440, X441, X442, X443, X444, X445, X446, X447, X448, X449, X450, X451, X452, X453, X454, X455, X456, X457, X458, X459, X460, X461, X462, X463, X464, X465, X466, X467, X468, X469, X470, X471, X472, X473, X474, X475, X476, X477, X478, X479, X480, X481, X482, X483, X484, X485, X486, X487, X488, X489, X490, X491, X492, X493, X494, X495, X496, X497, X498, X499, X500, X501, X502, X503, X504, X505, X506, X507, X508, X509, X510, X511, X512 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, heteroarylalkyl and
wherein alternatively X407, X408 and/or X416, X417 and/or X429, X430 and/or X436, X437 and/or X445, X446 and/or X455, X456 and/or X460, X461 and/or X477, X478 and/or X486, X487 and/or X489, X490 and/or X492, X493 and/or X495, X496 and/or X499, X500 and/or X505, X506 and/or X508, X509 and/or X511, X512 and/or respectively together can also form heterocyclyl;
n independently is 0 or 1;
with the first proviso that, if R1, R1* are not present (n is 0), R2, R3 must not both be hydrogen at the same time;
with the second proviso that, if R1, R1* are present (n is 1) and together independently form ═O, ═S or ═S⁺—O⁻ or are independently both hydrogen, R2, R3 must not both be hydrogen at the same time;
with the third proviso that, if R1, R1* are not present (n is 0), one of R2, R3 must not be hydrogen at the same time when the other one of R2, R3 is —C(═NH)—NH₂;
with the fourth proviso that, if R1, R1* are present (n is 1) and are independently both hydrogen, one of R2, R3 must not be hydrogen at the same time when the other one of R2, R3 is —C(═NH)—NH₂;
with the fifth proviso that, if R1, R1* are present (n is 1) and together independently form ═O and one of R2, R3 independently is hydrogen and the other one of R2, R3 independently is alkyl, cycloalkyl, cycloalkylalkyl, aryl, arylalkyl, and/or heteroaryl, then the other one of R2, R3 being alkyl, cycloalkyl, cycloalkylalkyl, aryl, arylalkyl, or heteroaryl must be substituted with at least one substituent selected from the group consisting of:
(iv) heterocyclyl, heterocyclylalkyl, —CF₃, —N₃, —NH₂, —NHX600, —NX601X602, —NO₂, —OH, —OCF₃, —SH, —O—SO₃H, —OP(O)(OH)₂, —CHO, —COOH, —SO₃H, —P(O)(OH)₂, —C(O)—X603, —C(O)O—X604, —C(O)NH—X605, —C(O)NX606X607, —O-aryl, —O-arylalkyl, —O-heteroaryl, —O—heteroarylalkyl, —O-heterocyclyl, —O-heterocyclylalkyl, —O(—X608-O)_{g}—H (g=1, 2, 3, 4, 5), —O(—X609-O)ₕ—X610 (h=1, 2, 3, 4, 5), —OC(O)X611, —OC(O)—O—X612, —OC(O)NHX613, —O—C(O)NX614X615, —OP(O)(OX616)(0X617), —OSi(X618)(X619)(X620), —OS(O₂)X621, —NHC(O)—X622, —NX623C(O)—X624, —NH—C(O)O—X625, —NH—C(O)—NH—X626, —NH—C(O)NX627X628, —NX629-C(O)—O—X630, —NX631-C(O)NH—X632, —NX633-C(O)NX634X635, —NHS(O₂)X636, —NX637S(O₂)X638, —S—X639, —S(O)X640, —S(O₂)X641, —S(O₂)NH—X642, —S(O₂)NX643X644, —S(O₂)O—X645, —P(O)(OX646)(OX647), —Si(X648)(X649)(X650), —C(NH)NH₂, —C(NX651)NH₂, —C(NH)NHX652, —C(NH)—NX653X654, —C(NX655)NHX656, —C(NX657)NX658X659, —NH—C(O)NH—O—X660, —NH—C(O)—NX661-O—X662, —NX663-C(O)—NX664-O—X665, —N(—C(O)NH—O—X666)₂, —N(—C(O)NX667-O—X668)₂, —N(—C(O)NH—O—X669)(—C(O)NX670-O—X671), —C(S)—X672, —C(S)—O—X673, —C(S)NH—X674, —C(S)NX675X676, —C(O)—NH—O—X677, —C(O)NX678-O—X679, —C(S)NH—O—X680, —C(S)—NX681-O—X682, —C(O)—NH—NH—X683, —C(O)NH—NX684X685, —C(O)NX686-NX687X688, —C(S)NH—NH—X689, —C(S)NH—NX690X691, —C(S)—NX692-NX693X694, —C(O)C(O)—O—X695, —C(O)C(O)—NH₂, —C(O)—C(O)NHX696, —C(O)C(O)—NX697X698, —C(S)—C(O)O—X699, —C(O)—C(S)O—X700, —C(S)—C(S)O—X701, —C(S)C(O)NH₂, —C(S)C(O)—NHX702, —C(S)C(O)NX703X704, —C(S)—C(S)NH₂, —C(S)—C(S)—NHX705, —C(S)C(S)—NX706X707, —C(O)—C(S)NH₂, —C(O)—C(S)—NHX708, and —C(O)—C(S)—NX709X710;
wherein X600, X601, X602, X603, X604, X605, X606, X607, X608, X609, X610, X611, X612, X613, X614, X615, X616, X617, X618, X619, X620, X621, X622, X623, X624, X625, X626, X627, X628, X629, X630, X631, X632, X633, X634, X635, X636, X637, X638, X639, X640, X641, X642, X643, X644, X645, X646, X647, X648, X649, X650, X651, X652, X653, X654, X655, X656, X657, X658, X659, X660, X661, X662, X663, X664, X665, X666, X667, X668, X669, X670, X671, X672, X673, X674, X675, X676, X677, X678, X679, X680, X681, X682, X683, X684, X685, X686, X687, X688, X689, X690, X691, X692, X693, X694, X695, X696, X697, X698, X699, X700, X701, X702, X703, X704, X705, X706, X707, X708, X709, X710, X711, X712 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X606, X607 and/or X614, X615 and/or X627, X628 and/or X634, X635 and/or X643, X644 and/or X653, X654 and/or X658, X659 and/or X675, X676 and/or X684, X685 and/or X687, X688 and/or X690, X691 and/or X693, X694 and/or X697, X698 and/or X703, X704 and/or X706, X707 and/or X709, X710 and/or respectively together can also form heterocyclyl;
with the further proviso that —C(O)N(alkyl)₂, —C(O)N(cycloalkyl)₂, —C(O)N(cycloalkylalkyl)₂, —C(O)N(arylalkyl)₂, —C(O)N(aryl)₂, —C(O)—N(heteroaryl)₂ are excluded from above substituents group (iv);
wherein optionally the other one of R2, R3 being alkyl, cycloalkyl, cycloalkylalkyl, aryl, arylalkyl, and/or heteroaryl can in turn independently from each other be additionally substituted with at least one substituent, identical or different, selected from above substituents group (ii);
wherein optionally the other one of R2, R3 being alkyl, cycloalkyl, cycloalkylalkyl, aryl, arylalkyl, and/or heteroaryl and being substituted with at least one substituent, identical or different, selected from above substituents group (iv) and, optionally, also (ii), can optionally be further substituted in their substituents selected from above substituents group (iv) and, optionally, also (ii), with at least one substituent, identical or different, selected from above substituents group (iii);
with the sixth proviso that, if R1, R1* are present (n is 1) and together independently form ═S or ═S⁺—O and R2, R3 are independently selected from the group consisting of hydrogen, alkyl, cycloalkyl, cycloalkylalkyl, aryl, arylalkyl, each of R2, R3 being alkyl, cycloalkyl, cycloalkylalkyl, aryl, arylalkyl must be substituted with at least one substituent selected from the group consisting of:
(v) heterocyclyl, heterocyclylalkyl, heteroaryl, heteroarylalkyl, —CF₃, —N₃, —NH₂, —NHX800, —NX801X802, —NO₂, —OH, —OCF₃, —SH, —O—SO₃H, —OP(O)(OH)₂, —CHO, —COOH, —C(O)NH₂, —SO₃H, —P(O)(OH)₂, —C(O)—X803, —C(O)O—X804, —C(O)NH—X805, —C(O)NX806X807, —O-aryl, —O-arylalkyl, —O-heteroaryl, —O-heteroarylalkyl, —O-heterocyclyl, —O-heterocyclylalkyl, —O(—X808-O)ᵢ—H (i=1, 2, 3, 4, 5), —O(—X809-O)ⱼ—X810 (j=1, 2, 3, 4, 5), —OC(O)X811, —OC(O)—O—X812, —OC(O)—NHX813, —O—C(O)—NX814X815, —OP(O)(OX816)(OX817), —OSi(X818)(X819)(X820), —OS(O₂)—X821, —NHC(O)X822, —NX823C(O)X824, —NH—C(O)O—X825, —NH—C(O)NH—X826, —NH—C(O)—NX827X828, —NX829-C(O)O—X830, —NX831-C(O)NH—X832, —NX833-C(O)—NX834X835, —NHS(O₂)X836, —NX837S(O₂)—X838, —S—X839, —S(O)X840, —S(O₂)X841, —S(O₂)NH—X842, —S(O₂)NX843X844, —S(O₂)O—X845, —P(O)(OX846)(OX847), —Si(X848)(X849)(X850), —C(NH)NH₂, —C(NX851)NH₂, —C(NH)—NHX852, —C(NH)—NX853X854, —C(NX855)NHX856, —C(NX857)-NX858X859, —NH—C(O)NH—O—X860, —NH—C(O)NX861-O—X862, —NX863-C(O)NX864-O—X865, —N(—C(O)NH—O—X866)₂, —N(—C(O)NX867-O—X868)₂, —N(—C(O)—NH—O—X869)(—C(O)—NX870-O—X871), —C(S)—X872, —C(S)—O—X873, —C(S)NH—X874, —C(S)NX875X876, —C(O)NH—O—X877, —C(O)NX878-O—X879, —C(S)—NH—O—X880, —C(S)NX881-O—X882, —C(O)—NH—NH—X883, —C(O)NH—NX884X885, —C(O)—NX886-NX887X888, —C(S)—NH—NH—X889, —C(S)NH—NX890X891, —C(S)—NX892-NX893X894, —C(O)—C(O)O—X895, —C(O)—C(O)NH₂, —C(O)C(O)—NHX896, —C(O)C(O)—NX897X898, —C(S)—C(O)—O—X899, —C(O)—C(S)—O—X900, —C(S)C(S)O—X901, —C(S)—C(O)NH₂, —C(S)—C(O)—NHX902, —C(S)C(O)NX903X904, —C(S)—C(S)—NH₂, —C(S)C(S)—NHX905, —C(S)C(S)—NX906X907, —C(O)—C(S)NH₂, —C(O)—C(S)NHX908, and —C(O)C(S)—NX909X910;
wherein X800, X801, X802, X803, X804, X805, X806, X807, X808, X809, X810, X811, X812, X813, X814, X815, X816, X817, X818, X819, X820, X821, X822, X823, X824, X825, X826, X827, X828, X829, X830, X831, X832, X833, X834, X835, X836, X837, X838, X839, X840, X841, X842, X843, X844, X845, X846, X847, X848, X849, X850, X851, X852, X853, X854, X855, X856, X857, X858, X859, X860, X861, X862, X863, X864, X865, X866, X867, X868, X869, X870, X871, X872, X873, X874, X875, X876, X877, X878, X879, X880, X881, X882, X883, X884, X885, X886, X887, X888, X889, X890, X891, X892, X893, X894, X895, X896, X897, X898, X899, X900, X901, X902, X903, X904, X905, X906, X907, X908, X909, X910, X911, X912 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X806, X807 and/or X814, X815 and/or X827, X828 and/or X834, X835 and/or X843, X844 and/or X853, X854 and/or X858, X859 and/or X875, X876 and/or X884, X885 and/or X887, X888 and/or X890, X891 and/or X893, X894 and/or X897, X898 and/or X903, X904 and/or X906, X907 and/or X909, X910 and/or respectively together can also form heterocyclyl;
wherein optionally each of R2, R3 being alkyl, cycloalkyl, cycloalkylalkyl, aryl, and/or arylalkyl can in turn independently from each other be additionally substituted with at least one substituent, identical or different, selected from above substituents group (ii);
wherein optionally each of R2, R3 being alkyl, cycloalkyl, cycloalkylalkyl, aryl, and/or arylalkyl and being substituted with at least one substituent, identical or different, selected from above substituents group (v) and, optionally, also (ii), can optionally be further substituted in their substituents selected from above substituents group (v) and, optionally, also (ii), with at least one substituent, identical or different, selected from above substituents group (iii);
m independently is 1 or 2;
R4ₘ, R5ₘ, R6, R7, R8, R9, R10, R11, R12, R13, R14, R15, R16, R17, R18, R19, R20, R21, R22 are independently from each other selected from the group consisting of:
(i) hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1001, —NX1002X1003, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)X11004, —C(O)O—X1005, —C(O)NH—X1006, —C(O)NX1007X1008, —O—X1009, —O(—X01 O—O)ₖ—H (k=1, 2, 3, 4, 5), —O(—X1011-O)I—X1012 (I=1, 2, 3, 4, 5), —OC(O)X1013, —OC(O)O—X1014, —OC(O)—NHX1015, —O—C(O)NX1016X1017, —OP(O)(OX1018)(OX1019), —OSi(X1020)(X1021)(X1022), —OS(O₂)—X1023, —NHC(O)—NH₂, —NHC(O)—X1024, —NX1025C(O)—X1026, —NH—C(O)O—X1027, —NH—C(O)—NH—X1028, —NH—C(O)NX1029X1030, —NX1031-C(O)—O—X1032, —NX1033-C(O)—NH—X1034, —NX1035-C(O)—NX1036X1037, —NHS(O₂)—X1038, —NX1039S(O₂)X1040, —S—X1041, —S(O)—X1042, —S(O₂)X1043, —S(O₂)NH—X1044, —S(O₂)NX1045X1046, —S(O₂)O—X1047, —P(O)(OX1048)(OX1049), —Si(X1050)(X1051)(X1052), —C(NH)NH₂, —C(NX1053)NH₂, —C(NH)NHX1054, —C(NH)NX1055X1056, —C(NX1057)NHX1058, —C(NX1059)NX1060X1061, —NH—C(O)NH—O—X1062, —NH—C(O)NX1063-O—X1064, —NX1065-C(O)—NX1066-O—X1067, —N(—C(O)—NH—O—X1068)₂, —N(—C(O)NX1069-O—X1070)₂, —N(—C(O)NH—O—X1071)(—C(O)NX1072-O—X1073), —C(S)—X1074, —C(S)—O—X1075, —C(S)NH—X1076, —C(S)—NX1077X1078, —C(O)—NH—O—X1079, —C(O)NX1080-O—X1081, —C(S)NH—O—X1082, —C(S)—NX1083-O—X1084, —C(O)NH—NH—X1085, —C(O)NH—NX1086X1087, —C(O)NX1088—NX1089X1090, —C(S)NH—NH—X1091, —C(S)NH—NX1092X1093, —C(S)—NX1094-NX1095X1096, —C(O)C(O)O—X1097, —C(O)C(O)NH₂, —C(O)—C(O)—NHX1098, —C(O)C(O)—NX1099X1100, —C(S)—C(O)O—X1101, —C(O)—C(S)O—X1102, —C(S)C(S)O—X1103, —C(S)—C(O)—NH₂, —C(S)—C(O)—NHX1104, —C(S)C(O)NX1105X1106, —C(S)—C(S)—NH₂, —C(S)—C(S)—NHX1107, —C(S)C(S)NX1108X1109, —C(O)—C(S)—NH₂, —C(O)—C(S)—NHX1110, and —C(O)—C(S)NX1111X112;
wherein X1001, X1002, X1003, X1004, X1005, X1006, X1007, X1008, X1009, X1010, X1011, X1012, X1013, X1014, X1015, X1016, X1017, X1018, X1019, X1020, X1021, X1022, X1023, X1024, X1025, X1026, X1027, X1028, X1029, X1030, X1031, X1032, X1033, X1034, X1035, X1036, X1037, X1038, X1039, X1040, X1041, X1042, X1043, X1044, X1045, X1046, X1047, X1048, X1049, X1050, X1051, X1052, X1053, X1054, X1055, X1056, X1057, X1058, X1059, X1060, X1061, X1062, X1063, X1064, X1065, X1066, X1067, X1068, X1069, X1070, X1071, X1072, X1073, X1074, X1075, X1076, X1077, X1078, X1079, X1080, X1081, X1082, X1083, X1084, X1085, X1086, X1087, X1088, X1089, X1090, X1091, X1092, X1093, X1094, X1095, X1096, X1097, X1098, X1099, X1100, X1101, X1102, X1103, X1104, X1105, X1106, X1107, X1108, X1109, X110, X1111, X1112 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X1007, X1008 and/or X1016, X1017 and/or X1029, X1030 and/or X1036, X1037 and/or X1045, X1046 and/or X1055, X1056 and/or X1060, X1061 and/or X1077, X1078 and/or X1086, X1087 and/or X1089, X1090 and/or X1092, X1093 and/or X1095, X1096 and/or X1099, X1100 and/or X1105, X1106 and/or X1108, X1109 and/or X1111, X1112 and/or respectively together can also form heterocyclyl;
wherein optionally above substituents of substituents group (i) can in turn independently from each other be substituted with at least one substituent, identical or different, selected from the group consisting of:
(ii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1201, —NX1202X1203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)—X1204, —C(O)O—X1205, —C(O)NH—X1206, —C(O)NX1207X1208, —O—X1209, —O(—X1210-0)ₘ—H (m=1, 2, 3, 4, 5), —O(—X1211-O)ᵣ—X1212 (n=1, 2, 3, 4, 5), —OC(O)X1213, —OC(O)—O—X1214, —OC(O)—NHX1215, —O—C(O)—NX1216X1217, —OP(O)(OX1218)(OX1219), —OSi(X1220)(X1221)(X1222), —OS(O₂)—OX1223, —NHC(O)—NH₂, —NHC(O)—X1224, —NX1225C(O)—X1226, —NH—C(O)—O—X1227, —NH—C(O)—NH—X1228, —NH—C(O)NX1229X1230, —NX1231-C(O)O—X1232, —NX1233-C(O)NH—X1234, —NX1235-C(O)—NX1236X1237, —NHS(O₂)—X1238, —NX1239S(O₂)—X1240, —S—X1241, —S(O)—X1242, —S(O₂)X1243, —S(O₂)NH—X1244, —S(O₂)NX1245X1246, —S(O₂)O—X1247, —P(O)(OX1248)(OX1249), —Si(X1250)(X1251)(X1252), —C(NH)NH₂, —C(NX1253)NH₂, —C(NH)—NHX1254, —C(NH)—NX1255X1256, —C(NX1257)NHX1258, —C(NX1259)NX1260X1261, —NH—C(O)NH—O—X1262, —NH—C(O)—NX1263-O—X1264, —NX1265-C(O)NX1266-O—X1267, —N(—C(O)NH—O—X1268)₂, —N(—C(O)—NX1269-O—X1270)₂, —N(—C(O)NH—O—X1271)(—C(O)NX1272-O—X1273), —C(S)X1274, —C(S)O—X1275, —C(S)NH—X1276, —C(S)—NX1277X1278, —C(O)NH—O—X1279, —C(O)—NX1280-O—X1281, —C(S)—NH—O—X1282, —C(S)—NX1283-O—X1284, —C(O)—NH—NH—X1285, —C(O)NH—NX1286X1287, —C(O)NX1288-NX1289X1290, —C(S)NH—NH—X1291, —C(S)—NH—NX1292X1293, —C(S)NX1294-NX1295X1296, —C(O)—C(O)O—X1297, —C(O)—C(O)—NH₂, —C(OC(O)NHX1298, —C(O)—C(O)NX1299X1300, —C(S)C(O)—O—X1301, —C(O)—C(S)—O—X1302, —C(S)—C(S)—O—X1303, —C(S)C(O)NH₂, —C(S)C(O)—NHX1304, —C(S)C(O)NX1305X1306, —C(S)C(S)—NH₂, —C(S)C(S)—NHX1307, —C(S)C(S)NX1308X1309, —C(O)—C(S)NH₂, —C(O)C(S)—NHX1310, and —C(O)C(S)NX1311X1312;
wherein X1201, X1202, X1203, X1204, X1205, X1206, X1207, X1208, X1209, X1210, X1211, X1212, X1213, X1214, X1215, X1216, X1217, X1218, X1219, X1220, X1221, X1222, X1223, X1224, X1225, X1226, X1227, X1228, X1229, X1230, X1231, X1232, X1233, X1234, X1235, X1236, X1237, X1238, X1239, X140, X1241, X1242, X1243, X1244, X1245, X1246, X1247, X1248, X1249, X1250, X1251, X1252, X1253, X1254, X1255, X1256, X1257, X1258, X1259, X1260, X1261, X1262, X1263, X1264, X1265, X1266, X1267, X1268, X1269, X1270, X1271, X1272, X1273, X1274, X1275, X1276, X1277, X1278, X1279, X1280, X1281, X1282, X1283, X1284, X1285, X1286, X1287, X1288, X1289, X1290, X1291, X1292, X1293, X1294, X1295, X1296, X1297, X1298, X1299, X1300, X1301, X1302, X1303, X1304, X1305, X1306, X1307, X1308, X1309, X1310, X1311, X1312 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X1207, X1208 and/or X1216, X1217 and/or X1229, X1230 and/or X1236, X1237 and/or X1245, X1246 and/or X1255, X1256 and/or X1260, X1261 and/or X1277, X1278 and/or X1286, X1287 and/or X1289, X1290 and/or X1292, X1293 and/or X1295, X1296 and/or X1299, X1300 and/or X1305, X1306 and/or X1308, X1309 and/or X1311, X1312 and/or respectively together can also form heterocyclyl;
wherein optionally above substituents of substituents group (ii) can in turn independently from each other be substituted with at least one substituent, identical or different, selected from the group consisting of:
(iii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NHX1401, —NX1402X1403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)—X1404, —C(O)O—X1405, —C(O)NH—X1406, —C(O)NX1407X1408, —O—X1409, —O(—X1410-O)O—H (o=1, 2, 3, 4, 5), —O(—X1411-O)ₚ—X1412 (p=1, 2, 3, 4, 5), —OC(O)—X1413, —OC(O)—O—X1414, —OC(O)—NHX1415, —O—C(O)—NX1416X1417, —OP(O)(OX1418)(0X1419), —OSi(X1420)(X1421)(X1422), —OS(O₂)—X1423, —NHC(O)—NH₂, —NHC(O)X1424, —NX1425C(O)X1426, —NH—C(O)—O—X1427, —NH—C(O)NH—X1428, —NH—C(O)NX1429X1430, —NX1431-C(O)—O—X1432, —NX1433-C(O)—NH—X1434, —NX1435-C(O)—NX1436X1437, —NHS(O₂)X1438, —NX1439S(O₂)—X1440, —S—X1441, —S(O)X1442, —S(O₂)—X1443, —S(O₂)NH—X1444, —S(O₂)NX1445X1446, —S(O₂)O—X1447, —P(O)(OX1448)(OX1449), —Si(X4450)(X451)(X1452), —C(NH)NH₂, —C(NX1453)-NH₂, —C(NH)NHX1454, —C(NH)NX1455X1456, —C(NX1457)NHX1458, —C(NX1459)NX1460X1461, —NH—C(O)NH—O—X1462, —NH—C(O)NX1463-O—X1464, —NX1465-C(O)NX1466-O—X1467, —N(—C(O)—NH—O—X1468)₂, —N(—C(O)—NX1469-O—X1470)₂, —N(—C(O)—NH—O—X1471)(—C(O)—NX1472-O—X1473), —C(S)—X1474, —C(S)—O—X1475, —C(S)NH—X1476, —C(S)NX1477X1478, —C(O)—NH—O—X1479, —C(O)NX1480-O—X1481, —C(S)NH—O—X1482, —C(S)—NX1483-O—X1484, —C(O)NH—NH—X1485, —C(O)—NH—NX1486X1487, —C(O)—NX1488-NX1489X1490, —C(S)NH—NH—X1491, —C(S)—NH—NX1492X1493, —C(S)NX1494NX1495X1496, —C(O)C(O)—O—X1497, —C(O)C(O)NH₂, —C(O)C(O)—NHX1498, —C(O)C(O)—NX1499X1500, —C(S)C(O)—O—X1501, —C(O)C(S)—O—X1502, —C(S)—C(S)—O—X1503, —C(S)—C(O)—NH₂, —C(S)—C(O)—NHX1504, —C(S)—C(O)NX1505X1506, —C(S)—C(S)—NH₂, —C(S)—C(S)NHX1507, —C(S)C(S)NX1508X1509, —C(O)—C(S)NH₂, —C(O)C(S)NHX1510, and —C(O)C(S)—NX1511X1512;
wherein X1401, X1402, X1403, X1404, X1405, X1406, X1407, X1408, X1409, X1410, X1411, X1412, X1413, X1414, X1415, X1416, X1417, X1418, X1419, X1420, X1421, X1422, X1423, X1424, X1425, X1426, X1427, X1428, X1429, X1430, X1431, X1432, X1433, X1434, X1435, X1436, X1437, X1438, X1439, X1440, X1441, X1442, X1443, X1444, X1445, X1446, X1447, X1448, X1449, X1450, X1451, X1452, X1453, X1454, X1455, X1456, X1457, X1458, X1459, X1460, X1461, X1462, X1463, X1464, X1465, X1466, X1467, X1468, X1469, X1470, X1471, X1472, X1473, X1474, X1475, X1476, X1477, X1478, X1479, X1480, X1481, X1482, X1483, X1484, X1485, X1486, X1487, X1488, X1489, X1490, X1491, X1492, X1493, X1494, X1495, X1496, X1497, X1498, X1499, X1500, X1501, X1502, X1503, X1504, X1505, X1506, X1507, X1508, X1509, X1510, X1511, X1512 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X1407, X1408 and/or X1416, X1417 and/or X1429, X1430 and/or X1436, X1437 and/or X1445, X1446 and/or X1455, X1456 and/or X1460, X1461 and/or X1477, X1478 and/or X1486, X1487 and/or X1489, X1490 and/or X1492, X1493 and/or X1495, X1496 and/or X1499, X1500 and/or X1505, X1506 and/or X1508, X1509 and/or X1511, X1512 and/or respectively together can also form “heterocyclyl;
or
(B) V, W are independently from each other selected from the group consisting of: ═O, ═S, ═S⁺—O⁻, and germinally linked H₂;
R1*, R2 together independently form heterocyclyl or together independently form heteroaryl; where heterocyclyl and heteroaryl can optionally be substituted with at least one substituent selected from below substituents group (i);
R1, R3 are independently from each other selected from the group consisting of:
(i) hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1, —NZ2Z3, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z4, —C(O)O-Z5, —C(O)NH-Z6, —C(O)NZ7Z8, —O-Z9, —O(-Z10-O)ₐ—H (a=1, 2, 3, 4, 5), —O(-Z11-O)_{b}-Z12 (b=1, 2, 3, 4, 5), —OC(O)Z13, —OC(O)—O-Z14, —OC(O)NH-Z15, —O—C(O)NZ16Z17, —OP(O)(OZ18)(OZ19), —OSi(Z20)(Z21)(Z22), —OS(O₂)-Z23, —NHC(O)—NH₂, —NHC(O)Z24, —NZ25C(O)-Z26, —NH—C(O)—O-Z27, —NH—C(O)NH-Z28, —NH—C(O)NZ29Z30, —NZ31-C(O)O-Z32, —NZ33-C(O)—NH-Z34, —NZ35-C(O)NZ36Z37, —NHS(O₂)Z38, —NZ39S(O₂)Z40, —S-Z41, —S(O)-Z42, —S(O₂)-Z43, —S(O₂)NH-Z44, —S(O₂)NZ45Z46, —S(O₂)O-Z47, —P(O)(OZ48)(0Z49), —Si(Z50)(Z51)(Z52), —C(NH)NH₂, —C(NZ53)NH₂, —C(NH)—NH-Z54, —C(NH)NZ55Z56, —C(NZ57)NH-Z58, —C(NZ59)-NZ60Z61, —NH—C(O)—NH—O-Z62, —NH—C(O)NZ63-O-Z64, —NZ65—C(O)—NZ66-O-Z67, —N(—C(O)NH—O-Z68)₂, —N(—C(O)NZ69-O-Z70)₂, —N(—C(O)NH—O-Z71)(—C(O)NZ72-O-Z73), —C(S)Z74, —C(SyO-Z75, —C(S)—NH-Z76, —C(S)—NZ77Z78, —C(O)NH—O-Z79, —C(O)NZ80-O-Z81, —C(S)NH—O-Z82, —C(S)NZ83-O-Z84, —C(O)NH—NH-Z85, —C(O)—NH—NZ86Z87, —C(O)NZ88-NZ89Z90, —C(S)—NH—NH-Z91, —C(S)—NH—NZ92Z93, —C(S)NZ94-NZ95Z96, —C(O)—C(O)O-Z97, —C(O)—C(O)—NH₂, —C(O)C(O)—NH-Z98, —C(O)C(O)NZ99Z100, —C(S)—C(O)O-Z101, —C(O)—C(S)—O-Z102, —C(S)—C(S)O-Z103, —C(S)C(O)NH₂, —C(S)—C(O)—NHZ104, —C(S)C(O)NZ105Z106, —C(S)—C(S)NH₂, —C(S)C(S)—NH-Z107, —C(S)—C(S)NZ108Z109, —C(O)C(S)NH₂, —C(O)C(S)—NHZ110, and —C(O)C(S)NZ111Z112;
wherein Z1, Z2, Z3, Z4, Z5, Z6, Z7, Z8, Z9, Z10, Z11, Z12, Z13, Z14, Z15, Z16, Z17, Z18, Z19, Z20, Z21, Z22, Z23, Z24, Z25, Z26, Z27, Z28, Z29, Z30, Z31, Z32, Z33, Z34, Z35, Z36, Z37, Z38, Z39, Z40, Z41, Z42, Z43, Z44, Z45, Z46, Z47, Z48, Z49, Z50, Z51, Z52, Z53, Z54, Z55, Z56, Z57, Z58, Z59, Z60, Z61, Z62, Z63, Z64, Z65, Z66, Z67, Z68, Z69, Z70, Z71, Z72, Z73, Z74, Z75, Z76, Z77, Z78, Z79, Z80, Z81, Z82, Z83, Z84, Z85, Z86, Z87, Z88, Z89, Z90, Z91, Z92, Z93, Z94, Z95, Z96, Z97, Z98, Z99, Z100, Z101, Z102, Z103, Z104, Z105, Z106, Z107, Z108, Z109, Z110, Z111, Z112 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively Z7, Z8 and/or Z16, Z17 and/or Z29, Z30 and/or Z36, Z37 and/or Z45, Z46 and/or Z55, Z56 and/or Z60, Z61 and/or Z77, Z78 and/or Z86, Z87 and/or Z89, Z90 and/or Z92, Z93 and/or Z95, Z96 and/or Z99, Z100 and/or Z105, Z106 and/or Z108, Z109 and/or Z111, Z112 and/or respectively together can also form heterocyclyl;
wherein optionally above substituents of substituents group (i) can in turn independently from each other be substituted with at least one substituent, identical or different, selected from the group consisting of:
(ii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z201, —NZ202Z203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z204, —C(O)O-Z205, —C(O)NH-Z206, —C(O)NZ207Z208, —O-Z209, —O(-Z210-O)C—H (c=1, 2, 3, 4, 5), —O(-Z211-O)_{d}-Z212 (d=1, 2, 3, 4, 5), —OC(O)-Z213, —OC(O)O-Z214, —OC(O)NH-Z215, —O—C(O)NZ216Z217, —OP(O)(OZ218)(0Z219), —OSi(Z220)(Z221)(Z222), —OS(O₂)Z223, —NHC(O)—NH₂, —NHC(O)-Z224, —NZ225C(O)Z226, —NH—C(O)O-Z227, —NH—C(O)—NH-Z228, NH—C(O)NZ229Z230, —NZ231-C(O)—O-Z232, —NZ233-C(O)—NH-Z234, —NZ235-C(O)—NZ236Z237, —NHS(O₂)-Z238, —NZ239S(O₂)-Z240, —S-Z241, —S(O)Z242, —S(O₂)-Z243, —S(O₂)NH-Z244, —S(O₂)NZ245Z246, —S(O₂)O-Z247, —P(O)(OZ248)(OZ249), —Si(Z250)(Z251)(Z252), —C(NH)NH₂, —C(NZ253)NH₂, —C(NH)—NH-Z254, —C(NH)NZ255Z256, —C(NZ257)-NHZ258, —C(NZ259)-NZ260Z261, —NH—C(O)NH—O-Z262, —NH—C(O)—NZ263-O-Z264, —NZ265-C(O)NZ266-O-Z267, —N(—C(O)—NH—O-Z268)₂, —N(—C(O)—NZ269-O-Z270)₂, —N(—C(O)—NH—O-Z271)(—C(O)NZ272-O-Z273), —C(S)— Z274, —C(S)—O-Z275, —C(S)—NH-Z276, —C(S)NZ277Z278, C(O)—NH—O-Z279, —C(O)NZ280-O-Z281, —C(S)—NH—O-Z282, —C(S)NZ283-O-Z284, —C(O)NH—NH-Z285, —C(O)NH-NZ286Z287, —C(O)NZ288-NZ289Z290, —C(S)NH—NH-Z291, —C(S)—NH-NZ292Z293, —C(S)NZ294-NZ295Z296, —C(O)C(O)O-Z297, —C(O)—C(O)NH₂, —C(O)—C(O)—NH-Z298, —C(O)C(O)NZ299Z300, —C(S)—C(O)O-Z301, —C(O)C(S)O-Z302, —C(S)C(S)—O-Z303, —C(S)—C(O)—NH₂, —C(S)C(O)NH-Z304, —C(S)C(O)NZ305Z306, —C(S)—C(S)NH₂, —C(S)—C(S)NH-Z307, —C(S)—C(S)—NZ308Z309, —C(O)—C(S)—NH₂, —C(O)—C(S)—NH-Z310, and —C(O)C(S)NZ311Z312”;
wherein Z201, Z202, Z203, Z204, Z205, Z206, Z207, Z208, Z209, Z210, Z211, Z212, Z213, Z214, Z215, Z216, Z217, Z218, Z219, Z220, Z221, Z222, Z223, Z224, Z225, Z226, Z227, Z228, Z229, Z230, Z231, Z232, Z233, Z234, Z235, Z236, Z237, Z238, Z239, Z240, Z241, Z242, Z243, Z244, Z245, Z246, Z247, Z248, Z249, Z250, Z251, Z252, Z253, Z254, Z255, Z256, Z257, Z258, Z259, Z260, Z261, Z262, Z263, Z264, Z265, Z266, Z267, Z268, Z269, Z270, Z271, Z272, Z273, Z274, Z275, Z276, Z277, Z278, Z279, Z280, Z281, Z282, Z283, Z284, Z285, Z286, Z287, Z288, Z289, Z290, Z291, Z292, Z293, Z294, Z295, Z296, Z297, Z298, Z299, Z300, Z301, Z302, Z303, Z304, Z305, Z306, Z307, Z308, Z309, Z310, Z311, Z312 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively Z207, Z208 and/or Z216, Z217 and/or Z229, Z230 and/or Z236, Z237 and/or Z245, Z246 and/or Z255, Z256 and/or Z260, Z261 and/or Z277, Z278 and/or Z286, Z287 and/or Z289, Z290 and/or Z292, Z293 and/or Z295, Z296 and/or Z299, Z300 and/or Z305, Z306 and/or Z308, Z309 and/or Z311, Z312 and/or respectively together can also form heterocyclyl;
wherein optionally above substituents of substituents group (ii) can in turn independently from each other be substituted with at least one substituent, identical or different, selected from the group consisting of:
(iii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z401, —NZ402Z403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z404, —C(O)O-Z405, —C(O)NH-Z406, —C(O)NZ407Z408, —O-Z409, —O(-Z410-O)ₑ—H (e=1, 2, 3, 4, 5), —O(-Z411-O)_{f}Z412 (f=1, 2, 3, 4, 5), —OC(O)-Z413, —OC(O)—O-Z414, —OC(O)—NH-Z415, —O—C(O)NZ416Z417, —OP(O)(OZ418)(0Z419), —OSi(Z420)(Z421)(Z422), —OS(O₂)Z423, —NHC(O)NH₂, —NHC(O)Z424, —NZ425C(O)Z426, —NH—C(O)—O-Z427, —NH—C(O)—NH-Z428, —NH—C(O)NZ429Z430, —NZ431-C(O)—O-Z432, —NZ433-C(O)NH-Z434, —NZ435-C(O)—NZ436Z437, —NHS(O₂)Z438, —NZ439S(O₂)Z440, —S-Z441, —S(O)Z442, —S(O₂)Z443, —S(O₂)NH-Z444, —S(O₂)NZ445Z446, —S(O₂)O-Z447, —P(O)(OZ448)(0Z449), —Si(Z450)(Z451)(Z452), —C(NH)NH₂, —C(NZ453)NH₂, —C(NH)NH-Z454, —C(NH)—NZ455Z456, —C(NZ457)NH-Z458, —C(NZ459)NZ460Z461, —NH—C(O)—NH—O-Z462, —NH—C(O)—NZ463-O-Z464, —NZ465-C(O)—NZ466-O-Z467, —N(—C(O)NH—O-Z468)₂, —N(—C(O)—NZ469-O-Z470)₂, —N(—C(O)—NH—O-Z471)(—C(O)—NZ472-O-Z473), —C(S)-Z474, —C(S)O-Z475, —C(S)—NH-Z476, —C(S)—NZ477Z478, —C(O)—NH—O-Z479, —C(O)NZ480-O-Z481, —C(S)—NH—O-Z482, —C(S)—NZ483-O-Z484, —C(O)—NH—NH-Z485, —C(O)—NH-NZ486Z487, —C(O)—NZ488-NZ489Z490, —C(S)NH—NH-Z491, —C(S)NH—NZ492Z493, —C(S)NZ494-NZ495Z496, —C(O)—C(O)O-Z497, —C(O)—C(O)NH₂, —C(O)C(O)—NH-Z498, —C(O)—C(O)—NZ499Z500, —C(S)—C(O)—O-Z501, —C(O)—C(S)O-Z502, —C(S)C(S)O-Z503, —C(S)C(O)—NH₂, —C(S)C(O)—NH-Z504, —C(S)C(O)—NZ505Z506, —C(S)—C(S)NH₂, —C(S)C(S)NH-Z507, —C(S)C(S)NZ508Z509, —C(O)—C(S)NH₂, —C(O)—C(S)—NH-Z510, and —C(O)C(S)—NZ511Z512;
wherein Z401, Z402, Z403, Z404, Z405, Z406, Z407, Z408, Z409, Z410, Z411, Z412, Z413, Z414, Z415, Z416, Z417, Z418, Z419, Z420, Z421, Z422, Z423, Z424, Z425, Z426, Z427, Z428, Z429, Z430, Z431, Z432, Z433, Z434, Z435, Z436, Z437, Z438, Z439, Z440, Z441, Z442, Z443, Z444, Z445, Z446, Z447, Z448, Z449, Z450, Z451, Z452, Z453, Z454, Z455, Z456, Z457, Z458, Z459, Z460, Z461, Z462, Z463, Z464, Z465, Z466, Z467, Z468, Z469, Z470, Z471, Z472, Z473, Z474, Z475, Z476, Z477, Z478, Z479, Z480, Z481, Z482, Z483, Z484, Z485, Z486, Z487, Z488, Z489, Z490, Z491, Z492, Z493, Z494, Z495, Z496, Z497, Z498, Z499, Z500, Z501, Z502, Z503, Z504, Z505, Z506, Z507, Z508, Z509, Z510, Z511, Z512 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively Z407, Z408 and/or Z416, Z417 and/or Z429, Z430 and/or Z436, Z437 and/or Z445, Z446 and/or Z455, Z456 and/or Z460, Z461 and/or Z477, Z478 and/or Z486, Z487 and/or Z489, Z490 and/or Z492, Z493 and/or Z495, Z496 and/or Z499, Z500 and/or Z505, Z506 and/or Z508, Z509 and/or Z511, Z512 and/or respectively together can also form heterocyclyl;
alternatively, R1, R3 can also independently from each other be no substituent;
n independently is 1;
m independently is 1 or 2;
R4ₘ, R5ₘ, R6, R7, R8, R9, R10, R11, R12, R13, R14, R15, R16, R17, R18, R19, R20, R21, R22 are independently from each other selected from the group consisting of:
(i) hydrogen, alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1001, —NZ1002Z1003, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)Z1004, —C(O)O-Z1005, —C(O)NH-Z1006, —C(O)NZ1007Z1008, —O-Z1009, —O(-Z1110-O)ₖ—H (k=1, 2, 3, 4, 5), —O(-Z1011—O)ᵣ-Z1012 (I=1, 2, 3, 4, 5), —OC(O)Z1013, —OC(O)—O-Z1014, —OC(O)—NH-Z1015, —O—C(O)NZ1016Z1017, —OP(O)(OZ1018)(OZ1019), —OSi(Z1020)(Z1021)(Z1022), —OS(O₂)-Z1023, —NHC(O)—NH₂, —NHC(O)-Z1024, —NZ1025C(O)Z1026, —NH—C(O)—O-Z1027, —NH—C(O)—NH-Z1028, —NH—C(O)NZ1029Z1030, —NZ1031-C(O)O-Z1032, —NZ1033-C(O)—NH-Z1034, —NZ1035-C(O)NZ1036Z1037, —NHS(O₂)Z1038, —NZ1039S(O₂)-Z1040, —S-Z1041, —S(O)Z1042, —S(O₂)-Z1043, —S(O₂)NH-Z1044, —S(O₂)NZ1045Z1046, —S(O₂)O-Z1047, —P(O)(OZ1048)(OZ1049), —Si(Z1050)(Z1051)(Z1052), —C(NH)—NH₂, —C(NZ1053)NH₂, —C(NH)NH-Z1054, —C(NH)NZ1055Z1056, —C(NZ1057)-NH-Z1058, —C(NZ1059)-NZ1060Z1061, —NH—C(O)—NH—O-Z1062, —NH—C(O)—NZ1063-O-Z1064, —NZ1065-C(O)—NZ1066-O-Z1067, —N(—C(O)—NH—O-Z1068)₂, —N(—C(O)NZ1069-O-Z1070)₂, —N(—C(O)NH—O-Z1071)(—C(O)NZ1072-O-Z1073), —C(S)Z1074, —C(S)—O-Z1075, —C(S)—NH-Z1076, —C(S)—NZ1077Z1078, —C(O)—NH—O-Z1079, —C(O)NZ1080-O-Z1081, —C(S)NH—O-Z1082, —C(S)—NZ1083-O-Z1084, —C(O)NH—NH-Z1085, —C(O)NH-NZ1086Z1087, —C(O)—NZ1088-NZ1089Z1090, —C(S)NH—NH-Z1091, —C(S)NH-NZ1092Z1093, —C(S)NZ1094-NZ1095Z1096, —C(O)C(O)—O-Z1097, —C(O)C(O)NH₂, —C(O)—C(O)—NH-Z1098, —C(O)—C(O)NZ1099Z1100, —C(S)C(O)O-Z1101, —C(O)—C(S)O-Z1102, —C(S)—C(S)—O-Z1103, —C(S)C(O)NH₂, —C(S)C(O)—NH-Z1104, —C(S)—C(O)—NZ1105Z1106, —C(S)C(S)—NH₂, —C(S)C(S)—NH-Z1107, —C(S)C(S)—NZ1108Z1109, —C(O)C(S)NH₂, —C(O)—C(S)—NH-Z1110, and —C(O)C(S)—NZ1111Z1112;
wherein X1001, X1002, X1003, X1004, X1005, X1006, X1007, X1008, X1009, X1010, X1011, X1012, X1013, X1014, X1015, X1016, X1017, X1018, X1019, X1020, X1021, X1022, X1023, X1024, X1025, X1026, X1027, X1028, X1029, X1030, X1031, X1032, X1033, X1034, X1035, X1036, X1037, X1038, X1039, X1040; X1041, X1042, X1043, X1044, X1045, X1046, X1047, X1048, X1049, X1050, X1051, X1052, X1053, X1054, X1055, X1056, X1057, X1058, X1059, X1060, X1061, X1062, X1063, X1064, X1065, X1066, X1067, X1068, X1069, X1070, X1071, X1072, X1073, X1074, X1075, X1076, X1077, X1078, X1079, X1080, X1081, X1082, X1083, X1084, X1085, X1086, X1087, X1088, X1089, X1090, X1091, X1092, X1093, X1094, X1095, X1096, X1097, X1098, X1099, X1100, X1101, X1102, X1103, X1104, X1105, X1106, X1107, X1108, X1109, X1110, X1111, X1112 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively X1007, X1008 and/or X1016, X1017 and/or X1029, X1030 and/or X1036, X1037 and/or X1045, X1046 and/or X1055, X1056 and/or X1060, X1061 and/or X1077, X1078 and/or X1086, X1087 and/or X1089, X1090 and/or X1092, X1093 and/or X1095, X1096 and/or X1099, X1100 and/or X1105, X1106 and/or X1108, X1109 and/or X1111, X1112 and/or respectively together can also form heterocyclyl;
wherein optionally above substituents of substituents group (i) can in turn independently from each other be substituted with at least one substituent, identical or different, selected from the group consisting of:
(ii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1201, —NZ1202Z1203, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)-Z1204, —C(O)O-Z1205, —C(O)NH-Z1206, —C(O)NZ1207Z1208, —O-Z1209, —O(-Z1210-O)ₘ—H (m=1, 2, 3, 4, 5), —O(-Z1211-O)ₙ-Z1212 (n=1, 2, 3, 4, 5), —OC(O)-Z1213, —OC(O)—O—Z1214, —OC(O)NH-Z1215, —O—C(O)—NZ1216Z1217, —OP(O)(OZ1218)(OZ1219), —OSi(Z1220)(Z1221)(Z1222), —OS(O₂)-Z1223, —NHC(O)NH₂, —NHC(O)-Z1224, —NZ1225C(O)Z1226, —NH—C(O)O-Z1227, —NH—C(O)NH-Z1228, —NH—C(O)—NZ1229Z1230, —NZ1231-C(O)O-Z1232, —NZ1233-C(O)NH-Z1234, —NZ1235-C(O)—NZ1236Z1237, —NHS(O₂)Z1238, —NZ1239S(O₂)Z1240, —S-Z1241, —S(O)Z1242, —S(O₂)Z1243, —S(O₂)NH-Z1244, —S(O₂)NZ1245Z1246, —S(O₂)O-Z1247, —P(O)(OZ1248)(OZ1249), —Si(Z1250)(Z1251)(Z1252), —C(NH)NH₂, —C(NZ1253)NH₂, —C(NH)NH-Z1254, —C(NH)—NZ1255Z1256, —C(NZ1257)NH-Z1258, —C(NZ1259)NZ1260Z1261, —NH—C(O)NH—O-Z1262, —NH—C(O)—NZ1263-O-Z1264, —NZ1265-C(O)—NZ1266-O-Z1267, —N(—C(O)—NH—O-Z1268)₂, —N(—C(O)—NZ1269-O-Z1270)₂, —N(—C(O)—NH—O-Z1271)(—C(O)—NZ1272-O-Z1273), —C(S)-Z1274, —C(S)—O-Z1275, —C(S)NH-Z1276, —C(S) NZ1277Z1278, —C(O)—NH—O-Z1279, —C(O)—NZ1280-O-Z1281, —C(S)NH—O-Z1282, —C(S)NZ1283-O-Z1284, —C(O)NH—NH-Z1285, —C(O)NH-NZ1286Z1287, —C(O)NZ1288-NZ1289Z1290, —C(S)NH—NH-Z1291, —C(S)NH-NZ1292Z1293, —C(S)NZ1294-NZ1295Z1296, —C(O)C(O)O-Z1297, —C(O)C(O)NH₂, —C(O)C(O)—NH-Z1298, —C(O)—C(O)—NZ1299Z1300, —C(S)C(O)O-Z1301, —C(O)C(S—O-Z1302, —C(S)C(S)O-Z1303, —C(S)C(O)NH₂, —C(S)—C(O)—NH-Z1304, —C(S)—C(O)NZ1305Z1306, —C(S)—C(S)—NH₂, —C(S)C(S)—NH-Z1307, —C(S)—C(S)—NZ1308Z1309, —C(O)—C(S)—NH₂, —C(O)C(S)—NH-Z1310, and —C(O)C(S)—NZ1311Z1312;
wherein Z1201, Z1202, Z1203, Z1204, Z1205, Z1206, Z1207, Z1208, Z1209, Z1210, Z1211, Z1212, Z1213, Z1214, Z1215, Z1216, Z1217, Z1218, Z1219, Z1220, Z1221, Z1222, Z1223, Z1224, Z1225, Z1226, Z1227, Z1228, Z1229, Z1230, Z1231, Z1232, Z1233, Z1234, Z1235, Z1236, Z1237, Z1238, Z1239, Z1240, Z1241, Z1242, Z1243, Z1244, Z1245, Z1246, Z1247, Z1248, Z1249, Z1250, Z1251, Z1252, Z1253, Z1254, Z1255, Z1256, Z1257, Z1258, Z1259, Z1260, Z1261, Z1262, Z1263, Z1264, Z1265, Z1266, Z1267, Z1268, Z1269, Z1270, Z1271, Z1272, Z1273, Z1274, Z1275, Z1276, Z1277, Z1278, Z1279, Z1280, Z1281, Z1282, Z1283, Z1284, Z1285, Z1286, Z1287, Z1288, Z1289, Z1290, Z1291, Z1292, Z1293, Z1294, Z1295, Z1296, Z1297, Z1298, Z1299, Z1300, Z1301, Z1302, Z1303, Z1304, Z1305, Z1306, Z1307, Z1308, Z1309, Z1310, Z1311, Z1312 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively Z1207, Z1208 and/or Z1216, Z1217 and/or Z1229, Z1230 and/or Z1236, Z1237 and/or Z1245, Z1246 and/or Z1255, Z1256 and/or Z1260, Z1261 and/or Z1277, Z1278 and/or Z1286, Z1287 and/or Z1289, Z1290 and/or Z1292, Z1293 and/or Z1295, Z1296 and/or Z1299, Z1300 and/or Z1305, Z1306 and/or Z1308, Z1309 and/or Z1311, Z1312 and/or respectively together can also form heterocyclyl;
wherein optionally above substituents of substituents group (ii) can in turn independently from each other be substituted with at least one substituent, identical or different, selected from the group consisting of:
(iii) alkyl, (C₉-C₃₀)alkyl, cycloalkyl, cycloalkylalkyl, heterocyclyl, heterocyclylalkyl, aryl, arylalkyl, heteroaryl, heteroarylalkyl, —F, —Cl, —Br, —I, —CN, —CF₃, —N₃, —NH₂, —NH-Z1401, —NZ1402Z1403, —NO₂, —OH, ═O, —OCF₃, —SH, —O—SO₃H, —OP(O)(OH)₂, —CHO, —COOH, —C(O)NH₂, —SO₃H, —P(O)(OH)₂, —C(O)-Z1404, —C(O)O-Z1405, —C(O)NH-Z1406, —C(O)NZ1407Z1408, —O-Z1409, —O(-Z1410-O)ₒ—H (o=1, 2, 3, 4, 5), —O(-Z1411-O)ₚ-Z1412 (p=1, 2, 3, 4, 5), —OC(O)-Z1413, —OC(O)—O-Z1414, —OC(O)NH-Z1415, —O—C(O)—NZ1416Z1417, —OP(O)(OZ1418)(0Z1419), OSi(Z1420)(Z1421)(Z1422), —PS(O₂)-Z1423, —NHC(O)—NH₂, —NHC(O)-Z1424, —NZ1425C(O)Z1426, —NH—C(O)O-Z1427, —NHC(O)NH-Z1428, —NH—C(O)NZ1429Z1430, —NZ1431-C(O)OZ1432, —NZ1433-C(O)NH-Z1434, —NZ1435-C(O)—NZ1436Z1437, —NHS(O₂)Z1438, —NZ1439S(O₂)Z1440, —S-Z1441, —S(O)Z1442, —S(O₂)Z11443, —S(O₂)NH-Z1444, —S(O₂)NZ1445Z1446, —S(O₂)O-Z1447, —P(O)(OZ1448)(OZ1449), —Si(Z1450)(Z1451)(Z1452), —C(NH)—NH₂, —C(NZ1453)-NH₂, —C(NH)NH-Z1454, —C(NH)—NZ1455Z1456, —C(NZ1457)NH-Z1458, —C(NZ1459) NZ1460Z1461, —NH—C(O)—NH—O-Z1462, —NH—C(O)NZ1463-O-Z1464, —NZ1465-C(O)NZ1466-O-Z1467, —N(—C(O)—NH—O—Z1468)₂, —N(—C(O)NZ1469-O-Z1470)₂, —N(—C(O)NH—O-Z1471)(—C(O)NZ1472-O-Z1473), —C(S)-Z1474, —C(S)O-Z1475, —C(S)NH-Z1476, —C(S)NZ1477Z1478, —C(O)NH—O-Z1479, —C(O)—NZ1480-O-Z1481, —C(S)—NH—O-Z1482, —C(S)NZ1483-O-Z1484, —C(O)NH—NH-Z1485; —C(O)NH-NZ1486Z1487, —C(O) NZ1488-NZ1489Z1490, —C(S)NH—NH-Z1491, —C(S)—NH-NZ1492Z1493, —C(S)NZ1494-NZ1495Z1496, —C(O)C(O—OZ1497, —C(O)C(O)NH₂, —C(O)C(O)—NHZ1498, —C(O)—C(O)—NZ1499Z1500, —C(S)—C(O)O-Z1501, —C(O)C(S)—O-Z1502, —C(S)C(S)O-Z1503, —C(S)—C(O)—NH₂, —C(S)—C(O)—NH-Z1504, —C(S)C(O)—NZ1505Z1506, —C(S)C(S)—NH₂, —C(S)C(S)—NH-Z1507, —C(S)—C(S)—NZ1508Z1509, —C(O)C(S)—NH₂, —C(O) C(S)NH-Z1510, and —C(O)—C(S)—NZ1511Z1512;
wherein Z1401, Z1402, Z1403, Z1404, Z1405, Z1406, Z1407, Z1408, Z1409, Z1410, Z1411, Z1412, Z1413, Z1414, Z1415, Z1416, Z1417, Z1418, Z1419, Z1420, Z1421, Z1422, Z1423, Z1424, Z1425, Z1426, Z1427, Z1428, Z1429, Z1430, Z1431, Z1432, Z1433, Z1434, Z1435, Z1436, Z1437, Z1438, Z1439, Z1440, Z1441, Z1442, Z1443, Z1444, Z1445, Z1446, Z1447, Z1448, Z1449, Z1450, Z1451, Z1452, Z1453, Z1454, Z1455, Z1456, Z1457, Z1458, Z1459, Z1460, Z1461, Z1462, Z1463, Z1464, Z1465, Z1466, Z1467, Z1468, Z1469, Z1470, Z1471, Z1472, Z1473, Z1474, Z1475, Z1476, Z1477, Z1478, Z1479, Z1480, Z1481, Z1482, Z1483, Z1484, Z1485, Z1486, Z1487, Z1488, Z1489, Z1490, Z1491, Z1492, Z1493, Z1494, Z1495, Z1496, Z1497, Z1498, Z1499, Z1500, Z1501, Z1502, Z1503, Z1504, Z1505, Z1506, Z1507, Z1508, Z1509, Z1510, Z1511, Z1512 are independently from each other selected from the group consisting of: alkyl, (C₉-C₃₀)alkyl, cycloalkyl, cycloalkylalkyl, heterocyclyl, heterocyclylalkyl, aryl, arylalkyl, heteroaryl, and heteroarylalkyl and
wherein alternatively Z1407, Z1408 and/or Z1416, Z1417 and/or Z1429, Z1430 and/or Z1436, Z1437 and/or Z1445, Z1446 and/or Z1455, Z1456 and/or Z1460, Z1461 and/or Z1477, Z1478 and/or Z1486, Z1487 and/or Z1489, Z1490 and/or Z1492, Z1493 and/or Z1495, Z1496 and/or Z1499, Z1500 and/or Z1505, Z1506 and/or Z1508, Z1509 and/or Z1511, Z1512 and/or respectively together can also form heterocyclyl.

10. The method as claimed in claim 9, wherein the at least one LHRH antagonist is selected from the group consisting of:
Compound 1 ((S)-1-{(R3-[(R)-1-(5-Amino-[1,3,4]oxadiazol-2-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester 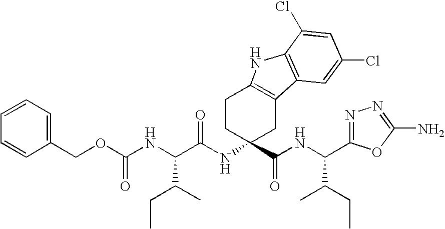
Compound 2 ((S)-1-{(S)-3-[(R)-1-(5-Amino-[1,3,4]oxadiazol-2-yl)-2-methylbutyl-carbamoyl]-6,8-dichloro-2,3,4,9-tetrahydro-1H-carbazol-3-ylcarbamoyl}-2-methyl-butyl)-carbamic acid benzyl ester 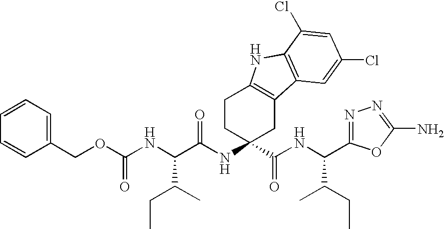
Compound 3 ((S)-1-{(R)-6,8-Dichloro-3-[(S)-2-methyl-1-(3-methyl-[1,2,4]oxadiazol-5-yl)-butylcarbamoyl]-2,3,4,9-tetrahydro-1H-carbazol-3-ylcarbamoyl}-2-methyl-butyl)-carbamic acid benzyl ester 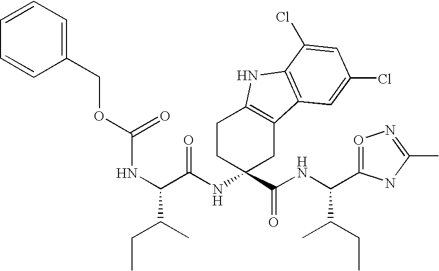
Compound 4 ((S)-1-{(S)-6,8-Dichloro-3-[(S)-2-methyl-1-(3-methyl-[1,2,4]oxadiazol-5-yl)-butylcarbamoyl]-2,3,4,9-tetrahydro-1H-carbazol-3-ylcarbamoyl}-2-methyl-butyl)-carbamic acid benzyl ester 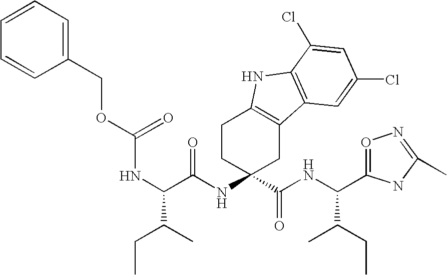
Compound 5 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid ethyl ester 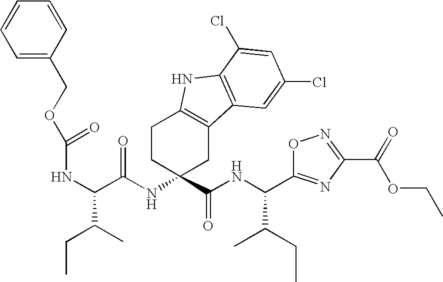
Compound 6 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid ethyl ester 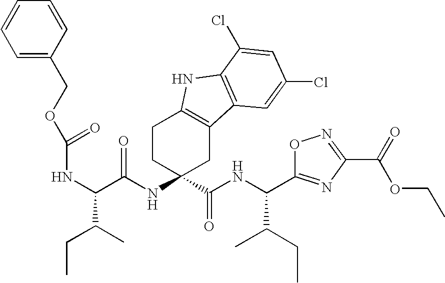
Compound 7 ((S)-1-{(S)-6,8-Dichloro-3-[(S)-2-methyl-1-(5-oxo-4,5-dihydro-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester 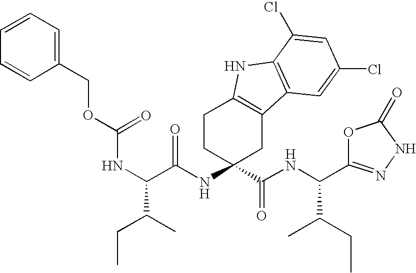
Compound 8 {(S)-1-[(R)-6,8-Dichloro-3-((S)-2-methyl-1-[1,3,4]oxadiazol-2-yl-butylcarbamoyl)-2,3,4,9-tetrahydro-1H-carbazol-3-ylcarbamoyl]-2-methyl-butyl}-carbamic acid benzyl ester 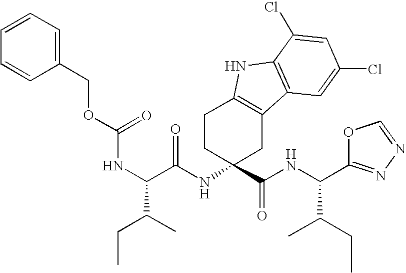
Compound 9 {(S)-1-[(S)-6,8-Dichloro-3-((S)-2-methyl-1-[1,3,4]oxadiazol-2-yl-butylcarbamoyl)-2,3,4,9-tetrahydro-1H-carbazol-3-ylcarbamoyl]-2-methyl-butyl}-carbamic acid benzyl ester 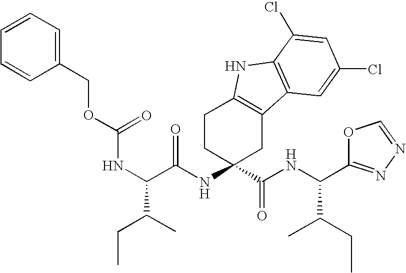
Compound 10 ((S)-1-{(R)-3-[(S)-1-(3-Carbamoyl-[1,2,4]oxadiazol-5-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester 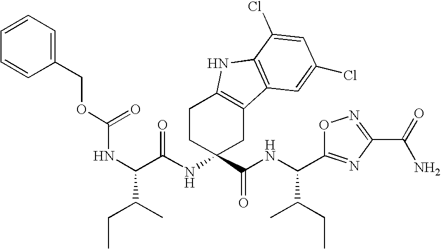
Compound 11 ((S)-1-{(S)-3-[(S)-1-[3-Carbamoyl-(1,2,4]oxadiazol-5-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester 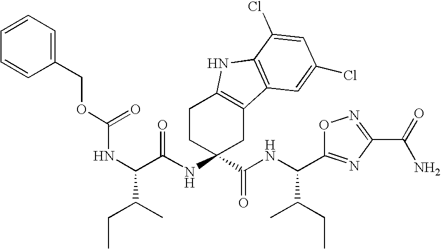
Compound 12 5-{(S)-1-[((R)-3-{(S)-2-[2-(2,6-Difluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester 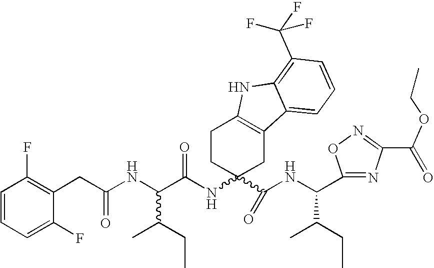
Compound 13 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,3,4]oxadiazole-2-carboxylic acid ethyl ester 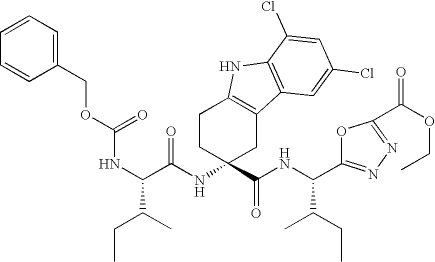
Compound 14 ((S)-1-{(R)-3-[(S)-1-(5-Acetylamino-[1,3,4]oxadiazol-2-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester 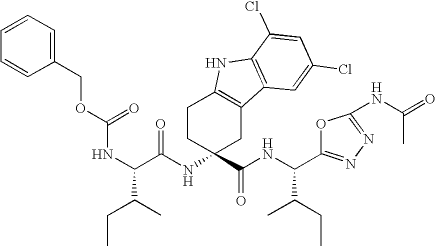
Compound 15 5-((S)-1-{([(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,3,4]oxadiazole-2-carboxylic acid ethyl ester 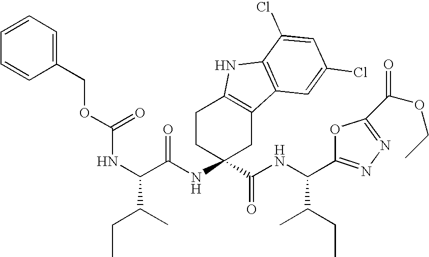
Compound 16 ((S)-1-{(S)-3-[(S)-1-(5-Acetylamino-[1,3,4]oxadiazol-2-yl)-2-methyl-butylcarbamoyl]-6,8-dichloro-2,3,4,9-tetrahydro-1H-carbazol-3-ylcarbamoyl}-2-methyl-butyl)-carbamic acid benzyl ester 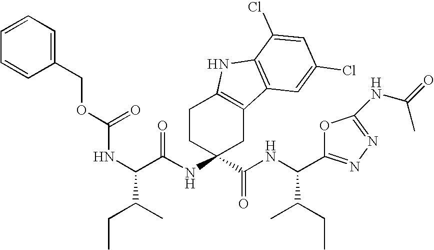
Compound 17 ((S)-1-{(R)-6,8-Dichloro-3-[(S)-2-methyl-1-(5-oxo-4,5-dihydro-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester 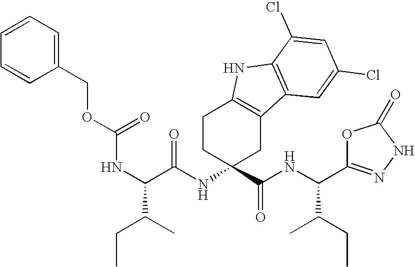
Compound 18 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid propyl ester 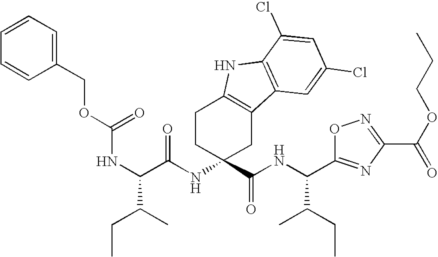
Compound 19 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid propyl ester 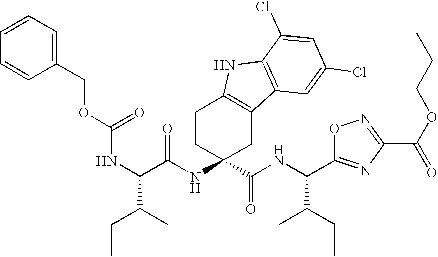
Compound 20 ((S)-1-{(R)-6,8-Dichloro-3-[(S)-1-(3-diethylcarbamoyl-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester 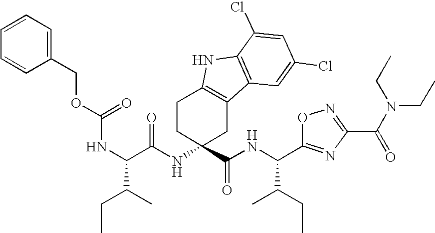
Compound 21 ((S)-1-{(R)-6,8-Dichloro-3-[(S)-1-(3-cyano-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester 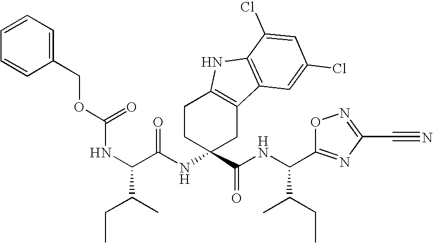
Compound 22 ((S)-1-{(S)-6,8-Dichloro-3-[(S)-1-(3-cyano-[1,2,4]oxadiazol-5-yl)-2-methyl-butylcarbamoyl]-2,3,4,9-tetrahydro-1H-carbazol-3-ylcarbamoyl}-2-methyl-butyl)-carbamic acid benzyl ester 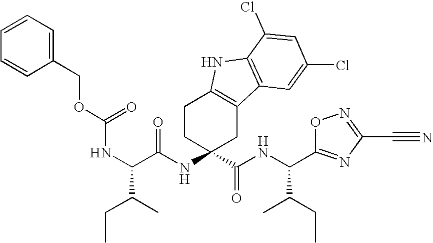
Compound 23 ((S)-1-{(R)-6,8-Dichloro-3-[(S)-2-methyl-1-(5-methyl-[1,3,4]oxadiazol-2-yl)-butylcarbamoyl]-2,3,4,9-tetrahydro-1H-carbazol-3-ylcarbamoyl}-2-methyl-butyl)-carbamic acid benzylester 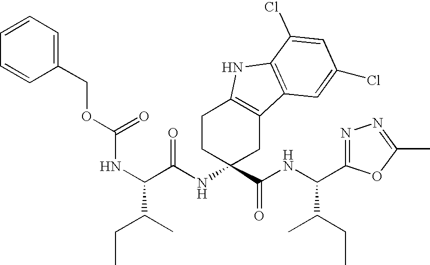
Compound 24 ((S)-1-{(S)-6,8-Dichloro-3-[(S)-2-methyl-1-(5-methyl-[1,3,4]oxadiazol-2-yl)-butylcarbamoyl]-2,3,4,9-tetrahydro-1H-carbazol-3-ylcarbamoyl}-2-methyl-butyl)-carbamic acid benzyl ester 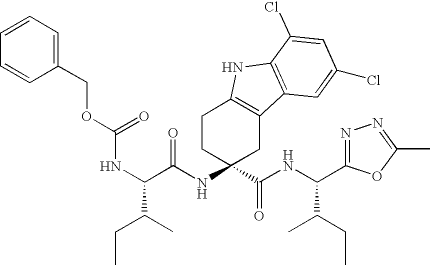
Compound 25 5-((S)-1-{[(R)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid methyl ester 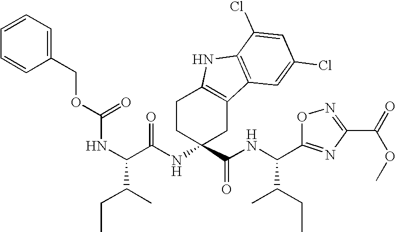
Compound 26 5-((S)-1-{[(S)-3-((S)-2-Benzyloxycarbonylamino-3-methyl-pentanoylamino)-6,8-dichloro-2,3,4,9-tetrahydro-1H-carbazole-3-carbonyl]-amino}-2-methyl-butyl)-[1,2,4]oxadiazole-3-carboxylic acid methyl ester 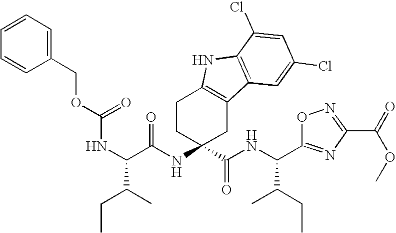
Compound 27 [(S)-1-((R)-6,8-Dichloro-3-{(S)-1-[5-(3-ethyl-ureido)-[1,3,4]oxadiazol-2-yl]-2-methyl-butylcarbamoyl}-2,3,4,9-tetrahydro-1H-carbazol-3-ylcarbamoyl)-2-methyl-butyl]-carbamic acid benzyl ester 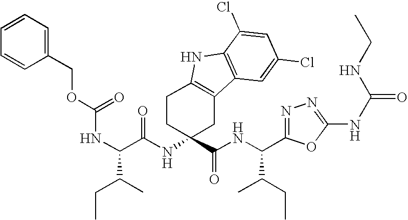
Compound 28 5-{(S)-1-[((R)-3-{(S)-2-[2-(2 Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester 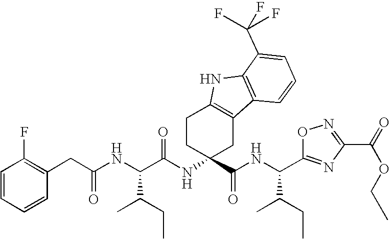
Compound 29 5-{(S)-1-[((S)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester 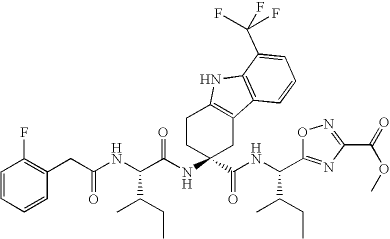
Compound 30 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide 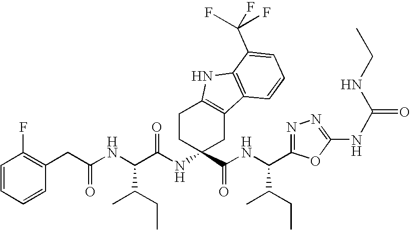
Compound 31 (S)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide 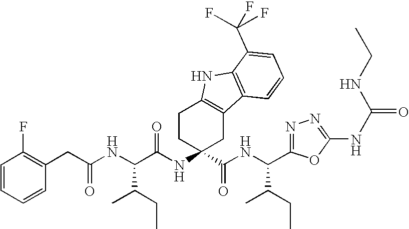
Compound 32 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(5-amino-[1,3,4]oxadiazol-2-yl)-2-methyl-butyl]-amide 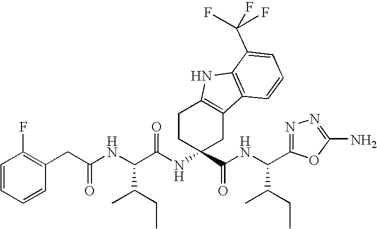
Compound 33 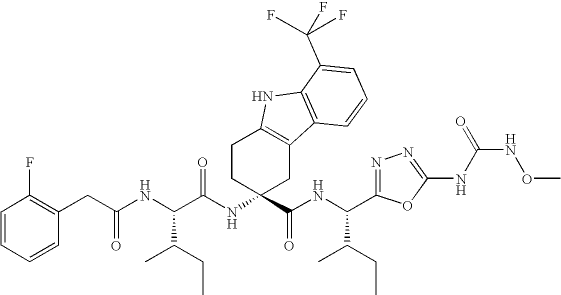
Compound 34 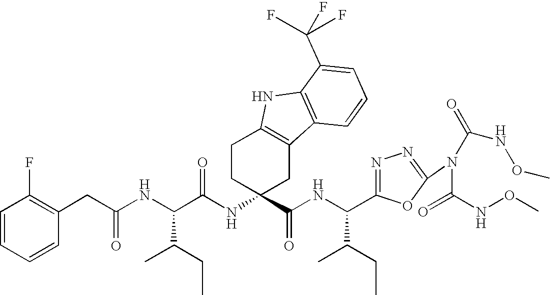
Compound 35 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrrolidin-1-ylmethyl)-carbamoyl]-butyl}-amide 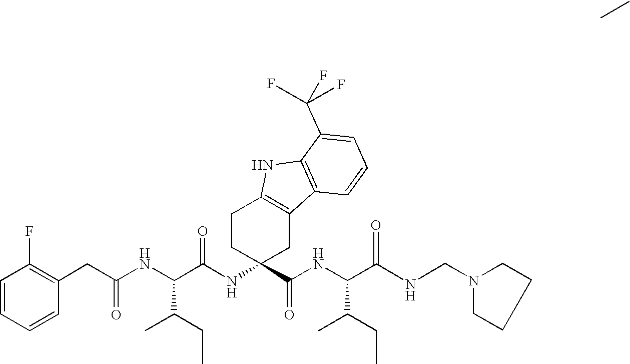
Compound 36 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(morpholin-4-ylmethyl)-carbamoyl]-butyl}-amide 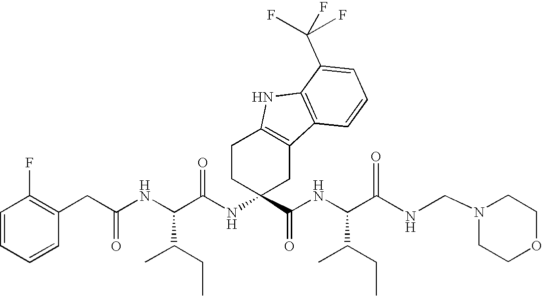
Compound 37 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(morpholin-4-ylmethyl)-thiocarbamoyl]-butyl}-amide 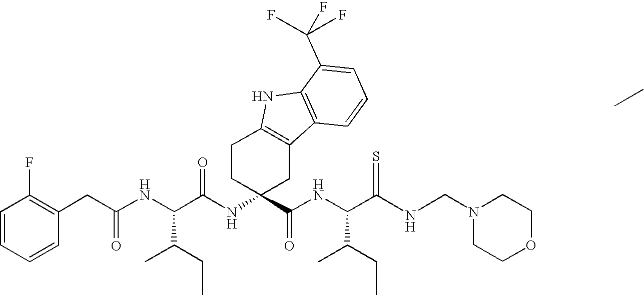
Compound 38 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-methoxycarbamoyl-2-methylbutyl)-amide 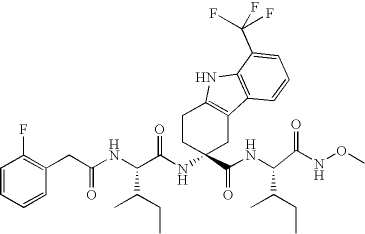
Compound 39 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(morpholine-4-carbonyl)-butyl]-amide 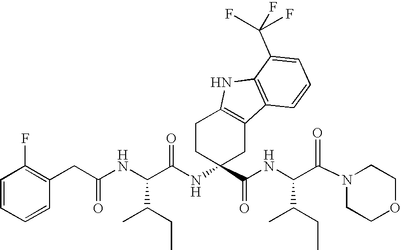
Compound 40 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-ethylcarbamoyl-2-methylbutyl)-amide 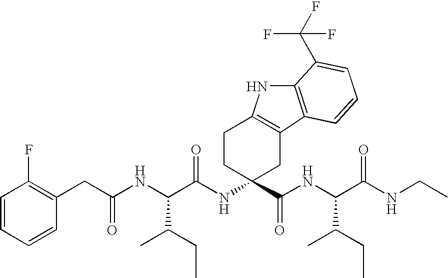
Compound 41 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-ethoxycarbamoyl-2-methylbutyl)-amide 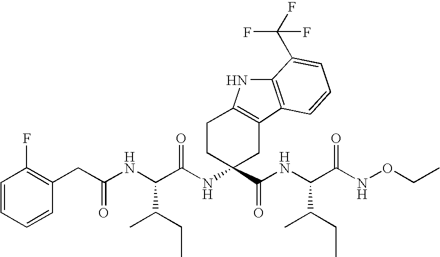
Compound 42 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylcarbamoyl)-butyl]-amide 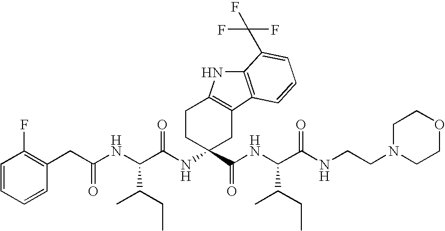
Compound 43 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-butylcarbamoyl)-2-methyl-butyl]-amide 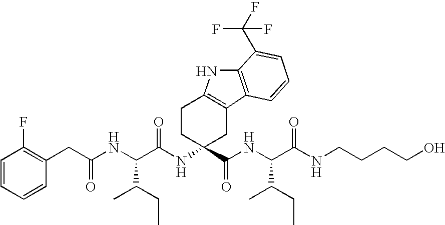
Compound 44 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-morpholin-4-yl-propylcarbamoyl)-butyl]-amide 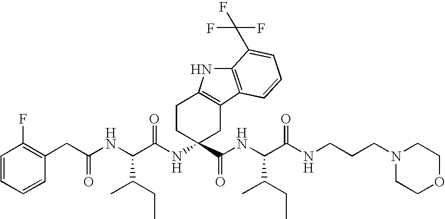
Compound 45 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(1-methyl-piperidin-4-yl-methyl)-carbamoyl]-butyl}-amide 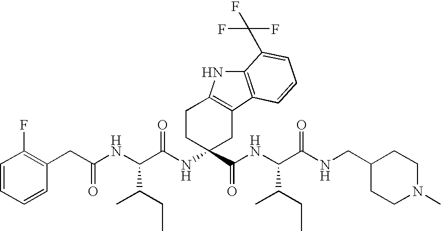
Compound 46 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(tetrahydro-pyran-4-ylmethyl)-carbamoyl]-butyl}-amide 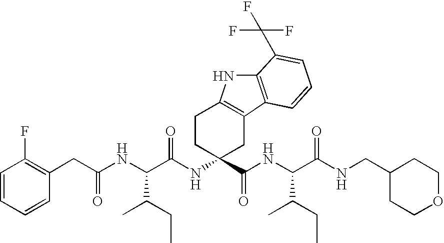
Compound 47 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylthiocarbamoyl)-butyl]-amide 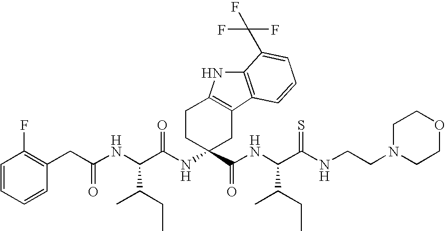
Compound 48 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(1-formyl-piperidin-4-ylmethyl)-carbamoyl]-2-methyl-butyl}-amide 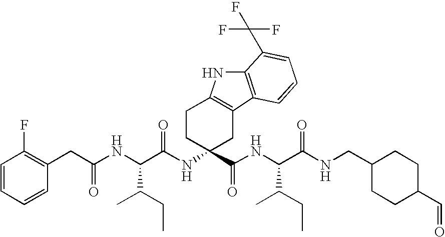
Compound 49 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(1-acetyl-piperidin-4-ylmethyl)-carbamoyl]-2-methyl-butyl}-amide 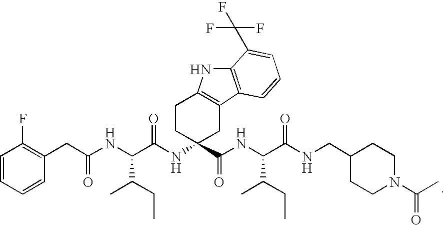
Compound 50 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-4-ylmethyl)carbamoyl]-butyl}-amide 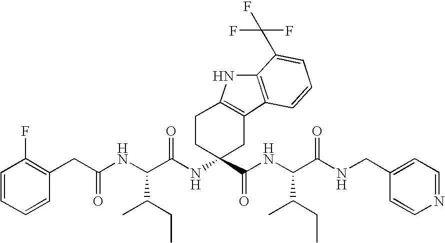
Compound 51 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-diethylamino-ethylcarbamoyl)-2-methyl-butyl]-amide 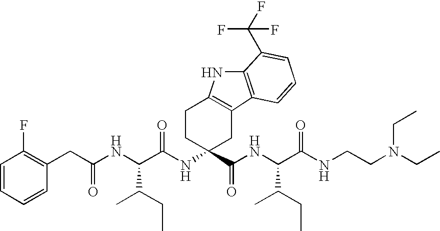
Compound 52 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(tetrahydro-pyran-4-ylmethyl)-thiocarbamoyl]-butyl}-amide 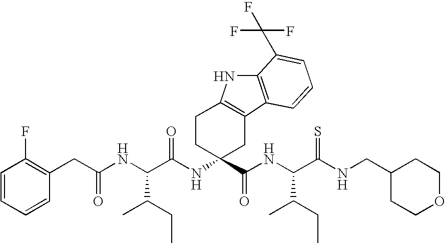
Compound 53 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-butylthiocarbamoyl)-2-methyl-butyl]-amide 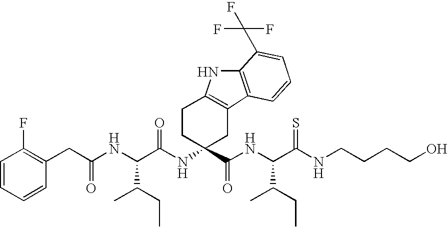
Compound 54 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-morpholin-4-yl-ethylthiocarbamoyl)-butyl]-amide 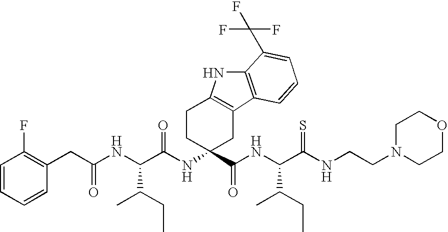
Compound 55 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid [(S)-2-methyl-1-(2-morpholin-4-yl-ethylcarbamoyl)-butyl]-amide 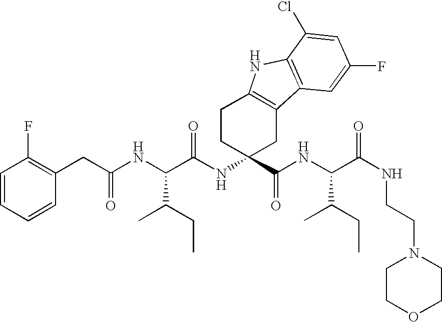
Compound 56 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(piperidin-4-ylmethyl)-carbamoyl]-butyl}-amide 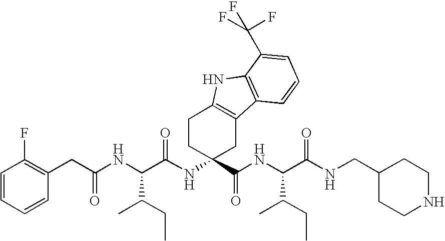
Compound 57 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-hydroxy-ethylcarbamoyl)-2-methyl-butyl]-amide 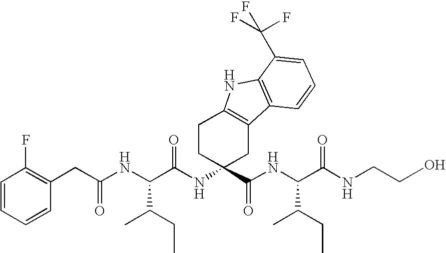
Compound 58 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide 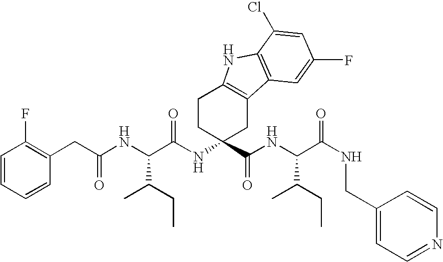
Compound 59 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(5-hydroxy-pentylcarbamoyl)-2-methyl-butyl]-amide 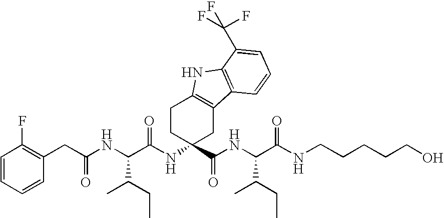
Compound 60 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-4-ylmethyl)thiocarbamoyl]-butyl}-amide 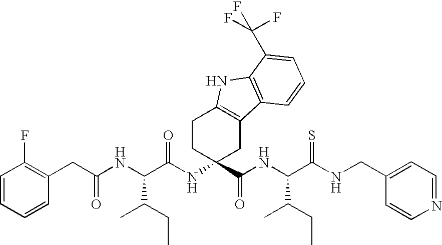
Compound 61 (R)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid [(S)-2-methyl-1-(2-morpholin-4-yl-ethylthiocarbamoyl)-butyl]-amide 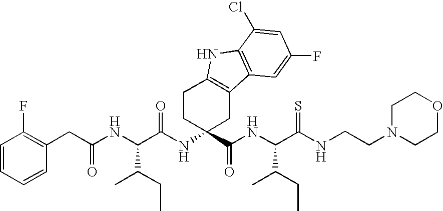
Compound 62 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-methyl-1-{[(tetrahydro-pyran-4-ylmethyl)-amino]-methyl}-butyl)-amide 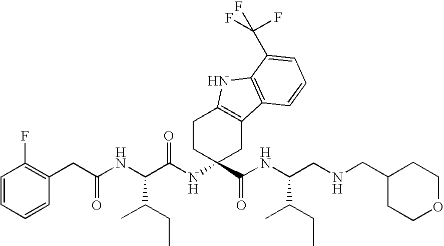
Compound 63 (4-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-butyl)phosphonic acid diethyl ester 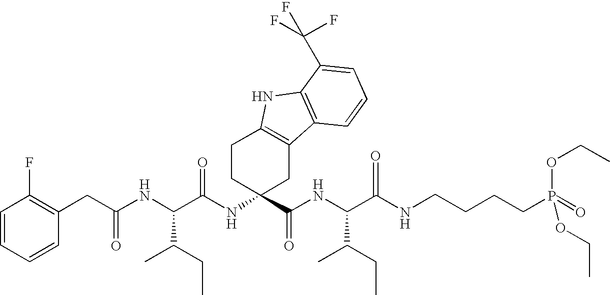
Compound 64 (4-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-butyl)phosphonic acid 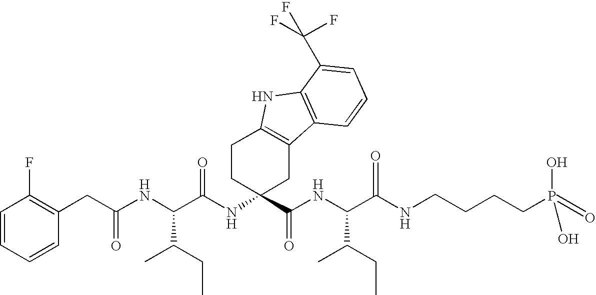
Compound 66 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(2-morpholin-4-yl-ethylamino)-methyl]-butyl}-amide 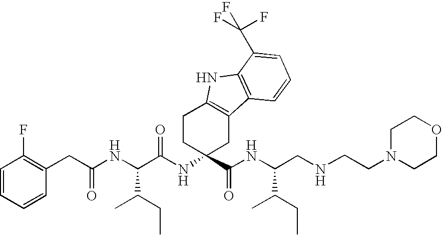
Compound 67 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-phenylcarbamoyl)-2-methyl-butyl]-amide 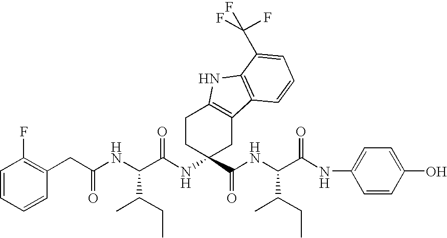
Compound 68 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-methoxy-phenylcarbamoyl)-2-methyl-butyl]-amide 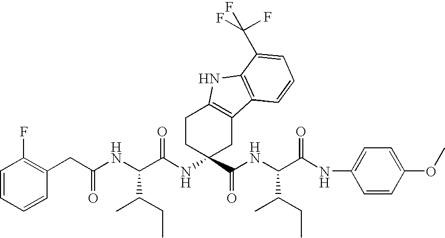
Compound 69 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-phenylcarbamoyl)-2-methyl-butyl]-amide 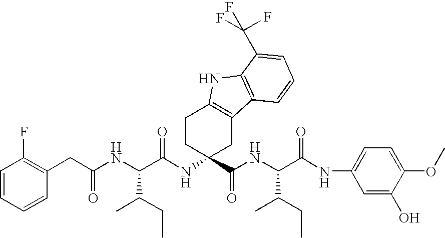
Compound 70 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2,4-dihydroxy-phenylcarbamoyl)-2-methyl-butyl]-amide 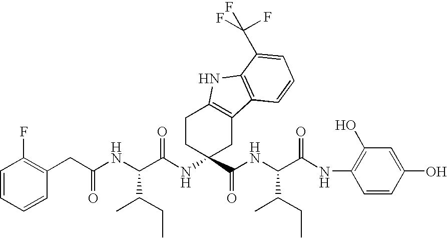
Compound 71 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-hydroxy-4-methoxy-phenylcarbamoyl)-2-methyl-butyl]-amide 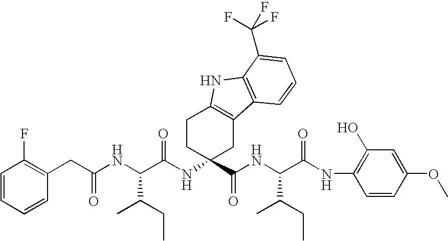
Compound 72 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2,4,6-trimethoxy-phenylcarbamoyl)-butyl]-amide 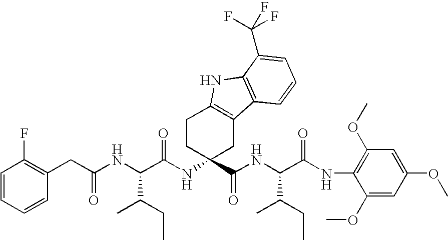
Compound 73 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-cyclohexylcarbamoyl)-2-methyl-butyl]-amide 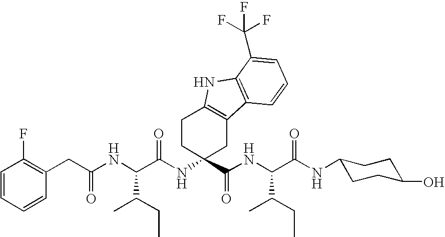
Compound 74 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-imidazol-1-yl-propylthiocarbamoyl)-2-methyl-butyl]-amide 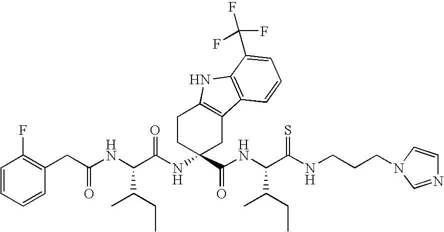
Compound 75 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(R)-1-(3-imidazol-1-yl-propylthiocarbamoyl)-2-methyl-butyl]-amide 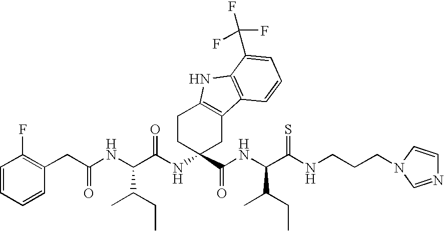
Compound 76 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-thiophen-2-yl-ethylthiocarbamoyl)-butyl]-amide 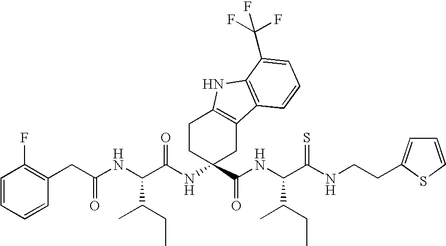
Compound 77 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyridin-3-ylmethyl)thiocarbamoyl]-butyl}-amide 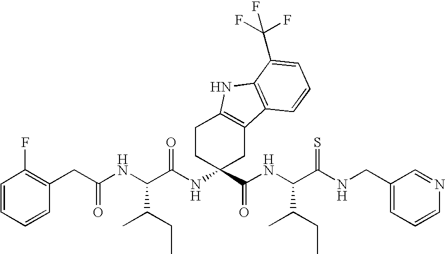
Compound 78 ((S)-1-{(R)-6,8-Dichloro-3-[(S)-3-methyl-1-(1H-tetrazol-5-yl)butylcarbamoyl]-2,3,4,9-tetrahydro-1H-carbazol-3-ylcarbamoyl}-2-methyl-butyl)-carbamic acid benzyl ester 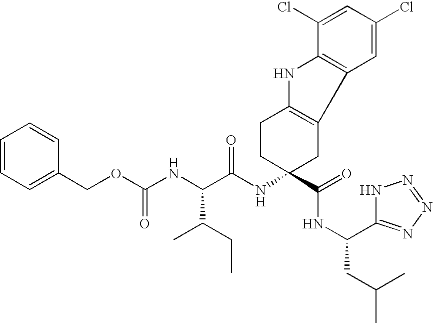
Compound 80 5-{(S)-1-[(3-{2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester 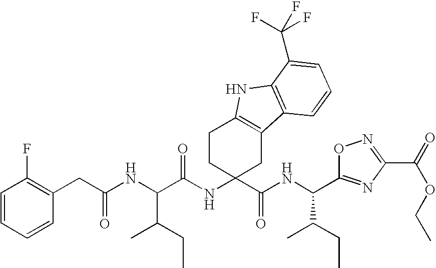
Compound 81 5-{(S)-1-[((R)-3-{(R)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-2-methyl-butyl}-[1,2,4]oxadiazole-3-carboxylic acid ethyl ester 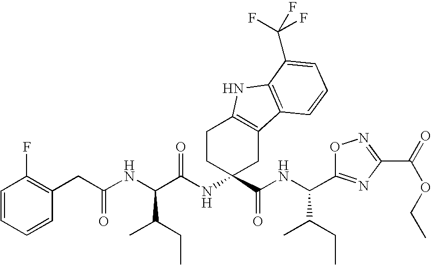
Compound 82 (R)-3-{(R)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide 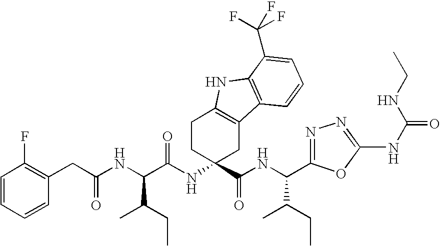
Compound 83 (S)-3-{(R)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[5-(3-ethyl-ureido)-[1,3,4]oxadiazol-2-yl]-2-methyl-butyl}-amide 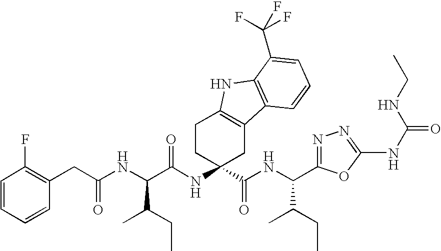
Compound 85 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(2-diethylamino-ethylcarbamoyl)methyl]-amide 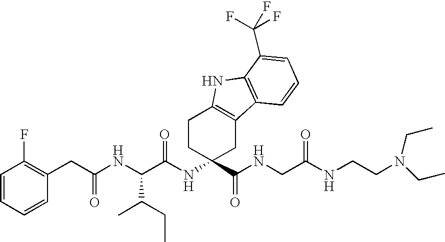
Compound 86 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(2-morpholin-4-yl-ethylcarbamoyl)methyl]-amide 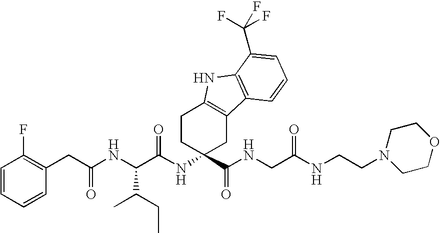
Compound 87 (S)-8-Chloro-6-fluoro-3-{(S)-2-[2-(2-fluoro-phenyl)-acetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide 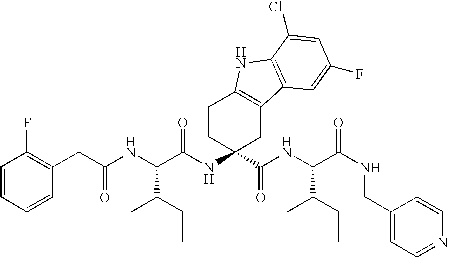
Compound 88 (R)-8-Chloro-6-fluoro-3-{(S)-2-[2-(2-fluoro-phenyl)thioacetylamino]-3-methyl-pentanoylamino}-2,3,4,9-tetrahydro-1H-carbazole-3-carboxylic acid {(S)-2-methyl-1-[(pyridin-4-ylmethyl)-carbamoyl]-butyl}-amide 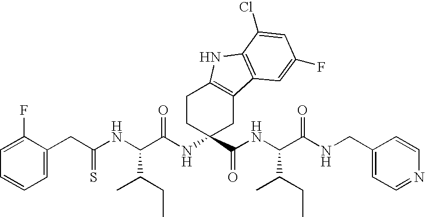
Compound 89 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyridin-4-yl-ethylthiocarbamoyl)-butyl]-amide 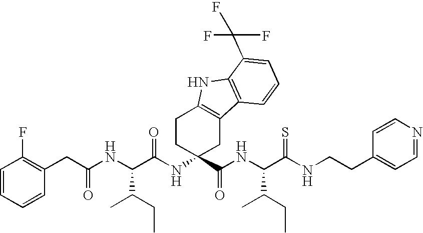
Compound 90 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-cyclohexylthiocarbamoyl)-2-methyl-butyl]-amide 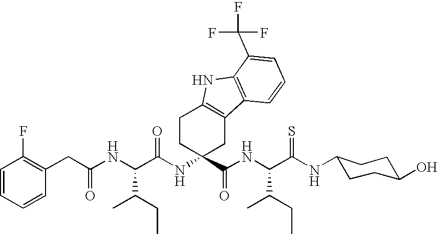
Compound 91 2-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-5-methoxy-benzoic acid 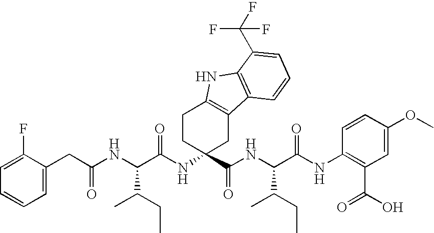
Compound 92 Phosphoric acid diethyl ester 5-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-2-methoxy-phenyl ester 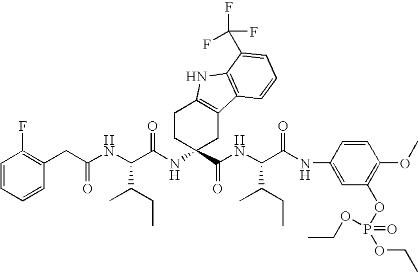
Compound 93 Dimethylamino-acetic acid 4-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-butyl ester 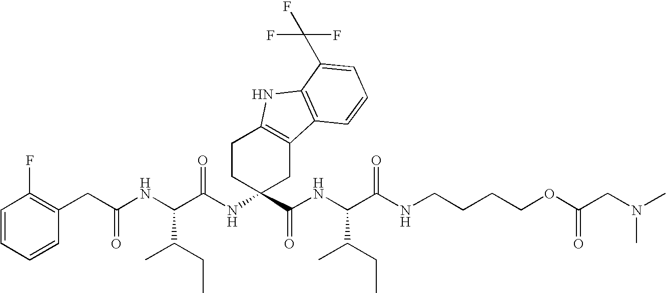
Compound 94 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-benzylcarbamoyl)-2-methyl-butyl]-amide 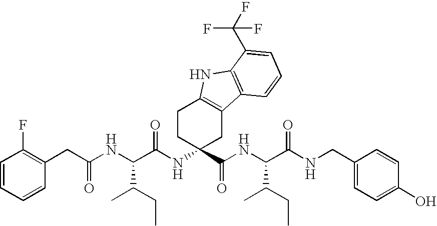
Compound 95 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-benzylcarbamoyl)-2-methyl-butyl]-amide 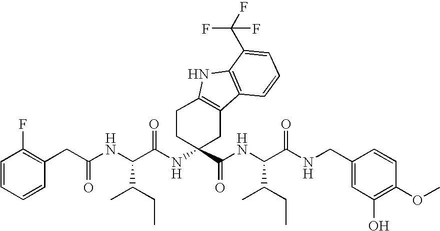
Compound 96 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-hydroxy-3-methoxy-benzylcarbamoyl)-2-methyl-butyl]-amide 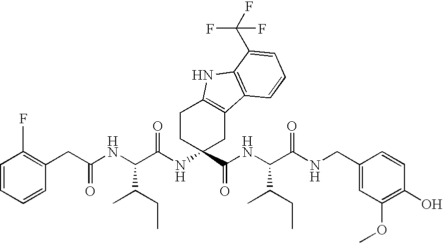
Compound 97 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(4-methoxy-phenylthiocarbamoyl)-2-methyl-butyl]-amide 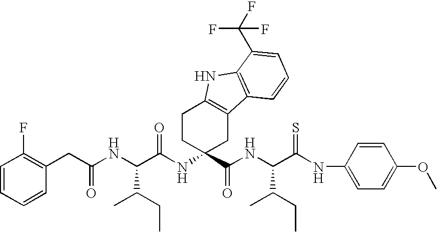
Compound 98 5-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-2-methoxy-benzoic acid 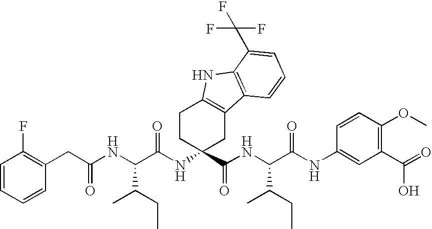
Compound 99 5-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanethioylamino}-2-methoxy-benzoic acid 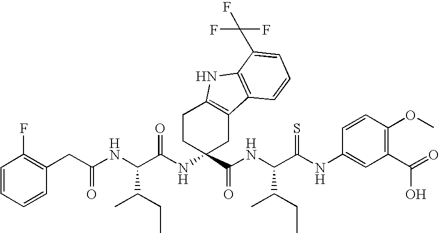
Compound 100 Carbonic acid 4-{(S)-2-[((R)-3-{(S)-2-[2-(2-fluoro-phenyl)acetylamino]-3-methyl-pentanoylamino}-8-trifluoromethyl-2,3,4,9-tetrahydro-1H-carbazole-3-carbonyl)-amino]-3-methyl-pentanoylamino}-butyl ester 2-[2-(2-methoxy-ethoxy)-ethoxy]-ethyl ester 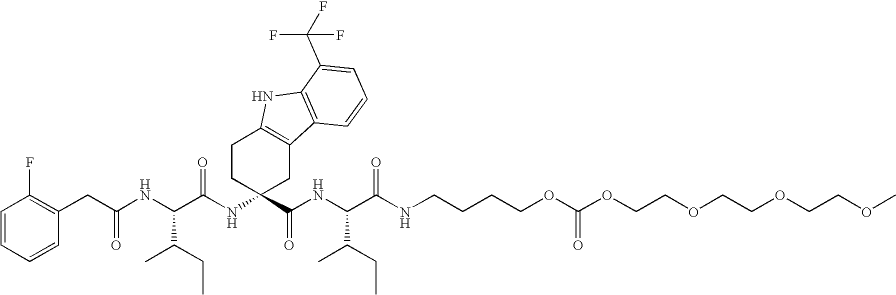
Compound 101 Phosphoric acid mono-(4-{(S)-2-[((R)-3-{(S)-2-[2-(2-fluorophenyl)-acetylamino]-3-methyl-pentanoylamino}-8-trifluoromethyl-2,3,4,9-tetrahydro-1H-carbazole-3-carbonyl)amino]-3-methyl-pentanoylamino}-butyl) ester 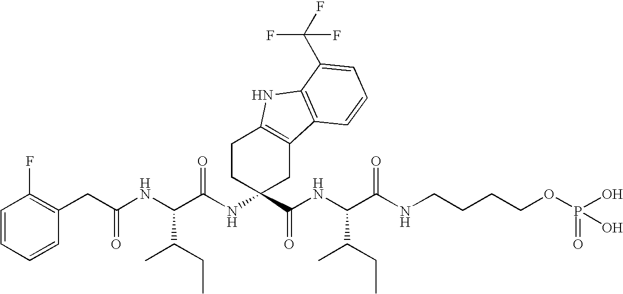
Compound 102 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-propylcarbamoyl)-2-methyl-butyl]-amide 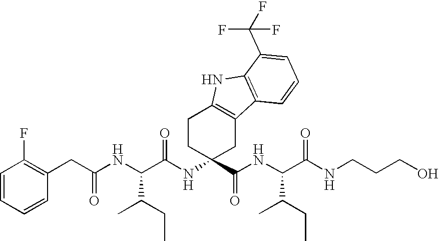
Compound 103 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-benzylthiocarbamoyl-2-methyl-butyl)-amide 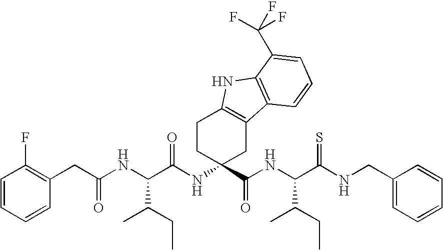
Compound 104 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(2-methoxy-pyridin-4-ylmethyl)-thiocarbamoyl]-2-methyl-butyl}-amide 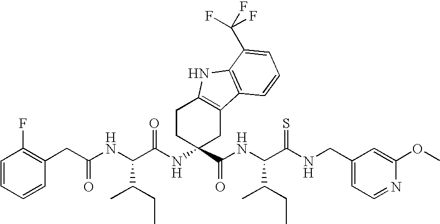
Compound 105 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrimidin-5-ylmethyl)-thiocarbamoyl]-butyl}-amide 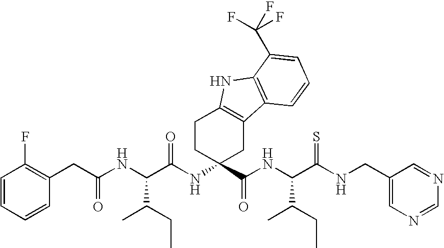
Compound 106 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrazin-2-ylmethyl)-thiocarbamoyl]-butyl}-amide 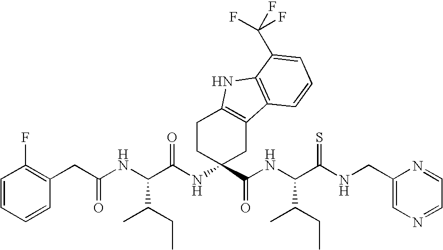
Compound 107 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(6-chloro-pyridin-3-ylmethyl)thiocarbamoyl]-2-methyl-butyl}-amide 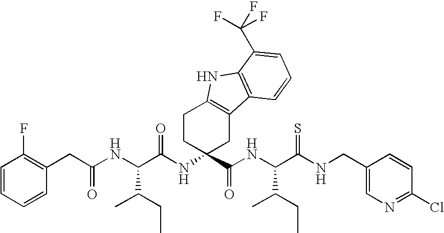
Compound 108 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyridin-3-yl-ethylthiocarbamoyl)-butyl]-amide 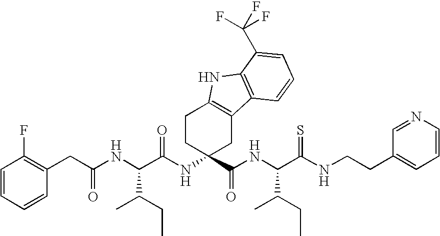
Compound 109 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(pyrimidin-4-ylmethyl)-thiocarbamoyl]-butyl}-amide 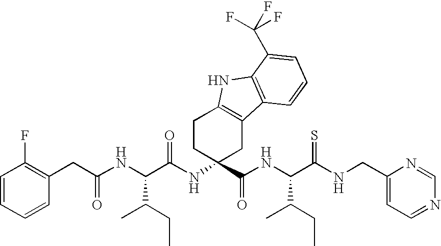
Compound 110 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-imidazol-1-yl-ethylthiocarbamoyl)-2-methyl-butyl]-amide 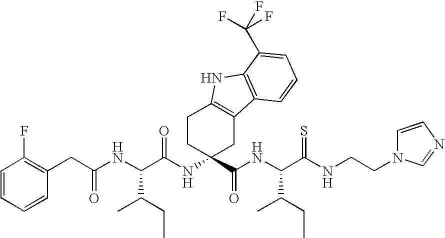
Compound 111 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-pyrazol-1-yl-ethylthiocarbamoyl)-butyl]-amide 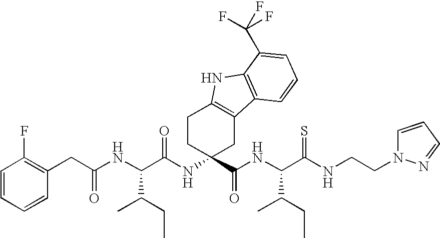
Compound 112 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-[1,2,4]triazol-1-yl-ethylthiocarbamoyl)-butyl]-amide 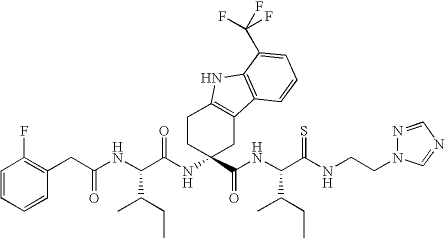
Compound 113 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-[1,2,4]triazol-1-yl-propylthiocarbamoyl)-butyl]-amide 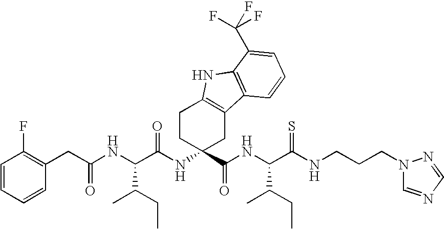
Compound 114 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(4-[1,2,4]triazol-1-yl-butylthiocarbamoyl)-butyl]-amide 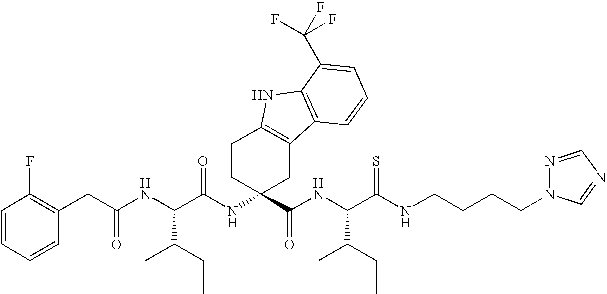
Compound 115 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(furan-2-ylmethyl)thiocarbamoyl]-2-methyl-butyl}-amide 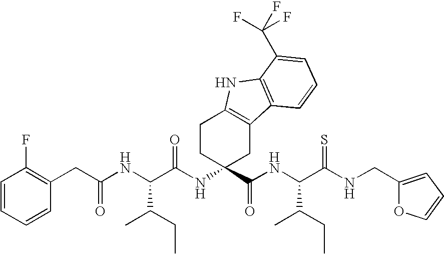
Compound 116 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(furan-3-ylmethyl)thiocarbamoyl]-2-methyl-butyl}-amide 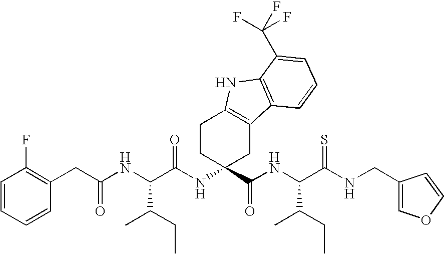
Compound 117 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(2-furan-2-yl-ethylthiocarbamoyl)-2-methyl-butyl]-amide 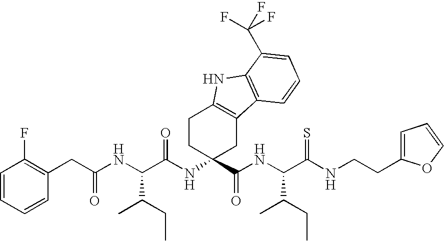
Compound 118 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(5-methyl-[1,3,4]oxadiazol-2-ylmethyl)-thiocarbamoyl]-butyl}-amide 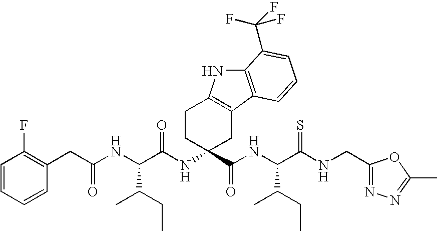
Compound 119 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[2-(tetrahydro-pyran-4-yl)-ethylthiocarbamoyl]-butyl}-amide 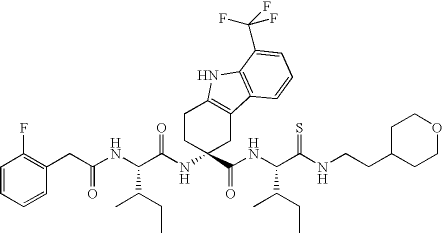
Compound 120 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(tetrahydro-pyran-4-yl-thiocarbamoyl)-butyl]-amide 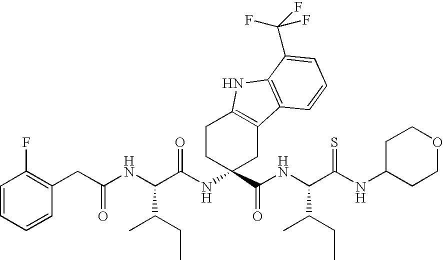
Compound 121 5-{(S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoylamino}-2-hydroxy-benzoic acid 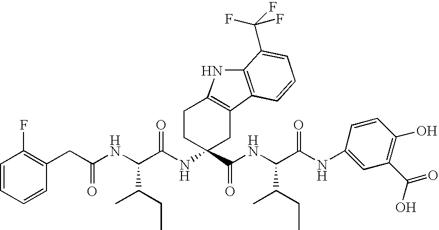
Compound 122 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-fluoro-4-hydroxy-phenylcarbamoyl)-2-methyl-butyl]-amide 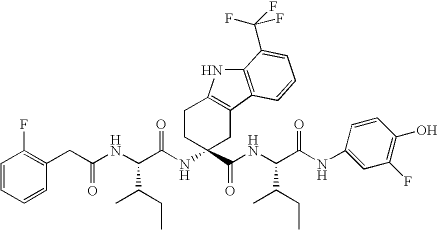
Compound 123 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-benzylthiocarbamoyl)-2-methyl-butyl]-amide 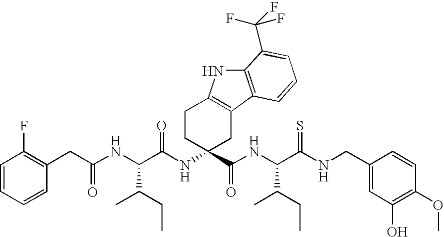
Compound 124 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-1-hydrazinocarbonyl-2-methylbutyl)-amide 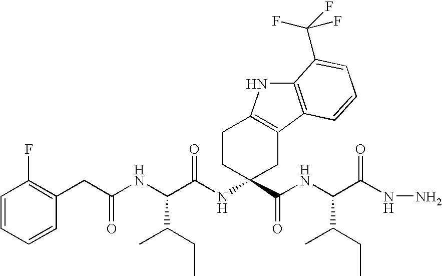
Compound 125 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-hydroxy-4-methoxy-phenylthiocarbamoyl)-2-methyl-butyl]-amide 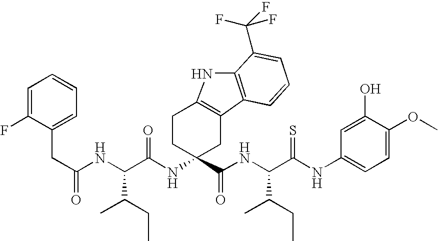
Compound 126 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-oxo-pyrrolidin-3-yl)-amide 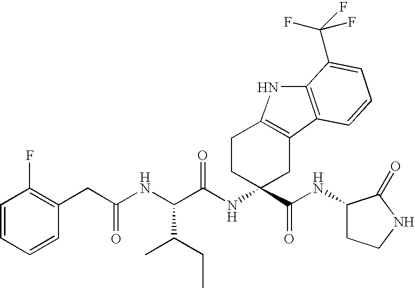
Compound 127 (S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-imidazol-1-yl-propyl ester 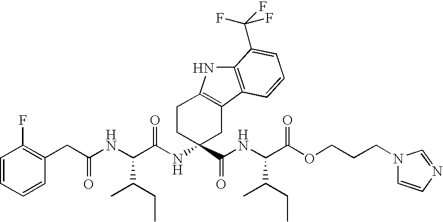
Compound 128 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-imidazol-1-yl-propyl ester 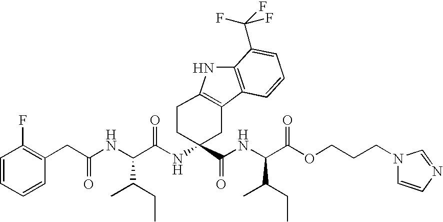
Compound 129 ((S)-1-{(R)-6,8-Dichloro-3-[(S)-1-(4-hydroxy-benzylcarbamoyl]-2-methyl-butylcarbamoyl]-2,3,4,9-tetrahydro-1H-carbazol-3-ylcarbamoyl}-2-methyl-butyl)-carbamic acid benzyl ester 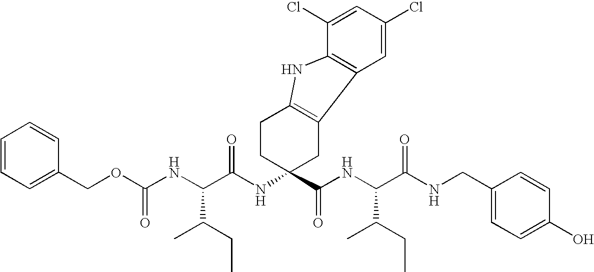
Compound 130 ((S)-1-{(S)-6,8-Dichloro-3-[(S)-1-(4-hydroxy-benzylcarbamoyl)-2-methyl-butylcarbamoyl]-2,3,4,9-tetrahydro-1H-carbazol-3-ylcarbamoyl}-2-methyl-butyl)-carbamic acid benzyl ester 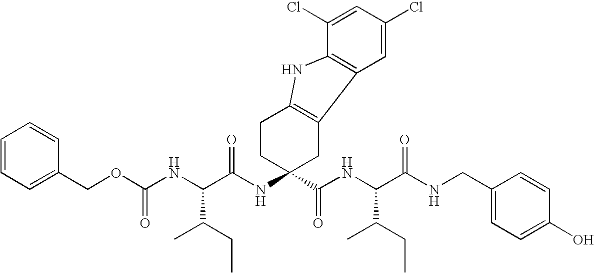
Compound 131 (S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid pyridin-4-ylmethyl ester 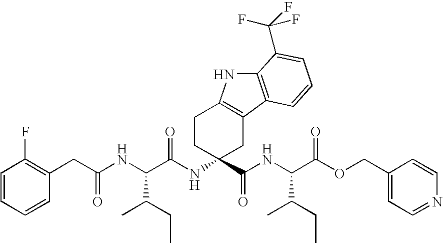
Compound 132 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid pyridin-4-ylmethyl ester 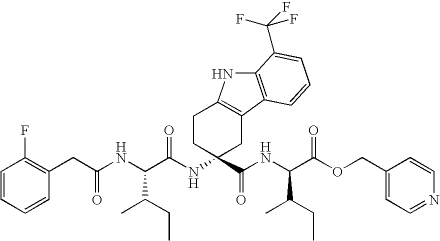
Compound 133 2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 2-dimethylamino-ethyl ester 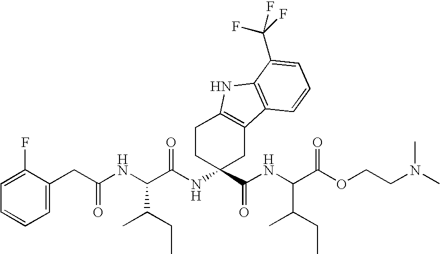
Compound 134 (S)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-hydroxy-4-methoxy-benzyl ester 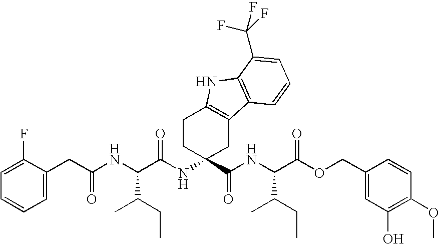
Compound 135 (R)-2-[((R)-3-{(S)-2-[2-(2-Fluoro-phenyl)-acetylamino]-3-methyl-pentanoylamino}-8-trifluoromethyl-2,3,4,9-tetrahydro-1H-carbazole-3-carbonyl)-amino]-3-methyl-pentanoic acid 3-hydroxy-4-methoxy-benzyl ester 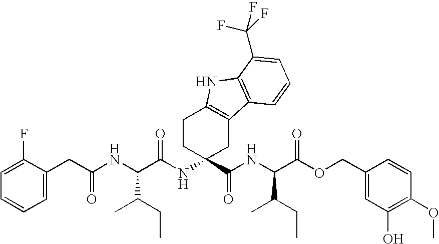
Compound 136 [(S)-1-((S)-6,8-Dichloro-3-{(S)-2-methyl-1-[(tetrahydro-pyran-4-ylmethyl)-carbamoyl]-butylcarbamoyl}-2,3,4,9-tetrahydro-1H-carbazol-3-ylcarbamoyl)-2-methyl-butyl]-carbamic acid benzyl ester 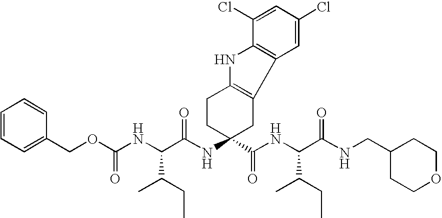
Compound 137 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(quinolin-6-ylcarbamoyl)-butyl]-amide 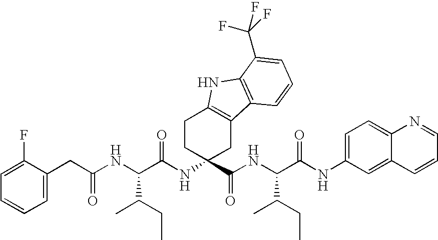
Compound 138 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(R)-2-methyl-1-(quinolin-6-ylcarbamoyl)-butyl]-amide 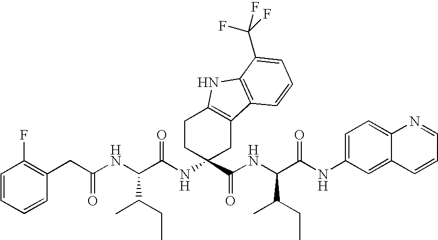
Compound 139 [(S)-1-((R)-6,8-Dichloro-3-{(S)-2-methyl-1-[(tetrahydro-pyran-4-ylmethyl)-thiocarbamoyl]-butylcarbamoyl}-2,3,4,9-tetrahydro-1H-carbazol-3-ylcarbamoyl)-2-methyl-butyl]-carbamic acid benzyl ester 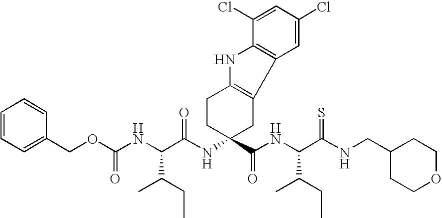
Compound 140 ((S)-1-{(R)-6,8-Dichloro-3-[(S)-1-(4-hydroxy-3-methoxy-phenylcarbamoyl)-2-methyl-butylcarbamoyl]-2,3,4,9-tetrahydro-1H-carbazol-3-ylcarbamoyl}-2-methyl-butyl)-carbamic acid benzyl ester 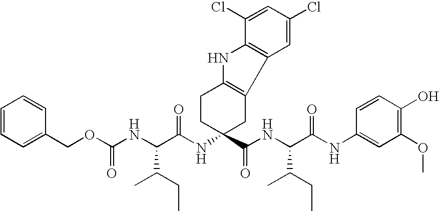
Compound 141 ((S)-1-{(S)-6,8-Dichloro-3-[(S)-1-(4-hydroxy-3-methoxy-phenylcarbamoyl)-2-methyl-butylcarbamoyl]-2,3,4,9-tetrahydro-1H-carbazol-3-ylcarbamoyl}-2-methyl-butyl)-carbamic acid benzyl ester 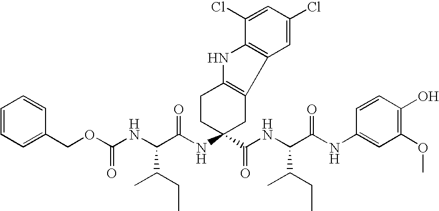
Compound 142 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-oxo-piperidin-3-yl)-amide 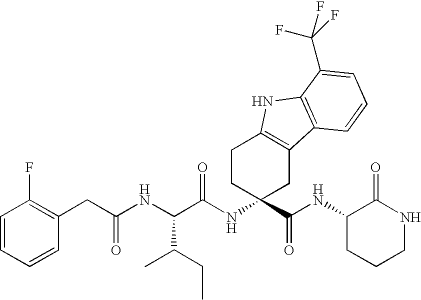
Compound 143 [(S)-1-((R)-6,8-Dichloro-3-{(S)-2-methyl-1-[(tetrahydro-pyran-4-ylmethyl)-carbamoyl]-butylcarbamoyl}-2,3,4,9-tetrahydro-1H-carbazol-3-ylcarbamoyl)-2-methyl-butyl]-carbamic acid benzyl ester 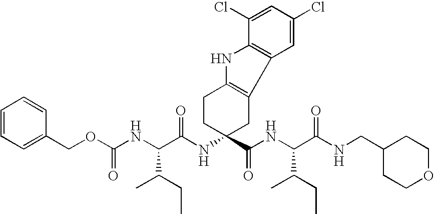
Compound 144 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(N′-phenyl-hydrazinocarbonyl)-butyl]-amide 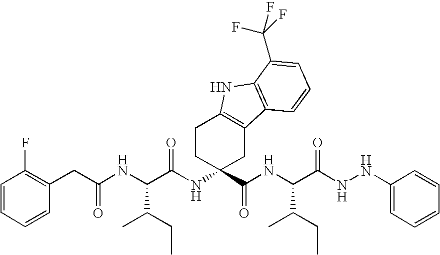
Compound 145 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-3-methylsulfanyl-1-thiocarbamoyl-propyl)-amide 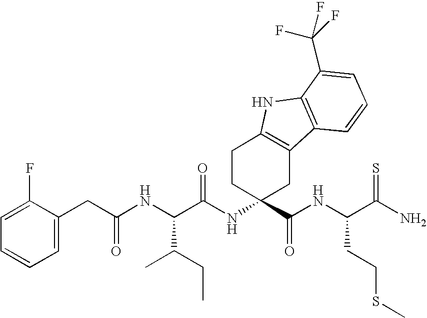
Compound 146 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(quinolin-5-ylcarbamoyl)-butyl]-amide 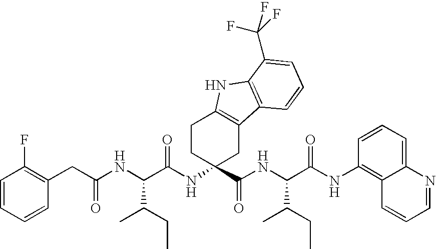
Compound 147 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(isoquinolin-5-ylcarbamoyl)-2-methyl-butyl]-amide 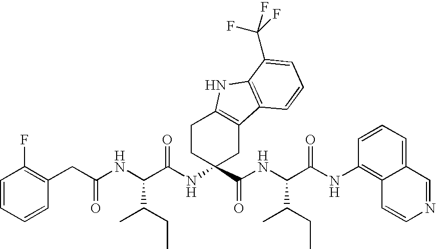
Compound 148 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(2-tetrahydro-pyran-4-yl-acetylamino)-methyl]-butyl}-amide 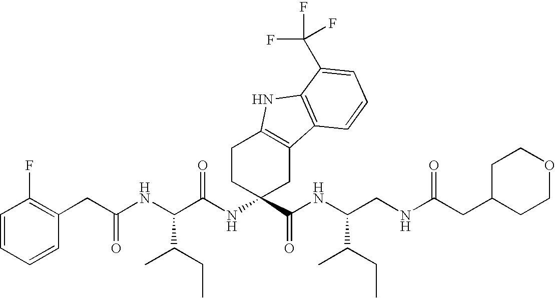
Compound 149 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((S)-2-methyl-1-{[(tetrahydro-pyran-4-carbonyl)-amino]-methyl}-butyl)-amide 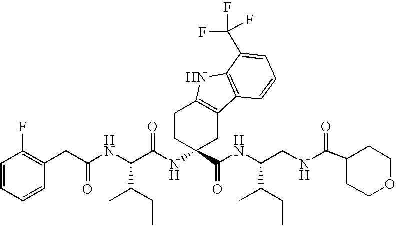
Compound 150 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(3-morpholin-4-yl-propionylamino)-methyl]-butyl}-amide 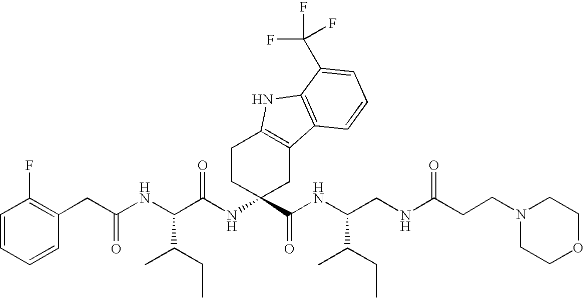
Compound 151 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[(3-imidazol-1-yl-propionylamino)-methyl]-2-methyl-butyl}-amide 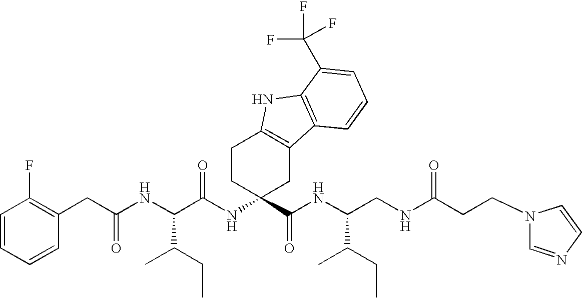
Compound 152 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[3-(tetrahydro-pyran-4-ylmethyl)-ureidomethyl]-butyl}-amide 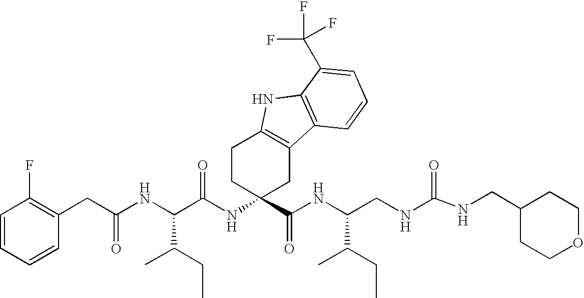
Compound 153 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(2-tetrahydro-pyran-4-yl-acetylamino)-butyl]-amide 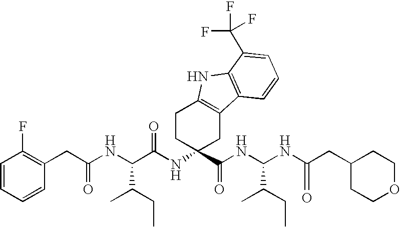
Compound 154 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-2-methyl-1-[(tetrahydro-pyran-4-carbonyl)-amino]-butyl}-amide 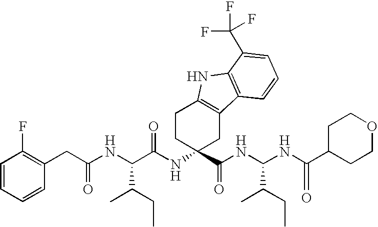
Compound 155 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-2-methyl-1-(3-morpholin-4-yl-propionylamino)-butyl]-amide 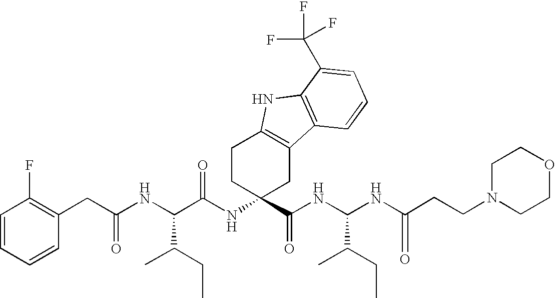
Compound 156 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(3-imidazol-1-yl-propionylamino)-2-methyl-butyl]-amide 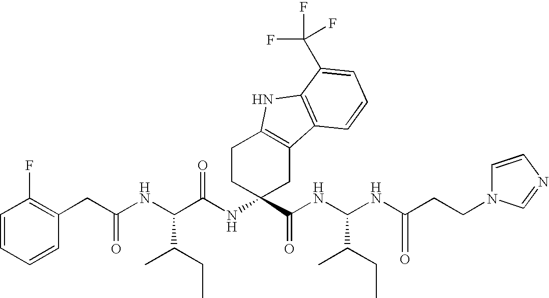
Compound 157 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(R)-2-methyl-1-[3-(tetrahydro-pyran-4-ylmethyl)-ureido]-butyl}-amide 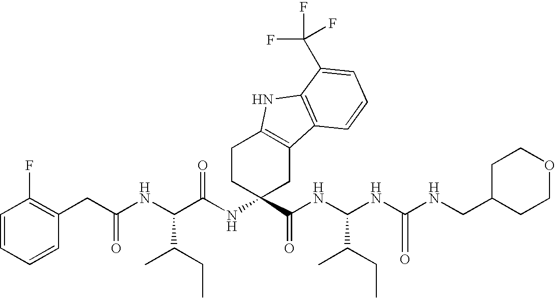
Compound 158 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid ((R)-2-methyl-1-{[(tetrahydro-pyran-4-ylmethyl)-carbamoyl]-methyl}-butyl)-amide 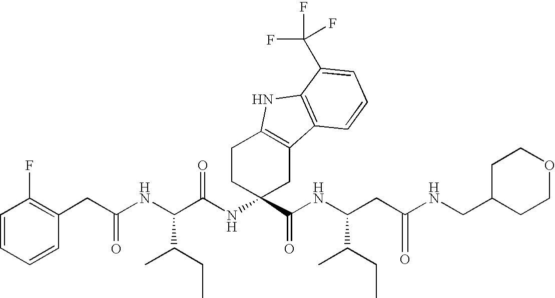
Compound 159 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-phenyl)hydrazinocarbonyl]-2-methyl-butyl}-amide 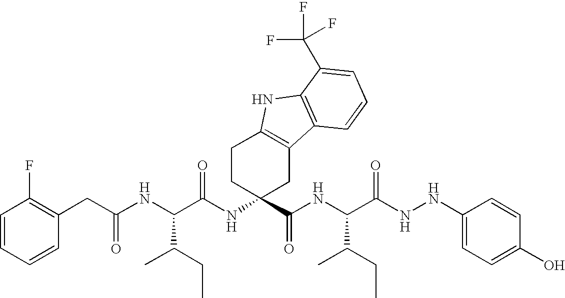
Compound 160 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-methoxy-phenyl)hydrazinocarbonyl]-2-methyl-butyl}-amide 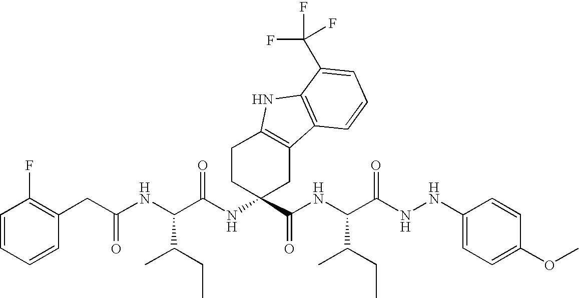
Compound 161 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(3-hydroxy-4-methoxybenzyl)-hydrazinocarbonyl]-2-methyl-butyl}-amide 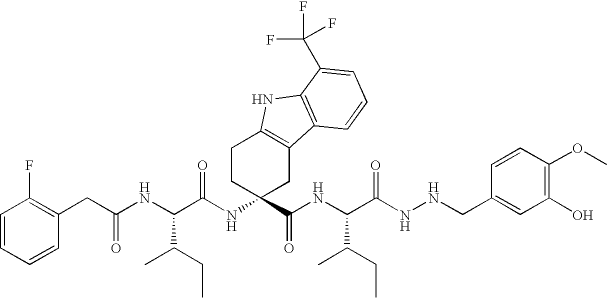
Compound 162 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-3-methoxybenzyl)-hydrazinocarbonyl]-2-methyl-butyl}-amide 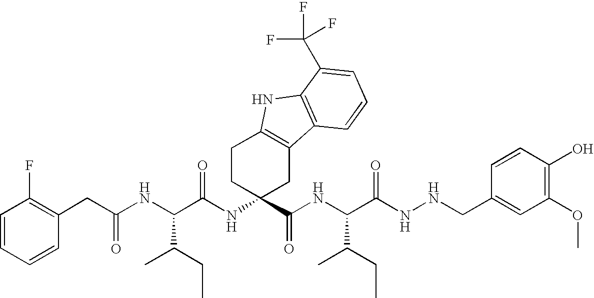
Compound 163 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid [(S)-1-(N′-acetyl-hydrazinocarbonyl)-2-methyl-butyl]-amide 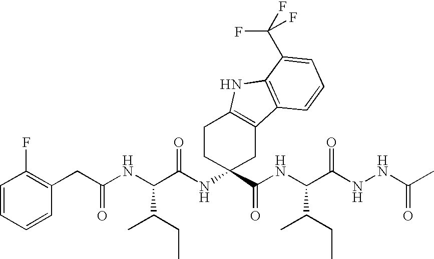
Compound 164 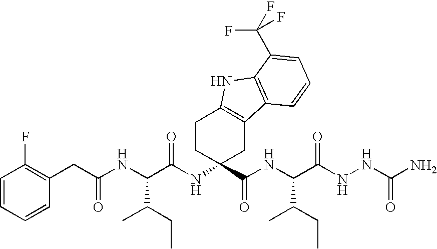
and
Compound 165 (R)-3-{(S)-2-[2-(2-Fluoro-phenyl)-acetylamino]-3-methyl-pentanoylamino}-8-trifluoromethyl-2,3,4,9-tetrahydro-1H-carbazole-3-carboxylic acid {(S)-1-[N′-(4-hydroxy-benzoyl)hydrazinocarbonyl]-2-methyl-butyl}-amide

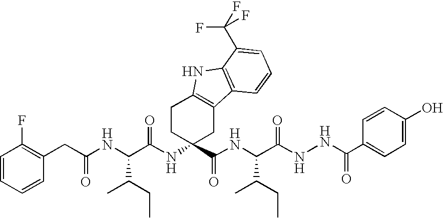

11. The method as claimed in claim 1, wherein the at least one LHRH antagonist is administered in a total monthly dose in an amount of 10 mg to 150 mg.

12. The method as claimed in claim 1, wherein the at least one LHRH antagonist is administered in a total monthly dose in an amount of 10 mg to 120 mg.

13. The method as claimed in claim 1, wherein the at least one LHRH antagonist is administered in a total monthly dose in in amount of 10 mg to 40 mg.

14. The method as claimed in claim 1, wherein the at least one LHRH antagonist is administered in a total monthly dose in an amount of 40 mg to 150 mg.

15. The method as claimed in claim 1, wherein the at least one LHRH antagonist is administered in a total monthly dose in an amount of 60 mg to 150 mg.

16. The method as claimed in claim 1, wherein the at least one LHRH antagonist is administered in a total monthly dose in an amount of 60 mg to 120 mg.

17. The method as claimed in claim 1, wherein the at least one LHRH antagonist is administered in a total monthly dose as one single monthly administration

18. The method as claimed in claim 1, wherein the at least one LHRH antagonist is administered twice a month or biweekly.

19. The method as claimed in claim 1, wherein the at least one LHRH antagonist is administered three-times a month.

20. The method as claimed in claim 1, wherein the at least one LHRH antagonist is administered four-times a month or weekly.

21. The method as claimed in claim 1, wherein the at least one LHRH antagonist is administered over a treatment period of 1 or 2 months followed by a treatment-free period of 1, 2, 3, 4 or 5 months.

22. The method as claimed in claim 21, wherein the treatment-free period is 2 months or 5 months.

23. The method as claimed in claim 1, wherein the at least one LHRH antagonist is administered in a dose of:
5 mg LHRH antagonist four-times a month or three-times a month or twice a month, or
10 mg LHRH antagonist four-times a month or three-times a month or twice a month, or
15 mg LHRH antagonist four-times a month or three-times a month or twice a month, or
30 mg LHRH antagonist four-times a month or three-times a month or twice a month, or
60 mg LHRH antagonist as one single administration followed by 30 mg LHRH antagonist as one single administration two weeks later, or
60 mg LHRH antagonist twice a month,
over a treatment period of 1 or 2 months followed by a treatment-free period of 1, 2, 3, 4 or 5 months.

24. The method as claimed in claim 23, wherein the treatment cycle is repeated after the end of the treatment-free period of the preceding treatment cycle once, twice, three-times, four-times, five-times or continuously and wherein each respective succeeding treatment cycle can be identical or different to each respective preceding treatment cycle.

25. The method as claimed in claim 1, wherein the at least one LHRH antagonist is administered as a single or multiple daily dose of 0.1 mg to 250 mg.

26. The method as claimed in claim 25, which is followed by a treatment-free period.

27. The method as claimed in claim 1, wherein the chemical castration is a testosterone castration caused by a testosterone blood level of equal or below 1.2 ng/mL.

28. The method as claimed in claim 1, wherein the chemical castration is a testosterone castration caused by a testosterone blood level of equal or below 0.5 ng/mL.

29. The method as claimed in claim 1, which further comprises administering at least one additional pharmacologically active substance.

30. The method as claimed in 22, wherein the at least one additional pharmacologically active substance is selected from the group consisting of smooth muscle relaxants, bladder relaxants, bladder smooth muscle relaxants, anticholinergic agents, muscarinic acetylcholine receptor antagonists, tricyclic antidepressants, Calcium antagonist, Calcium agonist, Calcium channel blockers, Calcium channel activators, Potassium antagonists, Potassium agonists, Potassium channel blockers, Potassium channel activators, alpha-adrenergic receptor antagonists, alpha-blockers, alpha-adrenoreceptor antagonists, β3-adrenoreceptor agonists, vanilloids, vanilloid receptor antagonists, and botulinum toxins.
The method as claimed in claim 22, wherein the at least one additional pharmacologically active substance is selected from the group consisting of oxybutynin, flavoxate, propantheline, dicyclomine, tolterodine, darifenancin, solifenancin, trospium chloride, fesoterodine, imidafenacin, PSD-506, imipramine; terodiline, terazosin, phenoxybenzamine, prazosin, tamsulosin, ritobegron, YM-178, solabegron, resiniferatoxin, and botulinum toxin A.
